SEQUENCE LISTING (8 sequences)

SEQ ID NO: 1 - protein, 46 aa, synthetic construct
PQSMLDQQRELARKREQERRRREAMAATIDMNFQSDLLSIFEENLF

SEQ ID NO: 2 - protein, 170 aa, synthetic construct
DLKIKNMGSWASLVQKHPTTPSSTAKSSSDSFEQFRRAAREKEEREKALKAQAEHAEKEKERLRQERMRSREDEDALEQARRAHEEARRRQEQQQQQRQEQQQQQQQQAAAVAAAATPQAQSSQPQSMLDQQRELARKREQERRRREAMAATIDMNFQSDLLSIFEENLF

SEQ ID NO: 3 - protein, 1362 aa, synthetic construct
MSAESGPGTRLRNLPVMGDGLETSQMSTTQAQAQPQPANAASTNPPPPETSNPNKPKRQTNQLQYLLRVVLKTLWKHQFAWPFQQPVDAVKLNLPDYYKIIKTPMDMGTIKKRLENNYYWNAQECIQDFNTMFTNCYIYNKPGDDIVLMAEALEKLFLQKINELPTEETEIMIVQAKGRGRGRKETGTAKPGVSTVPNTTQASTPPQTQTPQPNPPPVQATPHPFPAVTPDLIVQTPVMTVVPPQPLQTPPPVPPQPQPPPAPAPQPVQSHPPIIAATPQPVKTKKGVKRKADTTTPTTIDPIHEPPSLPPEPKTTKLGQRRESSRPVKPPKKDVPDSQQHPAPEKSSKVSEQLKCCSGILKEMFAKKHAAYAWPFYKPVDVEALGLHDYCDIIKHPMDMSTIKSKLEAREYRDAQEFGADVRLMFSNCYKYNPPDHEVVAMARKLQDVFEMRFAKMPDEPEEPVVAVSSPAVPPPTKVVAPPSSSDSSSDSSSDSDSSTDDSEEERAQRLAELQEQLKAVHEQLAALSQPQQNKPKKKEKDKKEKKKEKHKRKEEVEENKKSKAKEPPPKKTKKNNSSNSNVSKKEPAPMKSKPPPTYESEEEDKCKPMSYEEKRQLSLDINKLPGEKLGRVVHIIQSREPSLKNSNPDEIEIDFETLKPSTLRELERYVTSCLRKKRKPQAEKVDVIAGSSKMKGFSSSESESSSESSSSDSEDSETEMAPKSKKKGHPGREQKKHHHHHHQQMQQAPAPVPQQPPPPPQQPPPPPPPQQQQQPPPPPPPPSMPQQAAPAMKSSPPPFIATQVPVLEPQLPGSVFDPIGHFTQPILHLPQPELPPHLPQPPEHSTPPHLNQHAVVSPPALHNALPQQPSRPSNRAAALPPKPARPPAVSPALTQTPLLPQPPMAQPPQVLLEDEEPPAPPLTSMQMQLYLQQLQKVQPPTPLLPSVKVQSQPPPPLPPPPHPSVQQQLQQQPPPPPPPQPQPPPQQQHQPPPRPVHLQPMQFSTHIQQPPPPQGQQPPHPPPGQQPPPPQPAKPQQVIQHHHSPRHHKSDPYSTGHLREAPSPLMIHSPQMSQFQSLTHQSPPQQNVQPKKQELRAASVVQPQPLVVVKEEKIHSPIIRSEPFSPSLRPEPPKHPESIKAPVHLPQRPEMKPVDVGRPVIRPPEQNAPPPGAPDKDKQKQEPKTPVAPKKDLKIKNMGSWASLVQKHPTTPSSTAKSSSDSFEQFRRAAREKEEREKALKAQAEHAEKEKERLRQERMRSREDEDALEQARRAHEEARRRQEQQQQQRQEQQQQQQQQAAAVAAAATPQAQSSQPQSMLDQQRELARKREQERRRREAMAATIDMNFQSDLLSIFEENLF

SEQ ID NO: 4 - protein, 1316 aa, synthetic construct
MSAESGPGTRLRNLPVMGDGLETSQMSTTQAQAQPQPANAASTNPPPPETSNPNKPKRQTNQLQYLLRVVLKTLWKHQFAWPFQQPVDAVKLNLPDYYKIIKTPMDMGTIKKRLENNYYWNAQECIQDFNTMFTNCYIYNKPGDDIVLMAEALEKLFLQKINELPTEETEIMIVQAKGRGRGRKETGTAKPGVSTVPNTTQASTPPQTQTPQPNPPPVQATPHPFPAVTPDLIVQTPVMTVVPPQPLQTPPPVPPQPQPPPAPAPQPVQSHPPIIAATPQPVKTKKGVKRKADTTTPTTIDPIHEPPSLPPEPKTTKLGQRRESSRPVKPPKKDVPDSQQHPAPEKSSKVSEQLKCCSGILKEMFAKKHAAYAWPFYKPVDVEALGLHDYCDIIKHPMDMSTIKSKLEAREYRDAQEFGADVRLMFSNCYKYNPPDHEVVAMARKLQDVFEMRFAKMPDEPEEPVVAVSSPAVPPPTKVVAPPSSSDSSSDSSSDSDSSTDDSEEERAQRLAELQEQLKAVHEQLAALSQPQQNKPKKKEKDKKEKKKEKHKRKEEVEENKKSKAKEPPPKKTKKNNSSNSNVSKKEPAPMKSKPPPTYESEEEDKCKPMSYEEKRQLSLDINKLPGEKLGRVVHIIQSREPSLKNSNPDEIEIDFETLKPSTLRELERYVTSCLRKKRKPQAEKVDVIAGSSKMKGFSSSESESSSESSSSDSEDSETEMAPKSKKKGHPGREQKKHHHHHHQQMQQAPAPVPQQPPPPPQQPPPPPPPQQQQQPPPPPPPPSMPQQAAPAMKSSPPPFIATQVPVLEPQLPGSVFDPIGHFTQPILHLPQPELPPHLPQPPEHSTPPHLNQHAVVSPPALHNALPQQPSRPSNRAAALPPKPARPPAVSPALTQTPLLPQPPMAQPPQVLLEDEEPPAPPLTSMQMQLYLQQLQKVQPPTPLLPSVKVQSQPPPPLPPPPHPSVQQQLQQQPPPPPPPQPQPPPQQQHQPPPRPVHLQPMQFSTHIQQPPPPQGQQPPHPPPGQQPPPPQPAKPQQVIQHHHSPRHHKSDPYSTGHLREAPSPLMIHSPQMSQFQSLTHQSPPQQNVQPKKQELRAASVVQPQPLVVVKEEKIHSPIIRSEPFSPSLRPEPPKHPESIKAPVHLPQRPEMKPVDVGRPVIRPPEQNAPPPGAPDKDKQKQEPKTPVAPKKDLKIKNMGSWASLVQKHPTTPSSTAKSSSDSFEQFRRAAREKEEREKALKAQAEHAEKEKERLRQERMRSREDEDALEQARRAHEEARRRQEQQQQQRQEQQQQQQQQAAAVAAAATPQAQSSQ

SEQ ID NO: 5 - protein, 1192 aa, synthetic construct
MSAESGPGTRLRNLPVMGDGLETSQMSTTQAQAQPQPANAASTNPPPPETSNPNKPKRQTNQLQYLLRVVLKTLWKHQFAWPFQQPVDAVKLNLPDYYKIIKTPMDMGTIKKRLENNYYWNAQECIQDFNTMFTNCYIYNKPGDDIVLMAEALEKLFLQKINELPTEETEIMIVQAKGRGRGRKETGTAKPGVSTVPNTTQASTPPQTQTPQPNPPPVQATPHPFPAVTPDLIVQTPVMTVVPPQPLQTPPPVPPQPQPPPAPAPQPVQSHPPIIAATPQPVKTKKGVKRKADTTTPTTIDPIHEPPSLPPEPKTTKLGQRRESSRPVKPPKKDVPDSQQHPAPEKSSKVSEQLKCCSGILKEMFAKKHAAYAWPFYKPVDVEALGLHDYCDIIKHPMDMSTIKSKLEAREYRDAQEFGADVRLMFSNCYKYNPPDHEVVAMARKLQDVFEMRFAKMPDEPEEPVVAVSSPAVPPPTKVVAPPSSSDSSSDSSSDSDSSTDDSEEERAQRLAELQEQLKAVHEQLAALSQPQQNKPKKKEKDKKEKKKEKHKRKEEVEENKKSKAKEPPPKKTKKNNSSNSNVSKKEPAPMKSKPPPTYESEEEDKCKPMSYEEKRQLSLDINKLPGEKLGRVVHIIQSREPSLKNSNPDEIEIDFETLKPSTLRELERYVTSCLRKKRKPQAEKVDVIAGSSKMKGFSSSESESSSESSSSDSEDSETEMAPKSKKKGHPGREQKKHHHHHHQQMQQAPAPVPQQPPPPPQQPPPPPPPQQQQQPPPPPPPPSMPQQAAPAMKSSPPPFIATQVPVLEPQLPGSVFDPIGHFTQPILHLPQPELPPHLPQPPEHSTPPHLNQHAVVSPPALHNALPQQPSRPSNRAAALPPKPARPPAVSPALTQTPLLPQPPMAQPPQVLLEDEEPPAPPLTSMQMQLYLQQLQKVQPPTPLLPSVKVQSQPPPPLPPPPHPSVQQQLQQQPPPPPPPQPQPPPQQQHQPPPRPVHLQPMQFSTHIQQPPPPQGQQPPHPPPGQQPPPPQPAKPQQVIQHHHSPRHHKSDPYSTGHLREAPSPLMIHSPQMSQFQSLTHQSPPQQNVQPKKQELRAASVVQPQPLVVVKEEKIHSPIIRSEPFSPSLRPEPPKHPESIKAPVHLPQRPEMKPVDVGRPVIRPPEQNAPPPGAPDKDKQKQEPKTPVAPKK

SEQ ID NO: 6 - DNA, 20 bp, synthetic construct
  source 1..20: mol_type = other DNA
aatcttttctgagcgcacct

SEQ ID NO: 7 - DNA, 20 bp, synthetic construct
  source 1..20: mol_type = other DNA
atcaaagtcagaagccacct

SEQ ID NO: 8 - protein, 46 aa, synthetic construct
SKLWLLKDRDLARQKEQERRRREAMVGTIDMTLQSDIMTMFENNFD

(19) United States
(12) Patent Application Publication
(10) Pub. No.: US 2024/0425555 A1
(43) Pub. Date: Dec. 26, 2024

(54) MUTANT BRD4 POLYPEPTIDES AND METHODS OF USE THEREOF

(71) Applicant: Northwestern University, Evanston, IL (US)

(72) Inventors: Ali Shilatifard, Evanston, IL (US); Lu Wang, Evanston, IL (US); Bin Zheng, Evanston, IL (US)

(21) Appl. No.: 18/749,183

(22) Filed: Jun. 20, 2024

Related U.S. Application Data
(60) Provisional application No. 63/509,145, filed on Jun. 20, 2023.

(51) Int. Cl. C07K 14/47; C12N 9/12; C12N 15/85; C12Q 1/48

(52) U.S. Cl. CPC C07K 14/47; C12N 9/1247; C12N 15/85; C12Q 1/485; G01N 2333/91255

(57) ABSTRACT

The present invention provides engineered BRD4 polypeptides comprising deletions of one or more of bromodomain 1 (BD1), bromodomain 2 (BD2), and extra terminal domain (ET). Also provided are polynucleotides encoding the engineered BRD4 polypeptides, and constructs and vectors comprising the polynucleotides. Also provided are methods for using the engineered BRD4 polypeptides to assess the effects of candidate BRD4 C-terminal specific inhibitors on Pol II pausing.

### CROSS-REFERENCE TO RELATED APPLICATIONS

This application claims the benefit of and priority to U.S. Provisional Application No. 63/509,145 filed on Jun. 20, 2023. The content of which is incorporated by reference in its entirety.

### STATEMENT REGARDING FEDERALLY SPONSORED RESEARCH

This invention was made with government support under grant number CA197569 awarded by the National Institutes of Health. The government has certain rights in the invention.

### REFERENCE TO A SEQUENCE LISTING

This application is being filed electronically via EFS-Web and includes a Sequence Listing in .xml format. The contents of the electronic sequence listing (702581.02517.xml; Size: 11,944 bytes; and Date of Creation: Jun. 20, 2024) is herein incorporated by reference in its entirety.

### BACKGROUND

Transcription catalyzed by RNA Polymerase II is tightly regulated at the distinct stages of initiation, pausing, elongation and termination. Pol II promoter-proximal pausing is a prominent feature at the majority of the genes in human cells, and the positive transcription elongation factor (P-TEFb), which consists of the catalytic subunit cyclin-dependent kinase 9 (CDK9) and the regulatory subunit Cyclin T, is an essential complex that regulates the release of promoter-proximal paused Pol II into elongation in gene bodies. Mechanistic studies have revealed that PTEFb mainly phosphorylates the serine residues at positions 2 and 5 of the heptapeptide repeats (52, in mammals) within the C-terminal domain (CTD) of RPB1, the main subunit of Pol II. PTEFb has also been shown to participate in multiple complexes, including the active BRD4-PTEFb complex, the active Super Elongation Complex (SEC) and the inactive 7SK-HEXIM complex, in which a majority of PTEFb is sequestered. The inventors of the present application have previously demonstrated that PTEFb may be recruited to Pol II by either BRD4 under normal conditions or by the SEC under stress conditions such as heat shock.

Bromodomain-containing protein 4 (BRD4) is a member of the bromodomain and extra terminal domain (BET) protein family, which also includes bromodomain-containing protein 2 (BRD2), bromodomain-containing protein 2 (BRD3) and bromodomain testis-specific protein (BRDT). Each BET family member has two tandem bromodomains, which can bind with high affinity to acetylated lysines within histone tails, and an extra terminal (ET) domain, which can interact with multiple transcription factors. However, only BRD4 and BRDT have C-terminal regions that can interact with PTEFb. Histone acetylation, especially the extensively studied H3K27ac, is a marker of active chromatin. Thus, a dominant model in the field holds that BRD4 bound to H3K27ac at promoters or enhancers recruits PTEFb to release paused Pol II, promoting transcription. In accordance with this model, bromodomain inhibitors have been developed and are now widely used both in research and in clinical trials; JQ1 is the most prominent example of a bromodomain inhibitor compound. Proteolysis targeting chimera (PROTAC) versions of JQ1 such as dBET6 further strengthen the impact of BRD4 targeting by inducing BRD4 degradation, but the effects of BRD4 depletion by these bromodomain inhibitor-based PROTACs differ strikingly from those of the inhibitors themselves. Some attempts to explain these differences focus on incomplete displacement of BRD4 from chromatin after JQ1 treatment. Moreover, accumulating evidence calls in to question the assumed requirement for H3K27ac marks in transcriptional regulation. Furthermore, recent studies and ongoing work increasingly suggest that histone acetylation may not be instructive to transcription in general, as complementation with catalytic-dead CBP and P300 mutants can restore gene expression just as well as their wild type, catalytic-active counterparts. Therefore, further investigation of BRD4 interaction with PTEFb and the mechanisms of BRD4-mediated release of Pol II pause is desired.

### SUMMARY OF THE INVENTION

In an aspect, an engineered bromodomain-containing protein 4 (BRD4) polypeptide comprising a deletion of at least one of bromodomain 1 (BD1), bromodomain 2 (BD2), and extra terminal domain (ET) is provided. The engineered BRD4 polypeptide may comprise a deletion of each of BD1, BD2, and ET. The polypeptide may comprise a C-terminal fragment of SEQ ID NO: 1 or SEQ ID NO: 2. The polypeptide may consist of the C terminal fragment of SEQ ID NO: 1 or SEQ ID NO: 2.

In another aspect, a polynucleotide encoding the engineered BRD4 polypeptide is provided.

In another aspect, a construct comprising the polynucleotide encoding the engineered BRD4 polypeptide operably linked to a promoter is provided. The promoter may be an inducible promoter. The construct may further comprise a sequence encoding a protein tag. The tag may be a Flag tag or a GFP tag.

In another aspect, a vector comprising the polynucleotide encoding the engineered BRD4 polypeptide is provided.

In another aspect, a cell comprising the engineered BRD4 polypeptide is provided. The cell may be depleted of endogenous BRD4.

In another aspect, an engineered bromodomain-containing protein 4 (BRD4) polypeptide comprising a deletion of a C-terminal fragment of SEQ ID NO: 1 or SEQ ID NO: 2 is provided.

In another aspect, a cell comprising the engineered polypeptide comprising a deletion of a C-terminal fragment of SEQ ID NO: 1 or SEQ ID NO: 2 is provided.

In another aspect, a kit comprising the engineered BRD4 polypeptide comprising a deletion of at least one of BD1, BD2, and ET, and further comprising a second engineered BRD4 polypeptide comprising a deletion of a C terminal fragment of SEQ ID NO: 1 or SEQ ID NO: 2 is provided.

In another aspect, a method for identifying a candidate agent as a BRD4 C-terminal specific inhibitor is provided, the method comprising: (a) measuring Pol II pausing in a first population of cells, wherein the first population of cells comprises the cell comprising the engineered polypeptide comprising a deletion of at least one of BD1, BD2, and ET, wherein the cell is depleted of endogenous BRD4; (b) contacting the first population of cells with the candidate agent; and (c) measuring Pol II pausing in the contacted first population of cells, wherein an increase in measured Pol II pausing in step (c) compared to measured Pol II pausing in step (a) identifies the candidate agent as a BRD4 C-terminal specific inhibitor.

The method may further comprise: (d) measuring Pol II pausing in a second population of cells, wherein the second population of cells comprises the cell comprising the engineered bromodomain-containing protein 4 (BRD4) polypeptide comprising a deletion of a C-terminal fragment of SEQ ID NO: 1 or SEQ ID NO: 2; wherein the cell is depleted of endogenous BRD4; (c) contacting the second population of cells with the candidate BRD4 C-terminal specific inhibitor; and (f) measuring Pol II pausing in the contacted second population of cells, wherein similar or reduced measured Pol II pausing in step (f) compared to measured Pol II pausing in step (d) further identifies the candidate agent as a BRD4 C-terminal specific inhibitor.

Pol II pausing may be measured by at least one of a cell growth assay and a Pol II-DNA binding assay. The first population of cells and the second population of cells may be DLD1 cells or NCIH2009 cells.

In another aspect, a method for increasing RNA polymerase II (Pol II) pause release in a cell is provided, the method comprising introducing the engineered BRD4 polypeptide comprising a deletion of at least one of bromodomain 1 (BD1), bromodomain 2 (BD2), and extra terminal domain (ET) into the cell.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIGS. 1A-1H collectively demonstrate that BRD4 degradation causes stronger Pol II pausing than inhibiting the bromodomains. FIG. 1A illustrates the updated BRD4-IAA7 degron line. The F-box protein AtAFB2 was integrated into the C terminus of the BRD4 locus under the control of an independent promoter. Western blot showing the acute depletion of BRD4 by auxin (500 uM) or dBET6 (250 nM) treatment but not JQ1 (1 uM) in the BRD4-IAA7 degron line. Cells were treated with auxin for 2 or 3 h, or with JQ1 or dBET6 for 3 h. These treatment concentrations/durations are relevant to all of FIG. 1. FIG. 1B illustrates the genome browser track examples of total Pol II ChIP-seq signal at the representative genes BRD2, PRSS22 and HSPA8 upon auxin treatment (0, 3 or 5 h), JQ1 or dBET6 treatment (3 h). FIG. 1C illustrates the heatmap showing the genome-wide Pol II occupancy profile upon auxin treatment and the profile of fold change in Pol II occupancy compared to control, in which the differential between promoter (high occupancy, red) and gene body (low occupancy, blue) indicates paused Pol II. The gene list (N=6481) in this analysis is used throughout the study (except for FIGS. 3H-J). FIG. 1D illustrates the estimated Cumulative Density Function (ECDF) of pause release ratios (PRR, reported as log 2 fold change) for auxin treatment vs untreated control. The PRR is the ratio of Pol II occupancy within gene bodies to its occupancy at promoters. A leftward shift of the curve indicates an increase in the frequency and/or duration of promoter-proximal pausing, while a rightward shift indicates reduced pausing and/or more efficient release into gene bodies. FIG. 1E illustrates the heatmap showing the genome-wide Pol II occupancy profile upon JQ1 or dBET6 treatment and the profile of fold change in occupancy vs control. FIG. 1F illustrates ECDF of the log 2PRR upon JQ1 or dBET6 treatment. FIG. 1G illustrates the boxplot of the log 2PRR for auxin, JQ1 and dBET6 treatment. FIG. 1H illustrates the PCA analysis of the log 2PRR for auxin, JQ1 and dBET6 treatment.

FIGS. 2A-2H collectively demonstrate that BRD4 bromodomains are dispensable for Pol II pause release. FIG. 2A illustrates the FLAG-tagged, Dox-inducible BRD4 mutant constructs, relevant to all of FIG. 2 (vector: TetOn-NLS-3xFLAG). The location of the epitope for the commercial BRD4 antibody (CST) is indicated. FIG. 2B illustrates the western blot showing BRD4 mutant construct expression in the same conditions used for subsequent rescue experiments: auxin-treated (3 h) BRD4-IAA7 cells, 2d after Dox treatment (50 nM). Blot was probed for BRD4 using antibodies against a C-terminal epitope (see A) and the N-terminal FLAG tags. FIG. 2C illustrates the track examples showing the total Pol II ChIP-seq signal at the BRD2, PRSS22 and HSPA8 loci for the rescue experiment, in which auxin-induced endogenous BRD4 depletion is complemented by Dox-induced mutant construct expression. FIG. 2D illustrates the heatmaps showing the genome-wide Pol II occupancy and fold change in occupancy for mutant constructs vs GFP control in the rescue experiment. FIG. 2E illustrates ECDF comparison of log 2PRR in the rescue experiment showing ΔCTM and BRD4S mutant constructs clustered with auxin-treated (BRD4 depleted) GFP control, while FL, ΔBD and ΔET constructs are clustered with the untreated GFP control. FIG. 2F illustrates the boxplot comparison of log 2PRR in the rescue experiment. FIG. 2G illustrates the track examples of the Pol II Ser2P ChIP-seq signal at the PRSS22 and HSPA8 loci, showing loss of Pol II Ser2P occupancy due to auxin-induced endogenous BRD4 depletion that is rescued by Dox-induced expression of the FL or ΔBD but not ΔCTM mutant constructs. FIG. 2H illustrates the heatmaps showing the genome-wide Ser2P occupancy under the same rescue experiment conditions.

FIGS. 3A-3J collectively demonstrate that bromodomain dispensability is conserved across cell lines. FIG. 3A illustrates the western blot showing degradation of endogenous BRD4 (V5) but not the bromodomain-less BRD4 mutant (FLAG) upon auxin or dBET6 treatment in the BRD4-IAA7 DLD1 degron line. FIG. 3B illustrates the track examples of the total Pol II ChIP-seq signal at the BRD2, PRSS22 and HSPA8 loci after dBET6 treatment and rescue by bromodomain-less BRD4 in DLD-1 cells. FIG. 3C illustrates the heatmap showing the genome-wide Pol II occupancy and fold change in occupancy for the bromodomain-less mutant construct vs GFP control after dBET6 treatment in DLD-1. FIG. 3D illustrates ECDF of the log 2PRR from the total Pol II ChIP-seq for the dBET6 rescue experiment in DLD-1. FIG. 3E illustrates the boxplot of the log 2PRR from the total Pol II ChIP-seq for the dBET6 rescue experiment in DLD-1. FIG. 3F illustrates the western blot showing the depletion of endogenous BRD4 and BRDT, but not the bromodomain-less mutant, by dBET6 treatment (3 h) in NCIH2009 cells. FIG. 3G illustrates track examples of the total Pol II ChIP-seq signal upon dBET6 treatment (3 h) and rescue by bromodomain-less BRD4 in NCIH2009 cells. FIG. 3H illustrates the heatmap showing the Pol II occupancy profiles and corresponding fold changes across the 1565 genes for which dBET6 causes strong pausing (2-fold reduction of Pol II at gene bodies and 2-fold reduction of PRR), rescued by bromodomain-less BRD4 in NCIH2009. FIG. 3I illustrates ECDF of the log 2PRR from the Pol II ChIP-seq for the dBET6 rescue experiment in NCIH2009 (N=1565). FIG. 3J illustrates the boxplot of the log 2PRR from the Pol II ChIP-seq for the dBET6 rescue experiment in NCIH2009 (N=1565).

FIGS. 4A-4H collectively demonstrate that BRD4 C-terminal fragment interacts with PTEFb and rescues Pol II pause release. FIG. 4A illustrates the GFP-tagged FL BRD4 and BRD4 C-terminus constructs (vector: TetOn-NLS-GFP, Cs: short C terminus). FIG. 4B illustrates GFP IP-MS results showing peptide counts for the bait (BRD4) and PTEFb complex components (CCNT1 and CDK9) in two replicates after Dox (50 nM) induced expression of Vector, CTM, CsΔCTM or Cs mutant constructs for 2 days. Note: the 0 bait peptide count for the CTM construct is probably due to impaired peptide alignment, as the CTM construct is itself a (GFP-tagged) peptide of only 37 residues. FIG. 4C illustrates the scatter plot showing the log 10Qvalue vs the enrichment over Vector (calculated by log 10FC of peptide counts+1) for the BRD4-Cs interacting proteins in the second GFP IP-MS replicate. FIG. 4D illustrates the western blot validating the GFP IP-MS result using the elute from the second replicate. FIG. 4E illustrates the track examples for total Pol II ChIP-seq signal at the PRSS22 and HSPA8 gene loci upon auxin treatment and rescue by different GFP-tagged mutants. FIG. 4F illustrates the heatmap showing the genome-wide Pol II occupancy profile and the corresponding fold changes for the GFP-tagged rescue experiment. FIG. 4G illustrates the track examples for Ser2P ChIP-seq signal at the BRD2 and HSPA8 gene loci upon auxin treatment and rescue by different GFP-tagged mutants. FIG. 4H illustrates the heatmap showing the genome-wide Pol II occupancy profile and the corresponding fold changes for the GFP-tagged rescue experiment.

FIGS. 5A-5J collectively illustrate the identification of a distinct BRD4-PTEFb population, which is active but not histone acetylation-bound. FIG. 5A illustrates the track examples for AID ChIP-seq in the BRD4-AID line and CCNT1 ChIP-seq in the BRD4-IAA7 line with/without auxin treatment (3 h). H3K27ac ChIP-seq signal in the BRD4-AID line is shown for comparison. FIG. 5B illustrates the heatmap showing the genome-wide BRD4 and CCNT1 occupancy profile and the corresponding fold change upon auxin treatment for the conditions in A. Genes ranked by BRD4 signal in control. FIG. 5C illustrates the scatter plot showing the relative promoter density of CCNT1 vs BRD4, calculated by the log 2RPKM of the ChIP-seq signals at the regions flanking (±1 Kb) the Pol II pausing sites. FIG. 5D illustrates the track examples for GFP ChIP-seq signal upon induction of GFP-tagged BRD4-FL or the indicated mutants (left). Heatmap showing the genome-wide fold changes of the GFP-tagged BRD4 mutants relative to the vector (right). The endogenous BRD4 was depleted by auxin treatment (3 h) in all samples. Genes are ranked by GFP signal in the GFP-BRD4-FL condition. FIG. 5E illustrates the track examples for CCNT1 ChIP-seq signal upon endogenous BRD4 depletion and rescue with indicated GFP-tagged mutants (left). Heatmap showing the genome-wide CCNT1 fold changes (right). Genes are ranked by CCNT1 signal in the untreated vector control. FIG. 5F illustrates the track examples for the CCNT1 ChIP-seq signal upon induction of the FLAG-tagged, CTMless BRD4 mutants by Dox treatment for 2 days (left). Heatmap showing the genome-wide profile of fold changes in CCNT1 occupancy (right). Genes are ranked by CCNT1 signal in the GFP control. FIG. 5G illustrates the track examples for the Ser2P ChIP-seq from the same samples in F (left). Heatmap showing the corresponding genome-wide Ser2P occupancy profile (right). FIG. 5H illustrates the track examples for the CCNT1 ChIP-seq in a series concentration of JQ1 and 250 nM dBET6 treatment for 3 h in the BRD4-IAA7 cells (left). Heatmap showing the corresponding genome-wide CCNT1 fold changes relative to DMSO treatment (right). Genes are ranked by CCNT1 signal in the DMSO control. FIG. 5I illustrates the track examples for the Ser2P ChIP-seq from the same JQ1 or dBET6-treated samples in H. FIG. 5J illustrates the heatmap showing the corresponding genome-wide Ser2P occupancy profile.

FIGS. 6A-6H collectively demonstrate that the BRD4 C-terminus stabilizes CyclinT1 in the BRD4-PTEFb complex. FIG. 6A illustrates the western blot of GFP IP showing the PTEFb complex components associated with BRD4 upon dCDK9 (2.5 uM) treatment for 3 h. GFP-tagged BRD4-FL was induced by Dox treatment for 2 days. FIG. 6B illustrates the GFP-tagged constructs: BRD4-FL, BRD4-C and BRD4-ΔCd (deletion of the C-terminal disordered region). FIG. 6C illustrates the western blot of GFP IP showing PTEFb complex components associated with BRD4 and its C terminal mutants. GFP-tagged BRD4-FL and mutant constructs were induced by Dox treatment for 2 days. Endogenous BRD4 was depleted in all samples by auxin treatment (3 h) prior to IP. FIG. 6D illustrates the track examples for the total Pol II ChIP-seq signal upon BRD4 depletion and rescue by BRD4-ΔCd. FIG. 6E illustrates the heatmap showing the genome-wide Pol II occupancy and the corresponding fold changes for the rescue experiment in D. FIG. 6F illustrates ECDF showing the log 2PRR for the Pol II ChIP-seq for the rescue experiment in D. FIG. 6G illustrates the boxplot showing the log 2PRR for the Pol II ChIP-seq for the rescue experiment in D. FIG. 6H illustrates the AlphaFold-predicted structure for CTM of BRD4 and the PTEFb complex (344 CDK9 N-terminal residues, 293 N-terminal CCNT1 residues and 37 C-terminal BRD4 residues were used for the computational prediction).

FIG. 7 illustrates the model for transcriptional regulation by a distinct layer of histone acetylation-unbound BRD4-PTEFb complex. Under normal cellular conditions, the majority of BRD4 molecules are associated with chromatin through binding to acetylated histone tails, and the PTEFb complex is recruited by its interaction with the C terminal of BRD4 to form the acetylation-bound “layer” of BRD4-PTEFb. A small portion of the remaining BRD4-PTEFb complex interacts with the Pol II CTD regardless of histone acetylation, forming a critical layer of BRD4-PTEFb that phosphorylates the Ser2 and Ser5 positions of the CTD heptapeptide repeats (Panel 1). BRD4 depletion achieved either by auxin in the BRD4 degron cells or dBET6 treatment results in genome-wide Pol II pausing (Panel 2). The acetylation-bound BRD4-PTEFb layer is displaced from the chromatin when cells are treated with the bromodomain inhibitor JQ1, but the acetylation-independent layer of BRD4-PTEFb can still function to release Pol II without histone recognition-mediated chromatin association (Panel 3). In the absence of acetylation (e.g., acetylation deposited by CBP/GCN5), the acetylation-independent, Pol II-bound BRD4-PTEFb complex retains the ability in releasing Pol II (Panel 4).

FIGS. 8A-8E. collectively illustrate the differences in Pol II pausing induced by BRD4 depletion and bromodomain inhibition, related to FIG. 1. FIG. 8A illustrates the western blot showing the expression of HA-tagged AtAFB2 and the auxin-induced degradation of V5-tagged BRD4 using anti-tag antibodies. FIG. 8B illustrates the western blot comparing the BRD4 protein expression level and degradation kinetics of the previously published BRD4-AID degron to those of the BRD4-IAA7 degron used in this study 12. Note: in FIGS. 8A and 8B, the BRD4-IAA7 clone used is D4 (Hygromycin selected); it is G3 (Neomycin selected) otherwise in the study. FIG. 8C illustrates the scatter plot of the gene-by-gene PRR correlations between auxin treatment durations and between JQ1 and auxin, dBET6 and auxin, or JQ1 and dBET6 treatment (normalized to corresponding vehicles). FIG. 8D illustrates the track examples of Pol II ChIP-seq signal at the MYC locus upon auxin treatment (0, 3 or 5 h), JQ1 or dBET6 treatment (3 h). FIG. 8E illustrates the track examples of Pol II ChIP-seq signal at the HSPA8 and GAPDH loci upon NVP-2 treatment (2 h) and corresponding heatmaps showing Pol II occupancy profiles and fold change in occupancy vs control upon NVP-2 treatment.

FIGS. 9A-9C collectively illustrate BRD4 depletion and rescue by different mutants, related to FIG. 2. FIG. 9A illustrates the tracks of the total Pol II ChIP-seq signal at the BRD4 gene locus in the rescue experiment with the arrows indicating the loss of exonic signal corresponding to mutant sequence. FIG. 9B illustrates the tracks of the total Pol II ChIP-seq signal at the MYC gene locus as well as the genebody zoom in for different mutant rescue upon auxin treatment. FIG. 9C illustrates the plate images showing cell growth over the time course of BRD4 depletion and mutant complementation.

FIGS. 10A-10G collectively illustrate bromodomain-less BRD4 expression in NCIH2009 cells and the screen for a minimal CTM-containing fragment in DLD-1 cells, related to FIG. 3. FIG. 10A illustrates the tracks comparing the Pol II ChIP-seq signal at the BRDT gene locus in DLD-1 and NCIH2009 cells. FIG. 10B illustrates the fluorescent images comparing the expression of GFP control in the DLD-1 and NCIH2009 cells. FIG. 10C illustrates the heatmap showing the genome-wide Pol II occupancy and the corresponding fold changes for the dBET6 treatment and rescue by ΔBD complementation in NCIH2009 cells. FIG. 10D illustrates the FLAG-tagged, bromodomain-less BRD4 mutant constructs, with western blot showing protein expression upon Dox treatment (2d) in BRD4-IAA7 cells. FIG. 10E illustrates the track examples of the total Pol II ChIP-seq signal upon auxin treatment in BRD4-IAA7 cells expressing the FLAG-tagged mutants in D. FIG. 10F illustrates the heatmap of the genome-wide Pol II occupancy profile from the Pol II ChIP-seq in E and the corresponding fold changes in occupancy relative to DBD. FIG. 10G illustrates the boxplot showing the log 2PRR for the conditions in E.

FIGS. 11A-11I collectively demonstrate that BRD4 C terminal fragments interact with PTEFb and release paused Pol II, related to FIG. 4. FIG. 11A illustrates the western blot showing the expression of GFP-tagged FL BRD4 or the CTM, CsΔCTM or Cs mutant constructs upon Dox induction (2d.) (Vector: TetOn-NLS-GFP). FIG. 11B illustrates ECDF showing the log 2PRR from the Pol II ChIP-seq for the rescue experiment in FIG. 4E. FIG. 11C illustrates the boxplot showing the log 2PRR from the Pol II ChIP-seq for the rescue experiment in FIG. 4E. FIG. 11D illustrates the Metagene plot showing Pol II occupancy around the TES as a reference point, for the Pol II ChIP-seq conditions in FIG. 4E. FIG. 11E illustrates the track examples showing the total Pol II ChIP-seq signal for BRD4 depletion and rescue with indicated mutants. FIG. 11F illustrates the heatmap showing the genome-wide Pol II occupancy profiles and the corresponding fold changes for the conditions in F. FIG. 11G illustrates the Metagene plot showing the Pol II occupancy profile, around the TES as a reference point, from the Pol II ChIP-seq for conditions in F. FIG. 11H illustrates ECDF showing the log 2PRR from the Pol II ChIP-seq for conditions in F. FIG. 11I illustrates the boxplot showing the log 2PRR from the Pol II ChIP-seq for conditions in F.

FIGS. 12A-12F collectively demonstrate that genome-wide PTEFb binding relies on BRD4 association with histone acetylation, related to FIG. 5. FIG. 12A illustrates the track examples comparing the ChIP-seq signals for Pol II, CCNT1 and CDK9 in the same samples of auxin-treated (3 h) BRD4-IAA7 cells. FIG. 12B illustrates the heatmap showing the genome-wide CDK9 occupancy and the corresponding fold change upon auxin treatment for the condition in A. Genes are ranked by CDK9 signal in control. FIG. 12C illustrates the track example for GFP ChIP-seq upon induction of GFP-tagged BRD4-FL or the indicated mutants (left). Heatmap showing the genome-wide fold changes of the GFP-tagged BRD4 FL and mutant constructs relative to the vector (right). The endogenous BRD4 is depleted by auxin treatment for 3 h across all samples. Genes ranked by GFP signal in the GFP-BRD4-FL condition. FIG. 12D illustrates the track examples of Pol II ChIP-seq signal upon Dox induction for the CTM-less BRD4 mutants. This experiment was carried out concurrently with the rescue experiment in FIG. 2. The GFP condition in FIGS. 2C and 2D was used here as a control. FIG. 12E illustrates the Metagene plot showing the Pol II occupancy for the conditions in F with a zoom in view around the TES as a reference point. FIG. 12F illustrates the heatmap showing the genome-wide Pol II occupancy profiles and the corresponding fold changes for the conditions in F.

FIGS. 13A-13C collectively demonstrate that BRD4 C-terminus stabilizes PTEFb and confers Pol II CDT phosphorylation activity, related to FIG. 6. FIG. 13A illustrates the GFP-tagged BRD4-FL, BRD4-C and two versions of BRD4-ΔCd (partial and total deletion of the C-terminal disordered region). FIG. 13B illustrates the track examples of the Ser2P ChIP-seq signal upon BRD4 depletion and rescue by the two versions of BRD4-ΔCd. This experiment was carried out concurrently with the rescue experiment in FIG. 4. The Vec−/+Auxin conditions in FIGS. 4G and 4H were used here as control. FIG. 13C illustrates the heatmap showing the genome-wide Ser2P occupancy for the conditions in B.

FIGS. 14A-14G. Identification of essential PTEFb binding sites in BRD4's C-terminus. FIG. 14A. Schematic showing the constructs of Dox inducible GFP-tagged BRD4 C terminus mutants with residue length indicated. Western blot of GFP IP for these two mutants upon Dox induction for 2 days showing both mutants can interact with PTEFb. FIG. 14B. Western blot for the endogenous BRD4 IP upon expression of BRD4 C terminus 46 amino acids in the BRD4-V5-IAA7 degron line showing decreased binding of PTEFb. Auxin treatment serves as a positive control for the loss of endogenous BRD4. FIG. 14C. Synthetic Biotin-labeled peptide of the BRD4 C terminus 46 residues (Bio-p46) was able to pull down PTEFb while the ability is largely impaired when FEE in the tail of the 46 residues were mutated to AAA (Bio-p46*). FIG. 14D. Western blot showing decreased binding of PTEFb to BRD4 when synthetic peptide of the BRD4 C terminus 46 residues (p46) was added to the cell lysate in prior to BRD4 IP. FIG. 14E. Schematic showing the sequence alignment of the 46 residues in the C terminus of BRD4 and BRDT as well as the alanine scanning strategy to mutate each site for 3 amino acids at one time. FIG. 14F. Comparison of the ability to pull down PTEFb by Bio-p46/Bio-p46* and Biotin-labeled BRDT C terminus 46 residues (Bio-pT46) as well as Biotin-labeled BRD4 C terminus 46 residues with each of the sites mutated to AAA as shown in E (Bio-p1 to p7). FIG. 14G. Western blot of GFP IP showing the amount of PTEFb binding to GFP-tagged BRD4 C terminus fragment of 340 residues (WT) and its mutants with each of the sites mutated to AAA as indicated in E (A-H).

FIGS. 15A-15E. FIG. 15A. Spectral counts for BRD4, CCNT1, and CDK9 from the GFP IP for the GFP-tagged BRD4 C terminus fragment of 340 residues (C) and 46 residues (C46) followed by mass spectrometry. FIG. 15B. Western blot validation for the mass spectrometry using the remaining GFP IP samples from one of the replicates. FIG. 15C. Western blot showing decreased binding of PTEFb to GFP-tagged 46 residues when synthetic peptide of the BRD4 C terminus 46 residues was added to the cell lysate in prior to GFP IP. FIG. 15D. Bio-p46 pull down assay showing the decreased binding of PTEFb to the Biotin-labeled BRD4 C terminus 46 residues when the cell lysate was pre-incubated with increasing concentration of p46. FIG. 15E. Quantitative curves of PTEFb signal intensity from western blot in D against increasing concentration of p46.

### DETAILED DESCRIPTION

The present disclosure provides mutant BRD4 polypeptides and methods for examining potential C-terminal-specific BRD4 inhibitors. Bromodomain-containing protein 4 (BRD4) is a protein that in humans is encoded by the BRD4 gene. Similar to other bromodomain and extra terminal domain (BET) family members, BRD4 contains two bromodomains that recognize acetylated lysine residues. BRD4 also has an extended C-terminal domain that has less sequence homology to other BET family members.

In the Examples, the inventors found the PTEFb-interacting C-terminal region of BRD4 to be essential and sufficient for release of Pol II pausing, a transcription halt following initiation and prior to elongation. A small, bromodomain-less C-terminal BRD4 fragment was sufficient to mediate release of paused Pol II in the absence of full-length BRD4. Mutant BRD4 polypeptides having only the C-terminal region or the other regions of BRD4 (e.g. bromodomains and extraterminal domain) may be used as controls to study the effectiveness of BRD4 inhibitors.

Pol II pause release is a critical regulation step in transcription. Disorders will lead to malfunction of the cellular processes. The benefit of using the bromodomain-less C-terminal BRD3 fragment is to improve transcription without delay caused by the process of histone acetylation.

Engineered polypeptides:

In a first aspect, provided herein are engineered BRD4 polypeptides. In embodiments, the polypeptide comprises deletions of at least one of bromodomain 1 (BD1), bromodomain 2 (BD2), and extra terminal domain (ET). The engineered polypeptide may comprise deletions of BD1 and BD2. The engineered polypeptide may comprise deletions of each of BD1, BD2, and ET. The engineered polypeptide may comprise or consist of a C-terminal fragment of SEQ ID NO: 1 or SEQ ID NO: 2.

In embodiments, the engineered polypeptide comprises a deletion of the C-terminal region of BRD4. The engineered polypeptide may consist of sequence having at least 50%, 60%, 70%, 80%, 85%, 90%, 95%, 98%, 99%, or 100% sequence identity to a polypeptide of SEQ ID NO: 1 or 2SEQ ID NO: 4 or SEQ ID NO: 5. The engineered polypeptide may comprise a deletion of a C-terminal fragment of SEQ ID NO: 1 or SEQ ID NO: 2.

The terms “polypeptide,” “protein,” and “peptide” are used interchangeably herein to refer to a series of amino acid residues connected by peptide bonds between the alpha-amino and carboxy groups of adjacent residues. Polypeptides include modified amino acids. Suitable polypeptide modifications include, but are not limited to, acylation, acetylation, formylation, lipoylation, myristoylation, palmitoylation, alkylation, isoprenylation, prenylation, amidation at C-terminus, glycosylation, glycation, polysialylation, glypiation, and phosphorylation. Polypeptides may also include amino acid analogs.

The engineered BRD4 polypeptides described herein may be derived from full-length polypeptides or fragments of a full-length polypeptide. As used herein, a “fragment” is a portion of a polypeptide that is identical in sequence to, but shorter in length than, the full-length polypeptide. For example, a fragment may comprise at least 5, 10, 15, 20, 25, 30, 40, 50, 60, 70, 80, 90, 100, 150, 250, or 500 contiguous amino acid residues of a full-length polypeptide. Fragments may be preferentially selected from certain regions of a polypeptide. A fragment may include an N-terminal truncation, a C-terminal truncation, or both an N-terminal and C-terminal truncation relative to the full-length polypeptide. Preferably, the BRD4 polypeptide fragments used with the present invention are functional fragments. As used herein, a “functional fragment” is a fragment that retains at least 20%, 40%, 60%, 80%, or 100% of the BRD4 activity of the corresponding full-length polypeptide.

The polypeptides described herein are “engineered,” meaning that they have been altered by the hand of man. Specifically, the engineered BRD4 polypeptides of the present invention have been altered to comprise a mutation. As used herein, the term “mutation” refers to a difference in an amino acid sequence relative to a reference sequence (e.g., the sequence of the wild-type polypeptide). Mutations include insertions, deletions, and substitutions of an amino acid relative to a reference sequence. An “insertion” refers to a change in an amino acid sequence that results in the addition of one or more amino acid residues. An insertion may add 1, 2, 3, 4, 5, 10, 20, 30, 40, 50, 60, 70, 80, 90, 100, 150, 200, or more amino acid residues to a sequence. A “deletion” refers to a change in an amino acid sequence that results in the removal of one or more amino acid residues. A deletion may remove 1, 2, 3, 4, 5, 10, 20, 50, 100, 200, or more amino acids residues from a sequence. A “substitution” refers to a change in an amino acid sequence in which one amino acid is replaced with a different amino acid. An amino acid substitution may be a conversative replacement (i.e., a replacement with an amino acid that has similar properties) or a radical replacement (i.e., a replacement with an amino acid that has different properties).

The engineered BRD4 of the present invention comprise one or more mutations relative to the corresponding wild-type polypeptide (i.e., the wild-type version of the same BRD4 polypeptide). The term “wild-type” is used to describe the non-mutated version of a polypeptide that is most typically found in nature.

In some embodiments, the engineered BRD4 polypeptide comprises a polypeptide or a functional fragment thereof having at least 50%, 60%, 70%, 80%, 85%, 90%, 95%, 98%, 99%, or 100% sequence identity to a polypeptide of SEQ ID NO: 1 or 2. “Percentage of sequence identity” is determined by comparing two optimally aligned sequences over a comparison window. The aligned sequences may comprise additions or deletions (i.e., gaps) relative to each other for optimal alignment. The percentage is calculated by determining the number of matched positions at which an identical nucleic acid base or amino acid residue occurs in both sequences, dividing the number of matched positions by the total number of positions in the window of comparison and multiplying the result by 100 to yield the percentage of sequence identity. Protein and nucleic acid sequence identities are evaluated using the Basic Local Alignment Search Tool (“BLAST”), which is well known in the art (Proc. Natl. Acad. Sci. USA (1990) 87:2267-2268; Nucl. Acids Res. (1997) 25:3389-3402). The BLAST programs identify homologous sequences by identifying similar segments, which are referred to herein as “high-scoring segment pairs”, between a query amino acid or nucleic acid sequence and a test sequence which is preferably obtained from a protein or nucleic acid sequence database. Preferably, the statistical significance of a high-scoring segment pair is evaluated using the statistical significance formula Proc. Natl. Acad. Sci. USA (1990) 87:2267-2268), the disclosure of which is incorporated by reference in its entirety. The BLAST programs can be used with the default parameters or with modified parameters provided by the user.

### Polynucleotides:

In a second aspect, provided is a polynucleotide encoding any of the engineered polypeptides disclosed herein. The terms “polynucleotide,” “oligonucleotide,” and “nucleic acid” are used interchangeably to refer a polymer of DNA or RNA. A polynucleotide may be single-stranded or double-stranded and may represent the sense or the antisense strand. A polynucleotide may be synthesized or obtained from a natural source. A polynucleotide may contain natural, non-natural, or altered nucleotides, as well as natural, non-natural, or altered internucleotide linkages (e.g., phosphoroamidate linkages, phosphorothioate linkages). The term polynucleotide encompasses constructs, vectors, plasmids, and the like. In some embodiments, the polynucleotide is complementary DNA (cDNA; i.e., synthetic DNA that has been reverse transcribed from a messenger RNA) or genomic DNA (i.e., chromosomal DNA from an organism). Those of skill in the art understand the degeneracy of the genetic code and that a variety of polynucleotides can encode the same polypeptide.

### Constructs:

In a third aspect, provided is a construct comprising a promoter operably linked to any one of the polynucleotides described herein. As used herein, the term “construct” refers a to recombinant polynucleotide, i.e., a polynucleotide that was formed by combining at least two polynucleotide components from different sources, natural or synthetic. For example, a construct may comprise the coding region of one gene operably linked to a promoter that is (1) associated with another gene found within the same genome, (2) from the genome of a different species, or (3) synthetic. Constructs can be generated using conventional recombinant DNA methods.

As used herein, the term “promoter” refers to a DNA sequence defines where transcription of a polynucleotide beings. RNA polymerase and the necessary transcription factors bind to the promoter to initiate transcription. Promoters are typically located directly upstream (i.e., at the 5′ end) of the transcription start site. However, a promoter may also be located at the 3′ end, within a coding region, or within an intron of a gene that it regulates. Promoters may be derived in their entirety from a native or heterologous gene, may be composed of elements derived from multiple regulatory sequences found in nature, or may comprise synthetic DNA. A promoter is “operably linked” to a polynucleotide if the promoter is positioned such that it can affect transcription of the polynucleotide.

The promoter used in the constructs described herein may be a heterologous promoter (i.e., a promoter that is not naturally associated with the BRD4 polynucleotide), an endogenous promoter (i.e., a promoter that is naturally associated with the BRD4 polynucleotide), or a synthetic promoter that is designed to function in a desired manner in a particular host cell. Suitable promoters for use with the present invention include, but are not limited to, constitutive, inducible, temporally regulated, developmentally regulated, chemically regulated, tissue-preferred, and tissue-specific promoters. In exemplary embodiments, the promoter is an doxycycline inducible promoter. In some cases, it may be advantageous to use a tissue-specific promoter or a developmental stage-specific promoter such that the construct will drive expression of the BRD4 polypeptide in a particular tissue or in a particular disease environment.

The construct may further comprise a sequence encoding a protein tag. The protein tag may be used for purification of the recombinant polypeptide by acting as a ligand in affinity purification (e.g., a FLAG tag or a His tag), to tag the recombinant polypeptide for identification (e.g. green fluorescence protein (GFP) or an antigen (e.g. an HA tag) that can be recognized by a labelled antibody), or to promote localization of the recombinant protein to a specific area of the cell (e.g. a nuclear localization signal (NLS).

### Vectors:

In a fourth aspect, provided is a vector comprising any one of the polynucleotides or constructs described herein. The term “vector” refers to a DNA molecule that is used to carry a particular DNA segment (i.e., a DNA segment included in the vector) into a host cell. Some vectors are capable of autonomous replication in a host cell (e.g., bacterial vectors that include an origin of replication and episomal mammalian vectors). Other vectors can be integrated into the genome of a host cell such that they are replicated along with the host genome (e.g., viral vectors and transposons). Vectors may include heterologous genetic elements that are necessary for propagation of the vector or for expression of an encoded gene product. Vectors may also include a reporter gene or a selectable marker gene. Suitable vectors include plasmids (i.e., circular double-stranded DNA molecules) and mini-chromosomes.

### Cells:

In a fifth aspect, provided is a cell comprising any one of the engineered polypeptides, polynucleotides, constructs, or vectors described herein. The cells may be eukaryotic or prokaryotic. Preferably, the cell is a mammalian cell. In exemplary embodiments, the cell is a DLD1 cell or a NCIH2009 cell.

### Kits:

In a sixth aspect, provided is a kit comprising any one of the engineered BRD4 polypeptides comprising a deletion of at least one of BD1, BD2, and ET described herein and any one of the engineered BRD4 polypeptides comprising a deletion of a C-terminal fragment of SEQ ID NO: 1 or SEQ ID NO: 2 described herein. The engineered BRD4 polypeptides may be provided in separate containers.

### Methods for Releasing Pol II Pausing:

In a seventh aspect, provided is a method for releasing RNA polymerase II (Pol II) pausing in a cell by introducing into the cell an engineered BRD4 polypeptide comprising a deletion of at least one of the BD1, BD2 and ET domains, a polynucleotide encoding the engineered BRD4 polypeptide, a construct comprising the polynucleotide, or a vector comprising the polynucleotide or construct. The engineered BRD4 polypeptide may comprise a deletion of all of the BD1, BD2 and ET domains. As used herein, “introducing” describes a process by which exogenous polypeptides or polynucleotides are introduced into a recipient cell. Suitable introduction methods are known in the art (e.g. transduction, transfection, transformation, etc.), and described herein.

### Methods for Identifying Candidate BRD4 Inhibitors:

In an eighth aspect, provided herein is a method for identifying candidate BRD4 inhibitors. The method comprises measuring Pol II pausing on a first population of cells, the cells comprising an engineered bromodomain-containing protein 4 (BRD4) polypeptide comprising a deletion of at least one of bromodomain 1 (BD1), bromodomain 2 (BD2), and extra terminal domain (ET), and is depleted of endogenous BRD4; contacting the cells with a candidate BRD4 C-terminal specific inhibitor; and measuring Pol II pausing in the contacted first population of cells, where an increase in measured Pol II pausing in the contacted cells compared to measured Pol II pausing in the cells in the absence of the candidate inhibitor identifies the BRD4 C-terminal specific inhibitor. The cells may lack endogenous BRD4.

The C-terminal specific inhibitor refers to a compound that binds to and/or inhibits the activity of the BRD4 C-terminal region.

The method may further comprise measuring Pol II pausing in a second population of cells, wherein the second population of cells expresses endogenous BRD4; contacting the second population of cells with the candidate BRD4 C-terminal specific inhibitor and measuring Pol II pausing in the contacted second population of cells, wherein an increase in measured Pol II pausing in the contacted second population of cells compared to measured Pol II pausing in the second population of cells in the absence of the candidate BRD4 C-terminal specific inhibitor further identifies the BRD4 C-terminal specific inhibitor.

The method may further comprise measuring Pol II pausing in a third population of cells, the cells depleted of endogenous BRD4 and comprising an engineered BRD4 polypeptide comprising a deletion of the C-terminal region (SEQ ID NO: 1 or SEQ ID NO: 2); contacting the third population of cells with the candidate BRD4 C-terminal specific inhibitor; and measuring Pol II pausing in the contacted third population of cells, wherein similar or reduced measured Pol II pausing in the contacted third population of cells compared to measured Pol II pausing in the third population of cells in the absence of the candidate BRD4 C-terminal specific inhibitor identifies the BRD4 C-terminal specific inhibitor.

The first and third populations of cells may comprise the engineered BRD4 polypeptide of SEQ ID NO: 1, 2, 4, or 5, a polynucleotide encoding the engineered polypeptide, a construct comprising the polynucleotide having a promoter and a protein tag (e.g. Flag tag or GFP tag), or a vector comprising the construct. The cell populations may be prepared by any suitable methods known in the art, including those described herein. The cell populations may be comprised of stable mutant cell lines. Each of the first, second, and third population cells may be DLD1 cells or NCIH2009. As used herein, the term “contacting” or “to contact” a cell population refers to adding to the cell culture media the candidate BRD4 C-terminal specific inhibitor.

Endogenous BRD4 depletion may be achieved by any suitable methods known in the art. In exemplary embodiments, the endogenous BRD4 depletion may achieved by contacting the cell with auxin or dBET6.

Pol II pausing may be measured by any techniques and methods known in the art. Pol II pausing may be measured by a cell growth assay, where increased cell growth indicates release of Pol II pausing. Cell growth may be assayed by any suitable methods known in the art, including those described herein. Pol II pausing may also be measured by a Pol II-DNA binding assay, e.g. ChIP-seq, as described herein.

### Methods for Inhibiting Endogenous BRD4

In a ninth aspect, provided herein is a method for inhibiting endogenous BDR4 chromatin binding in a subject in need thereof, the method comprising administering to the subject the peptide having at least 50%, 60%, 70%, 80%, 85%, 90%, 95%, 98%, 99%, or 100% sequence identity to a polypeptide of SEQ ID NO: 1 or 2.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1

### Introduction

The BET family protein BRD4 is considered the master regulator of RNA Polymerase II (Pol II) pause release. In the long-held model, BRD4 function relies on interactions of its tandem bromodomains with acetylated histone lysine residues to facilitate its recruitment to chromatin, where it forms the BRD4-PTEFb complex to phosphorylate Pol II's C terminal domain (CTD), initiating release of paused Pol II. In this study we evaluated the established model by scrutinizing the functions of individual BRD4 domains using genetic complementation after acute depletion of endogenous BRD4.

The rescue experiments described herein revealed that BRD4 participates in Pol II pause release independently of its bromodomains binding to acetylated histones. We tested this phenomenon in distinct human cell lines, gaining confidence in the broad mechanistic implication. A “layer” of BRD4-PTEFB is associated with chromatin through BRD4 bromodomain-mediated interactions with acetylated histones. However, our study reveals that when we remove this layer, either by disrupting the interaction of BRD4 bromodomains with acetylation or through replacement of endogenous BRD4 with bromodomain-less versions, Pol II pause release remains functional genome-wide. Our genetic and molecular studies have also resulted in the identification of a novel BRD4 region necessary for Pol II pause release that functions independently of any bromodomain-mediated chromatin association. Altogether, this study introduces an important and possibly singular mechanism of histone acetylation-independent transcriptional regulatory function for BRD4, providing a new basis for pharmaceutical modulation of BRD4-PTEFb activity to control transcription.

### Results

### Broad Genome-Wide Changes in Pol II Occupancy Upon BRD4 Depletion but not Bromodomain Inhibition.

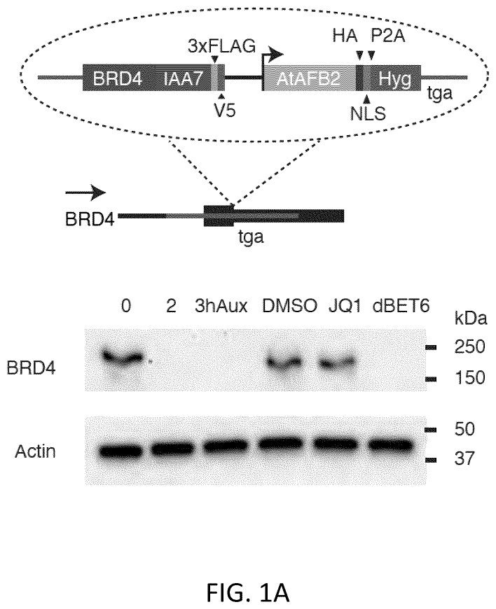

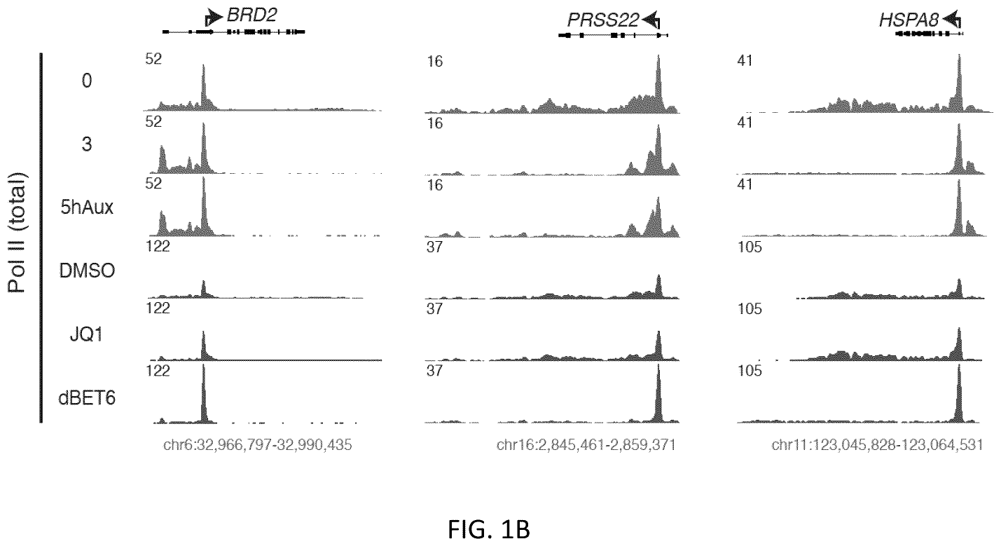

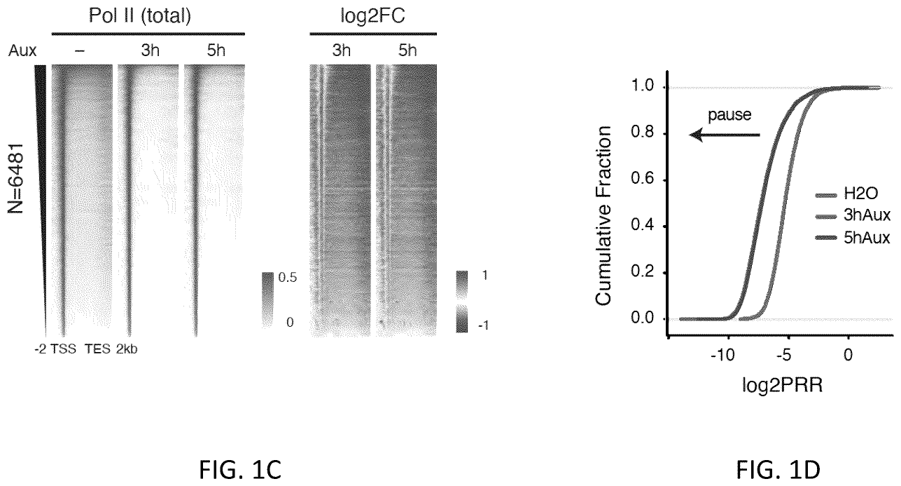

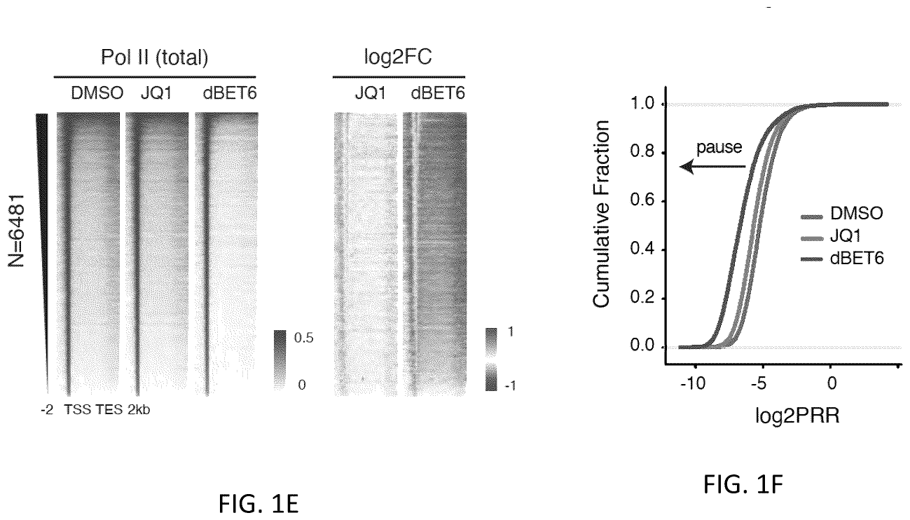

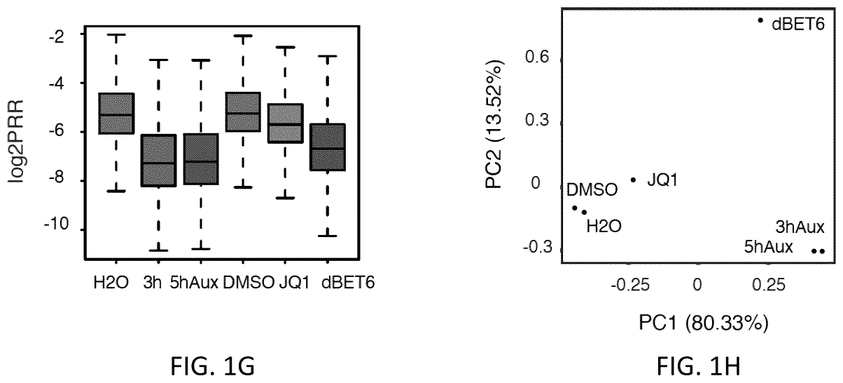

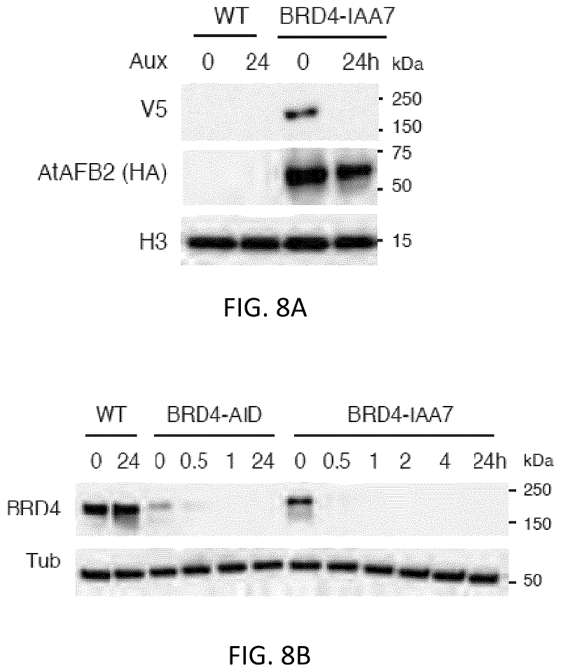

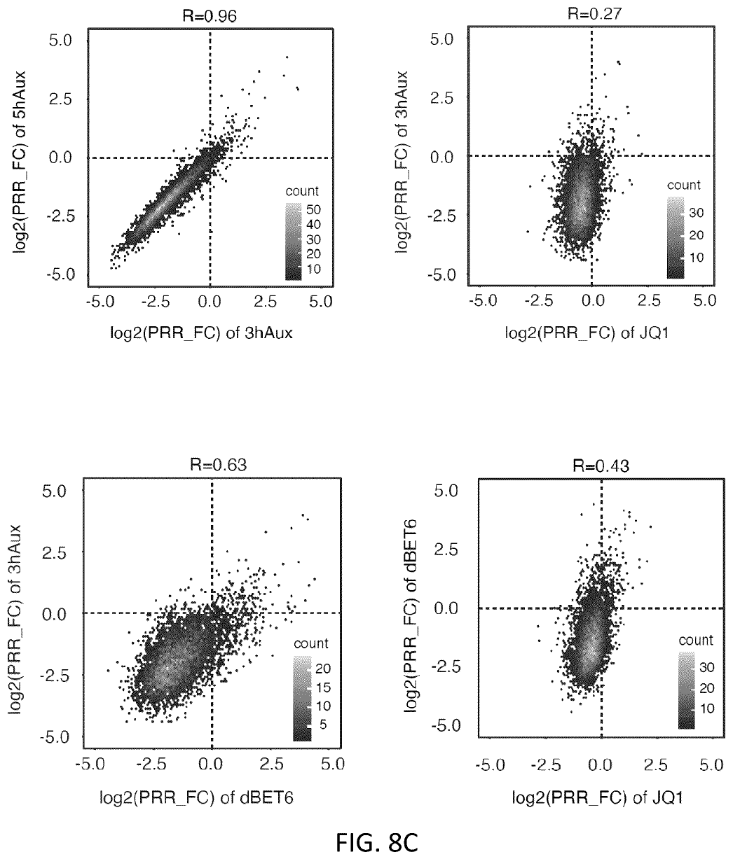

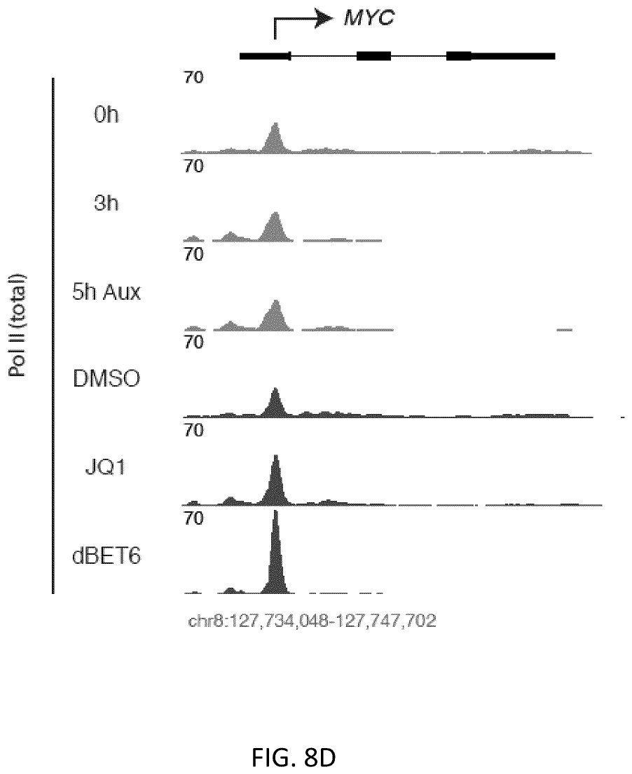

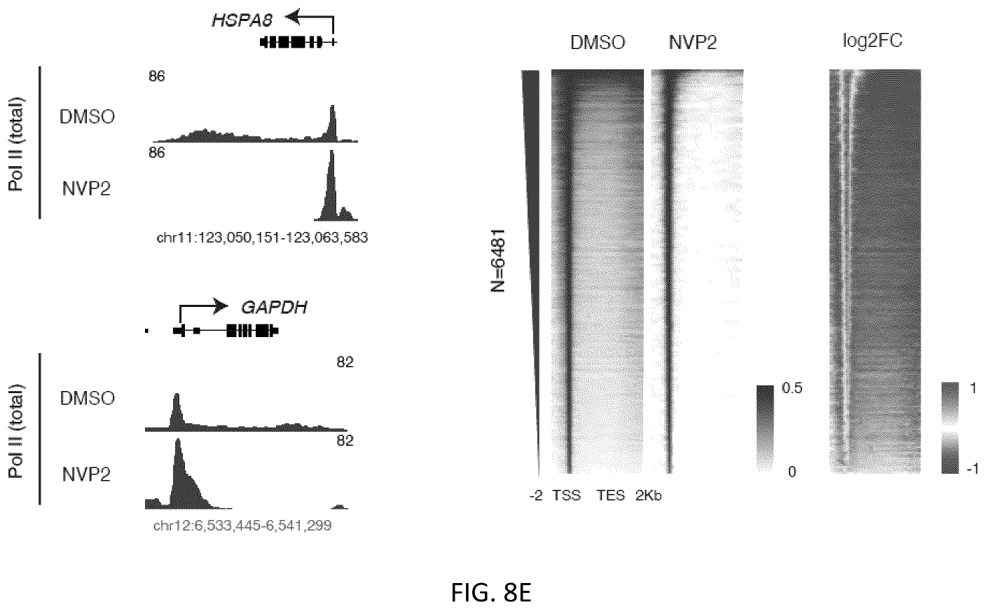

It has been demonstrated, both by RNA-seq and by measurement of nascent RNA associated with Pol II on chromatin (NET-seq), that the effect of the BET protein family inhibitor JQ1 differs from that of its PROTAC version, dBET6: while JQ1 bromodomain disruption has only a modest effect on Pol II pausing, dBET6-induced degradation of BRD2, BRD3, BRD4 and BRDT proteins leads to a dramatic, genome-wide increase in paused Pol II at promoters.²¹ To better understand the molecular mechanism underlying this disparity, we compared the effects of JQ1 or dBET6 treatment to that of acute and specific depletion of BRD4, the BET family member implicated in the regulation of Pol II pause release^{12,28,29}. To specifically deplete BRD4 without affecting other BET proteins, we generated an updated BRD4 degron line by modifying the published miniIAA7 degron system (hereafter BRD4-IAA7) to reduce basal degradation seen with AID degron-tagged BRD4³⁰ (FIGS. 1A and 8A). BRD4 was rapidly degraded upon auxin treatment in our updated line, and indeed basal degradation appears to be reduced compared to the BRD4-AID line (FIG. 8B). Compared to auxin treatment in BRD4-IAA7, dBET6 treatment degrades BRD4 to a similar extent, while JQ1 treatment does not, as expected (FIG. 1A). We then performed Pol II ChIP-seq upon auxin, JQ1 or dBET6 treatment in BRD4-IAA7 cells to assess the impacts of BRD4 degradation vs bromodomain inhibition on the Pol II occupancy profile. As expected, dBET6 treatment phenocopied auxin treatment, inducing pausing at the majority of Pol II-transcribed genes, while JQ1 induced little to no pausing (FIGS. 1B, IC, and IE). As shown by Estimated Cumulative Density Fraction (ECDF) and boxplot representations, as well as PCA analysis of the pause release ratios (PRR, reported as log 2 fold change), JQ1 treatment did not recapitulate the genome-wide Pol II pausing effect seen upon auxin or dBET6 treatment (FIGS. 1D and 1F-1H). As shown by scatter plot representation, the gene-by-gene log 2PRR correlation with auxin treatment was much stronger for dBET6 than for JQ1, for which the change in PRR was poorly correlated with that of auxin treatment on a gene-by-gene basis (FIG. 8C). An effect of JQ1 on pausing was notable at super enhancer-controlled genes such as Myc^{22,31} (FIG. 8D). Since BRD4 is known to interact with PTEFb to phosphorylate Pol II^{32,33}, we also tested the effect of the potent CDK9 inhibitor NVP2³¹ in DLD1 cells and found that CDK9 inhibition also induced strong Pol II pausing genome-wide (FIG. 8E). Taken together, these data demonstrated that bromodomain inhibition was not sufficient to abolish BRD4's participation in Pol II pause release, suggesting that PTEFb might release Pol II through a BRD4-dependent but bromodomain-independent mechanism.

### Bromodomains of BRD4 are Dispensable for Pol II Pause Release in Living Cells

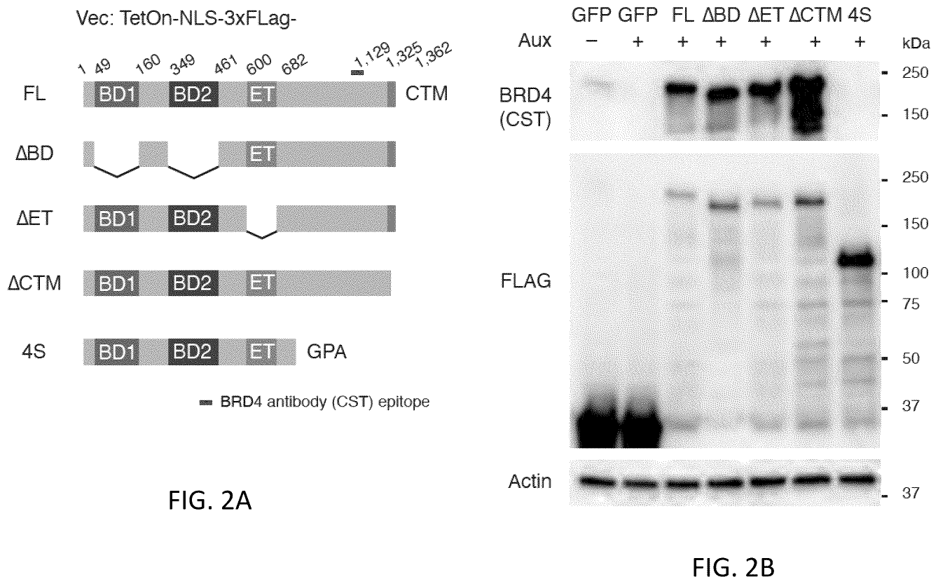

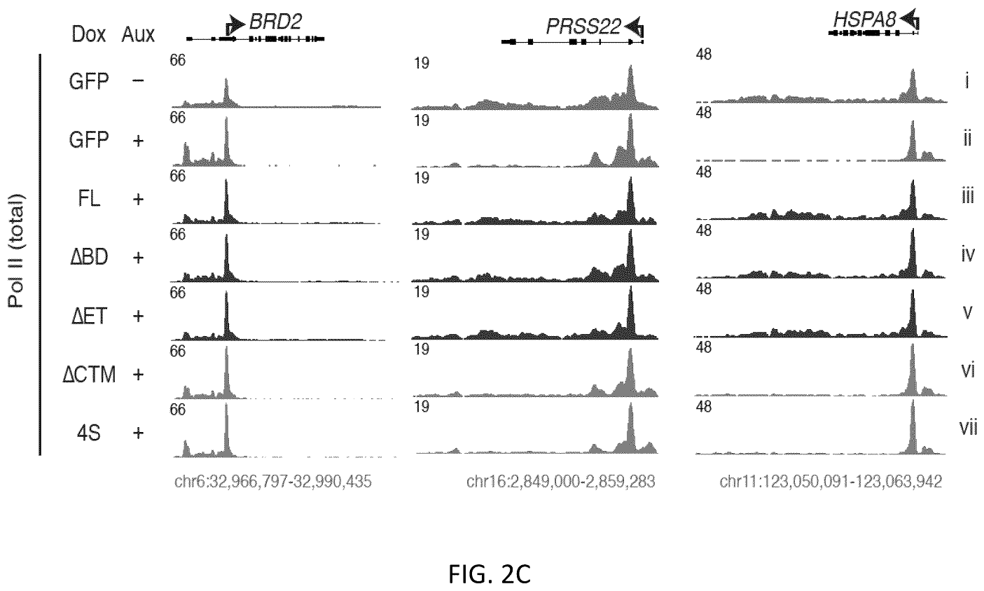

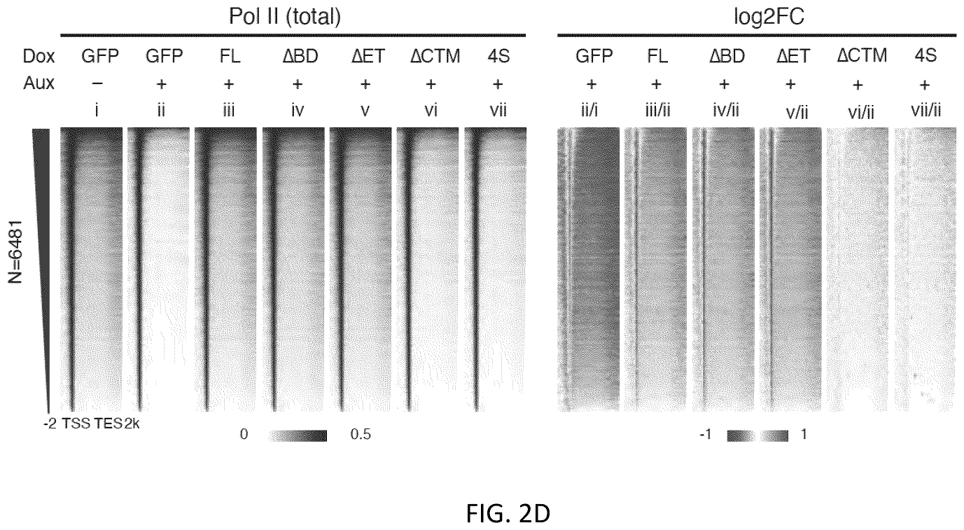

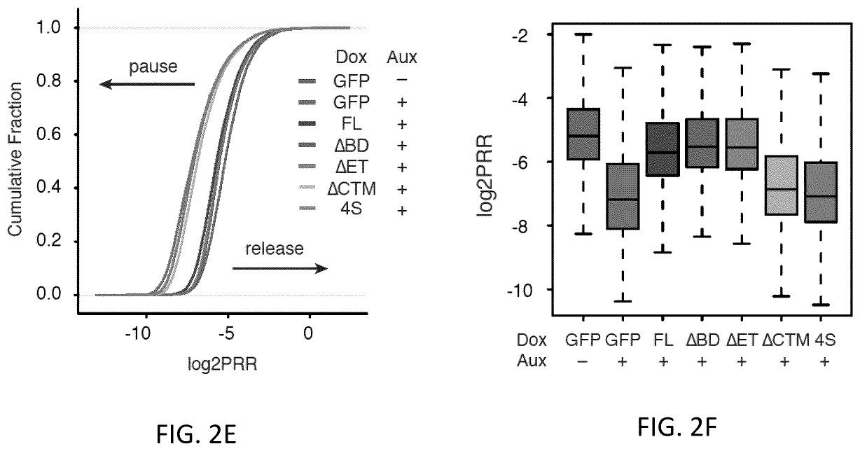

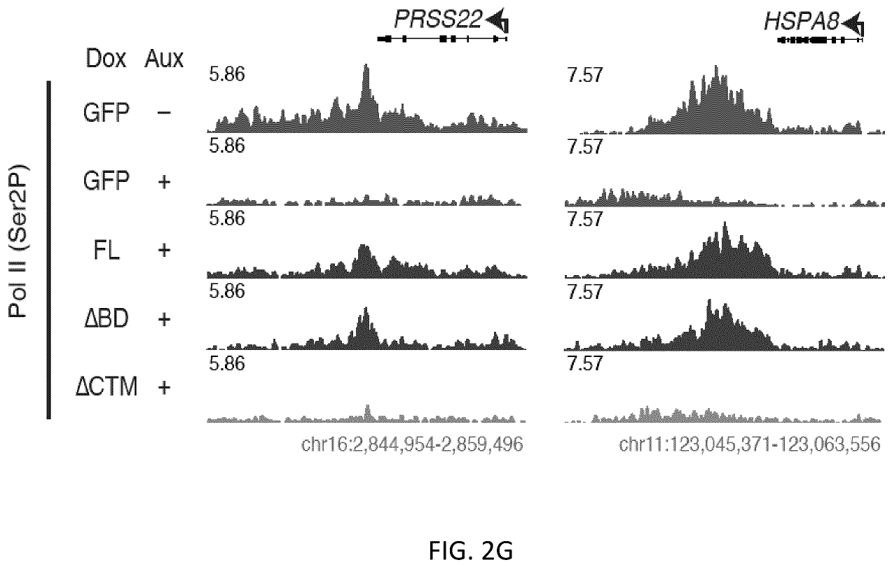

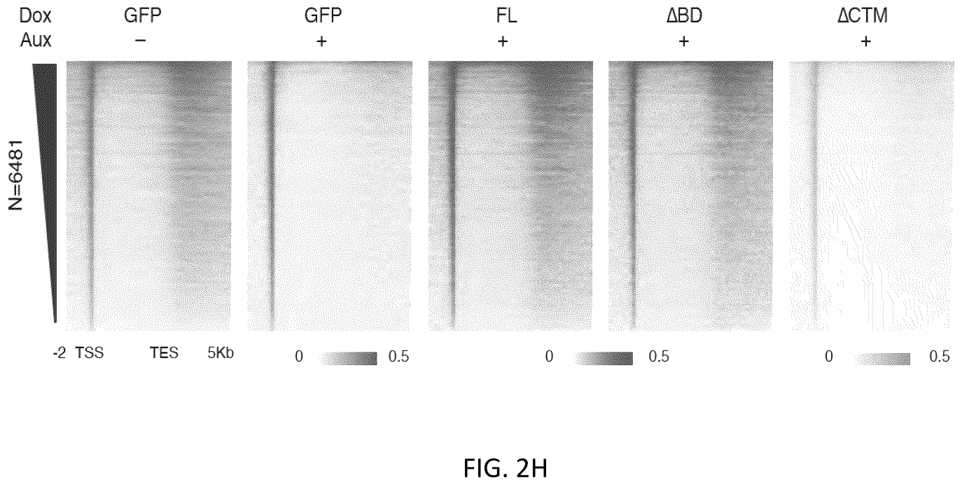

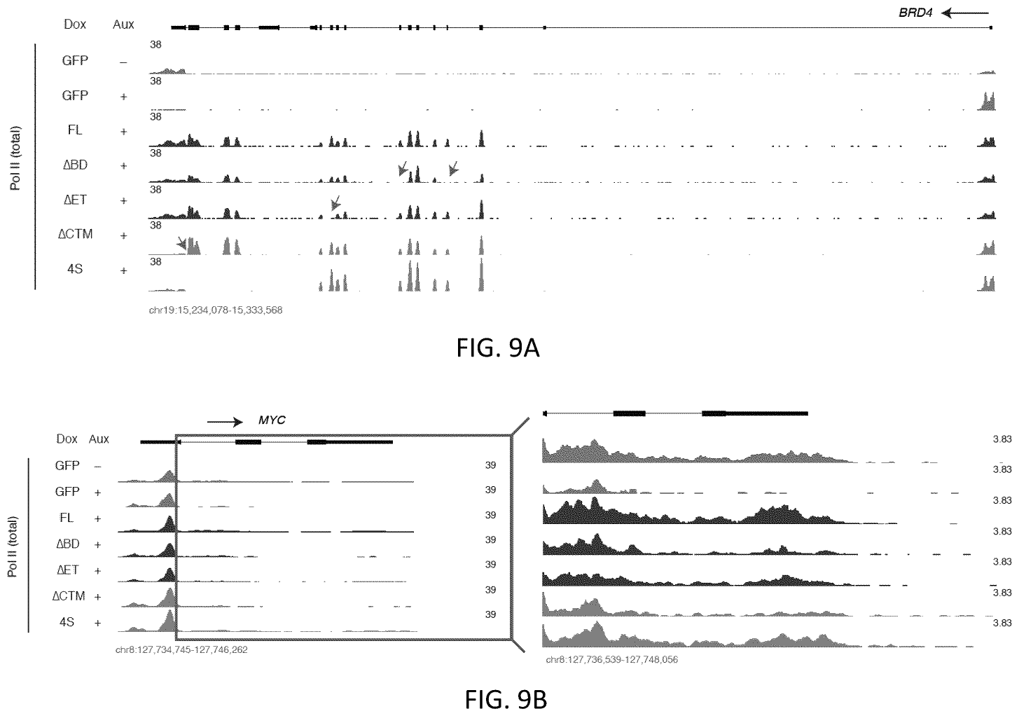

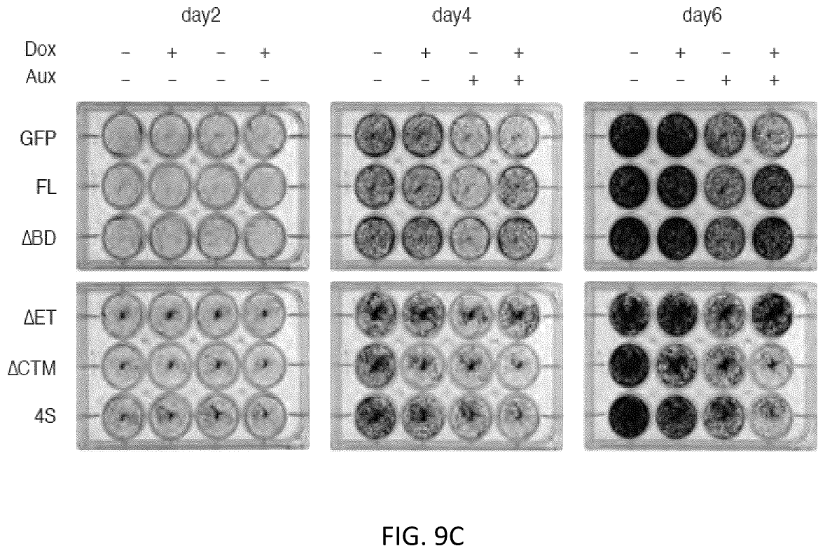

The finding that disruption of BRD4 binding to acetylated histone lysines was not sufficient to disrupt BRD4-mediated Pol II pause release (FIG. 1) naturally led us to hypothesize that PTEFb could release Pol II in a BRD4-dependent but bromodomain-independent manner. To address this hypothesis, we generated stable lines with Dox-inducible, FLAG-tagged BRD4 constructs, including full length (FL) BRD4 as well as bromodomains (ΔBD), extra terminal domain (ET) (ΔET) or C-terminal motif (CTM) (ΔCTM) domain deletion mutants, in BRD4-IAA7 cells. We also created a construct for a short, endogenously-expressed BRD4 isoform (BRD4S) that also lacks the CTM (FIG. 2A). GFP in the same backbone was used as a construct expression control. We first induced expression of the BRD4 constructs by Dox treatment for two days before rapidly depleting endogenous BRD4 with auxin for three hours and then carried out Pol II ChIP-seq (FIG. 2B). Surprisingly, we found that all three of the FL, ΔBD and ΔET constructs could rescue the Pol II pausing caused by depletion of endogenous BRD4, while the ΔCTM and BRD4S constructs could not (FIG. 2C). Genome-wide analysis of the Pol II ChIP-seq demonstrated a visibly inverted pattern of Pol II occupancy in cells expressing the ΔBD or ΔET but not ΔCTM constructs, indicating that the ability of BRD4 to release Pol II is affected by CTM deletion but not by bromodomains or ET domain deletion (FIG. 2D). As shown by boxplot and ECDF representations of log 2PRR, the ΔBD and ΔET mutant BRD4 constructs rescued Pol II release to an extent similar to that of WT (FL) BRD4, but ΔCTM and BRD4S did not (FIGS. 2E and 2D). Notably, upon addition of Dox, signal from Pol II that is actively transcribing genome-integrated BRD4 constructs is observable in the Pol II ChIP-seq, confirming the expression of our mutant constructs. (FIG. 9A). We also tested Pol II serine 2 phosphorylation (Ser2P), a modification at the Pol II CTD that is strongly associated with the elongation^{6,34} stage^{6,34}, by ChIP-seq for the FL, ΔBD and ΔCTM rescue conditions (FIGS. 2G and 2H). Consistent with the total Pol II ChIP-seq, FL and ΔBD constructs could rescue the loss of Ser2P signal caused by depletion of endogenous BRD4, while ΔCTM could not (FIGS. 2G and 2H). We did observe several genes (such as MYC) that were not fully rescued by the bromodomain-less BRD4, reflecting the pausing effect of JQ1 treatment at some genes (FIG. 9B). We also tested the ability of these mutants to rescue cell growth and confirmed that the bromodomain-less constructs can rescue the cell growth defect caused by endogenous BRD4 depletion (FIG. 9C).

### Bromodomain Dispensability is Conserved Across Cell Lines

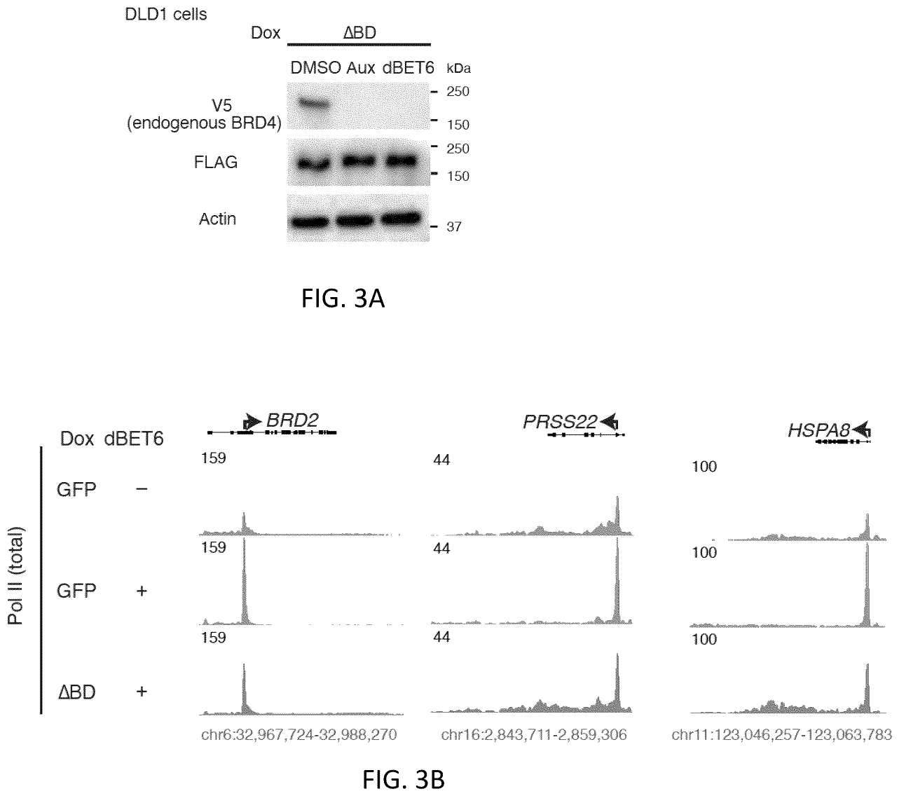

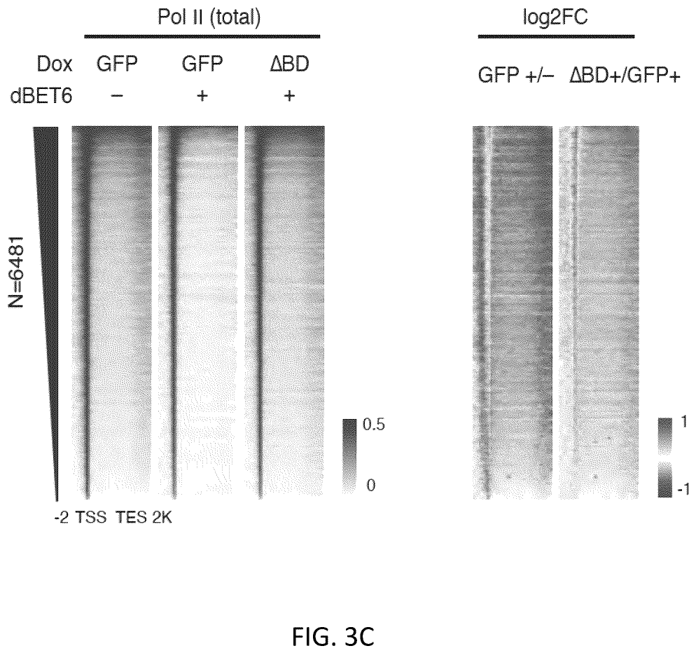

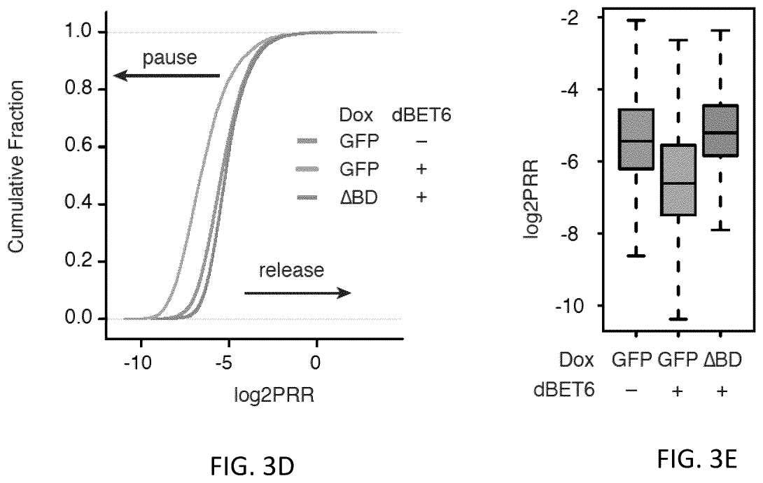

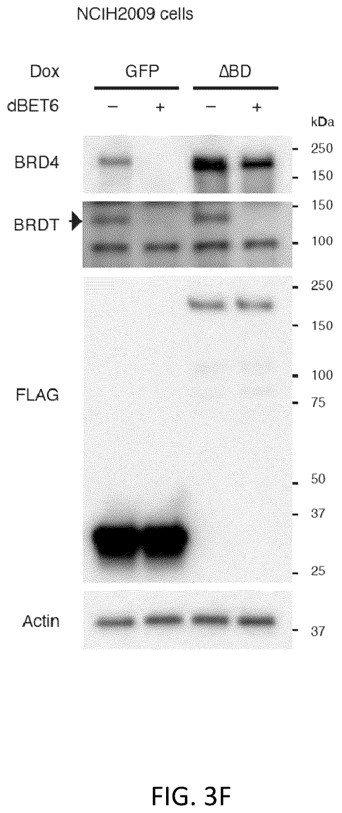

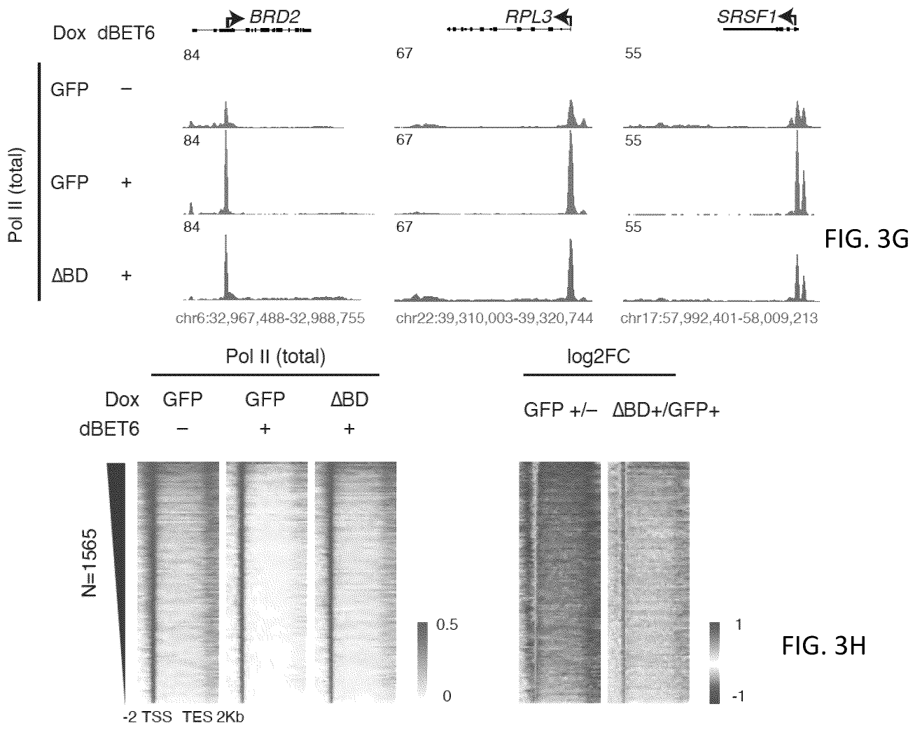

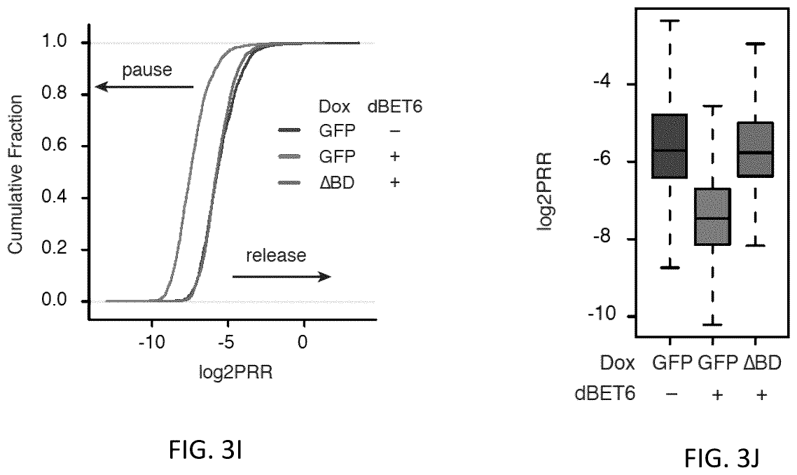

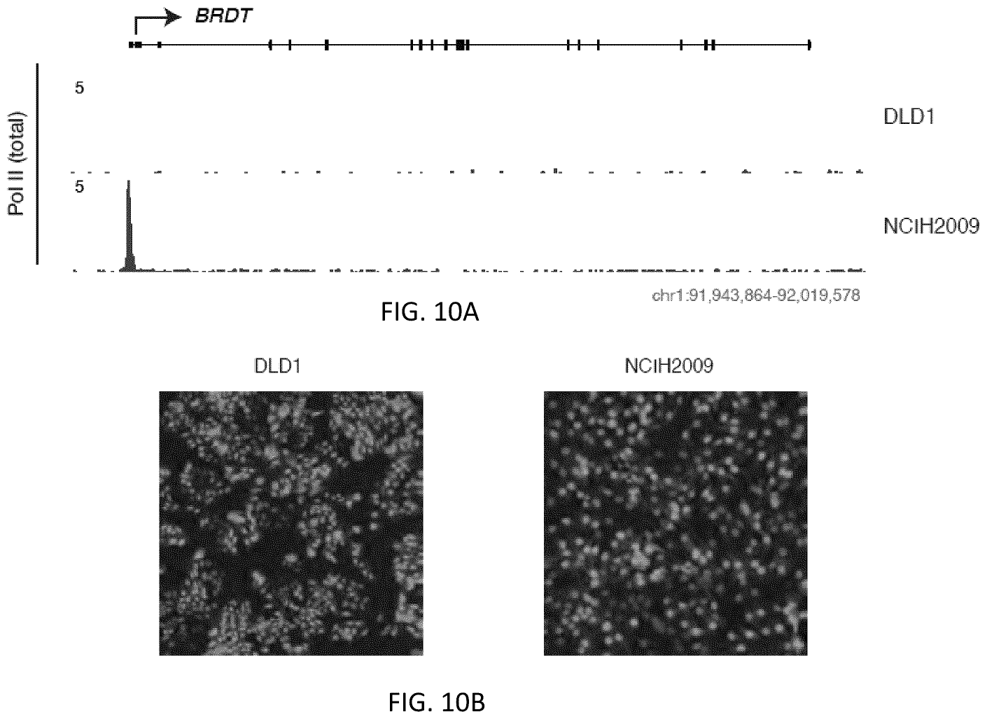

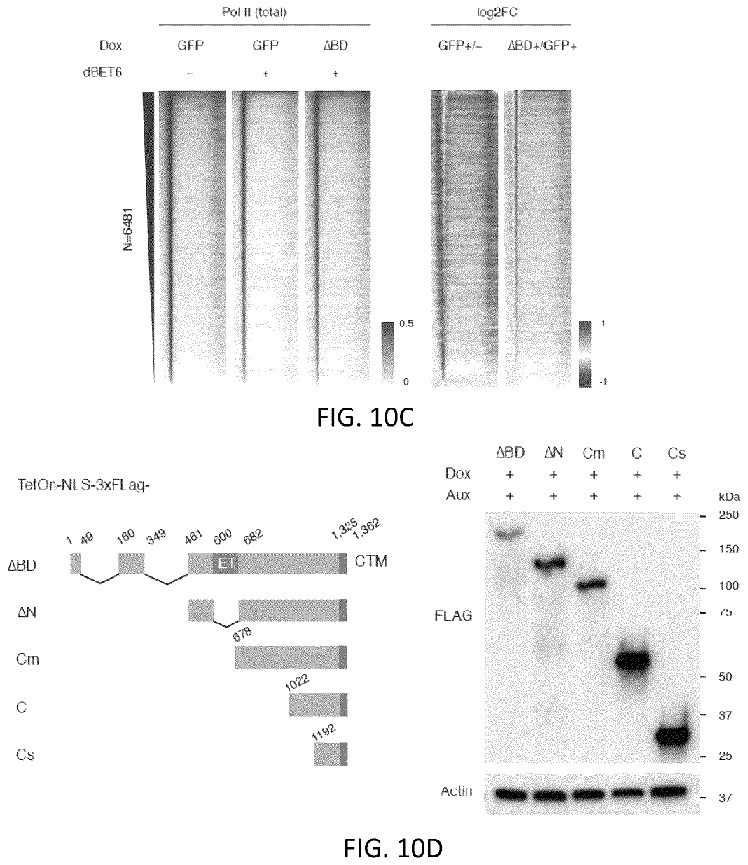

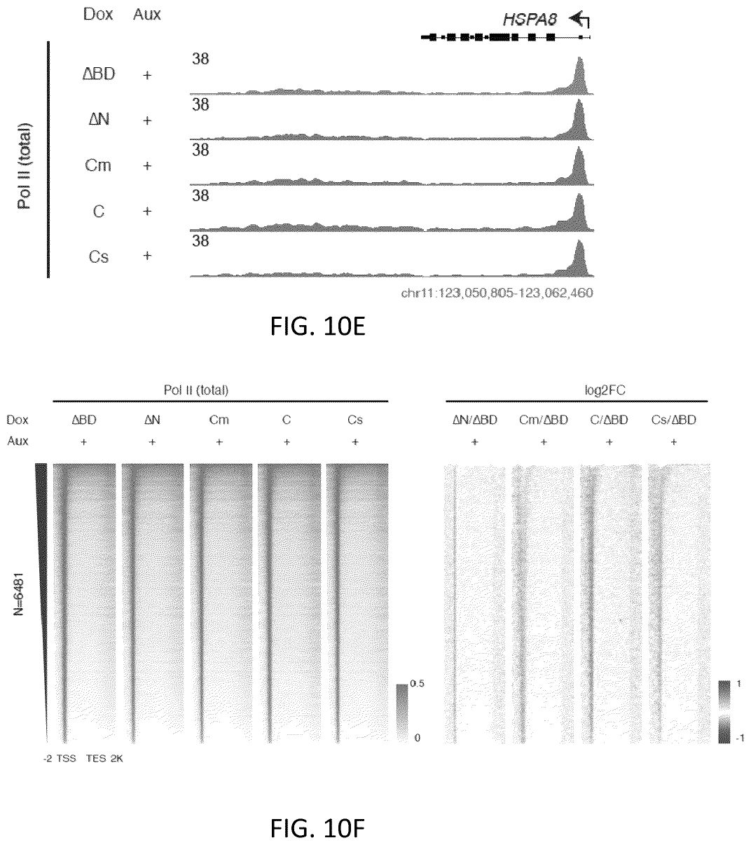

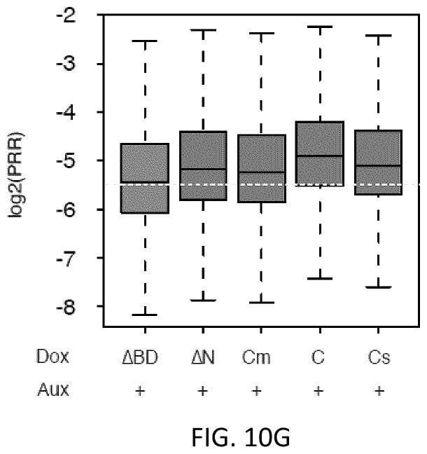

Since the bromodomain-less mutant BRD4 is resistant to dBET6 treatment (FIG. 3A), we treated cells expressing the bromodomain-less mutant with dBET6 to see whether the mutant could reverse the pausing effect normally seen upon dBET6 treatment. As expected, we found that dBET6-induced pausing is abolished by bromodomain-less BRD4 (FIGS. 3B-3E). We then tested whether this effect holds true beyond DLD1 cells by expressing the bromodomain-less mutant and testing the effect of dBET6 in a very different cell line, NCIH2009. In addition to BRD4, the NCIH2009 cells also endogenously express the BET family member BRDT, but both proteins are degraded by dBET6 (FIGS. 3F, 10A, and 10B). The genome-wide Pol II profile differs between DLD1 and NCIH20009, but we found that for most genes displaying dBET6-induced pausing in NCIH2009, this effect can be rescued by reconstitution with the bromodomain-less mutant (FIG. 3G). Genome-wide analysis in NCIH2009 further supported our conclusion that the bromodomain-less mutant rescues Pol II pause release in NCIH2009 cells (FIG. 10C). We isolated about a quarter of the genes displaying the highest degree of dBET6-induced pausing in NCIH2009 from the gene list, repeated the same analysis with these genes and found that most of the highly paused genes were rescued by the bromodomain-less BRD4 in NCIH2009 cells (FIGS. 3H-3J). Although the role of BRDT in transcription is largely unknown, it contains a PTEFb-interacting domain and could potentially function similarly to BRD4 in releasing paused Pol II, and bromodomain-less BRD4 may also rescue the effect of BRDT depletion in NCIH2009 cells. Nevertheless, our data indicates that the bromodomain-independent function of BRD4 in Pol II release is not strictly limited to DLD1 cells.

A Bromodomain-Less BRD4 C-Terminal Fragment Interacts with PTEFb and Rescues Pol II Pause Release

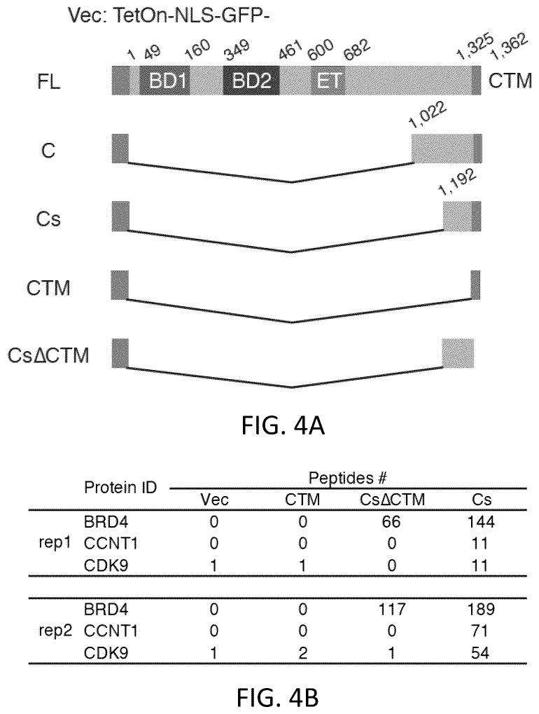

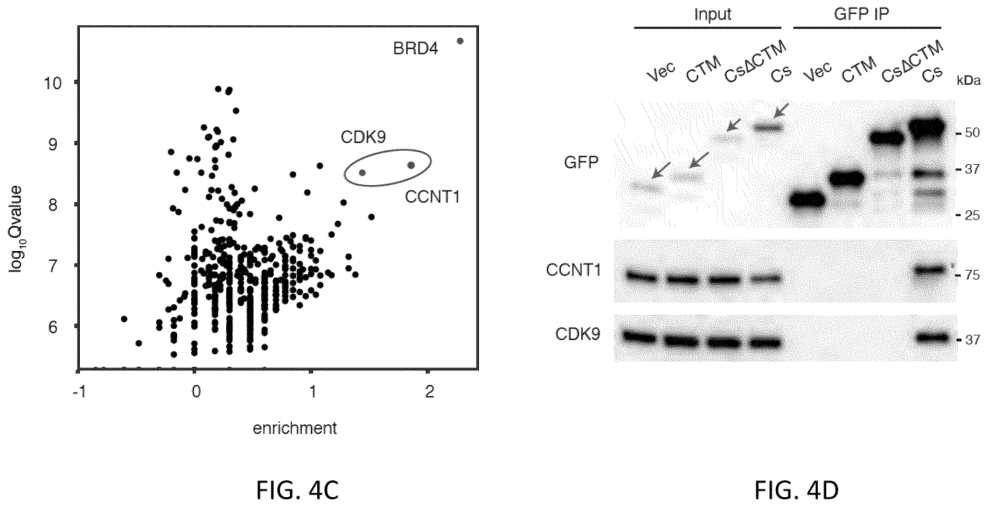

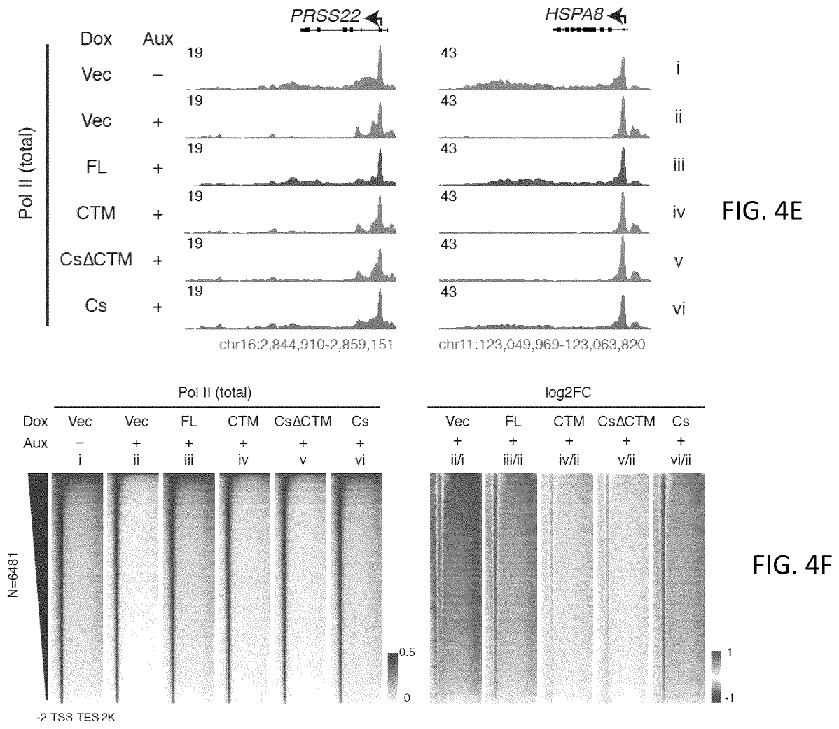

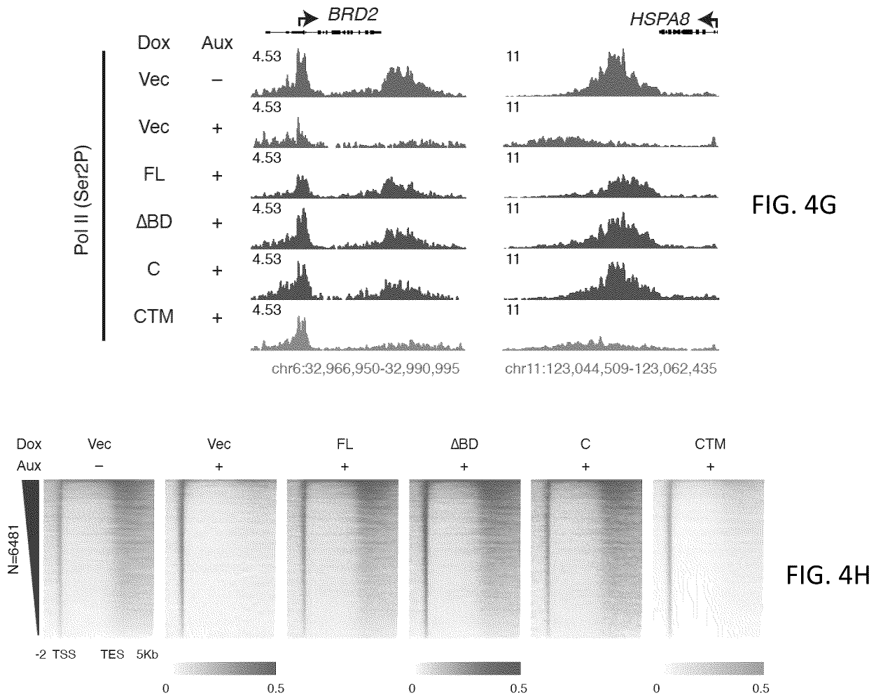

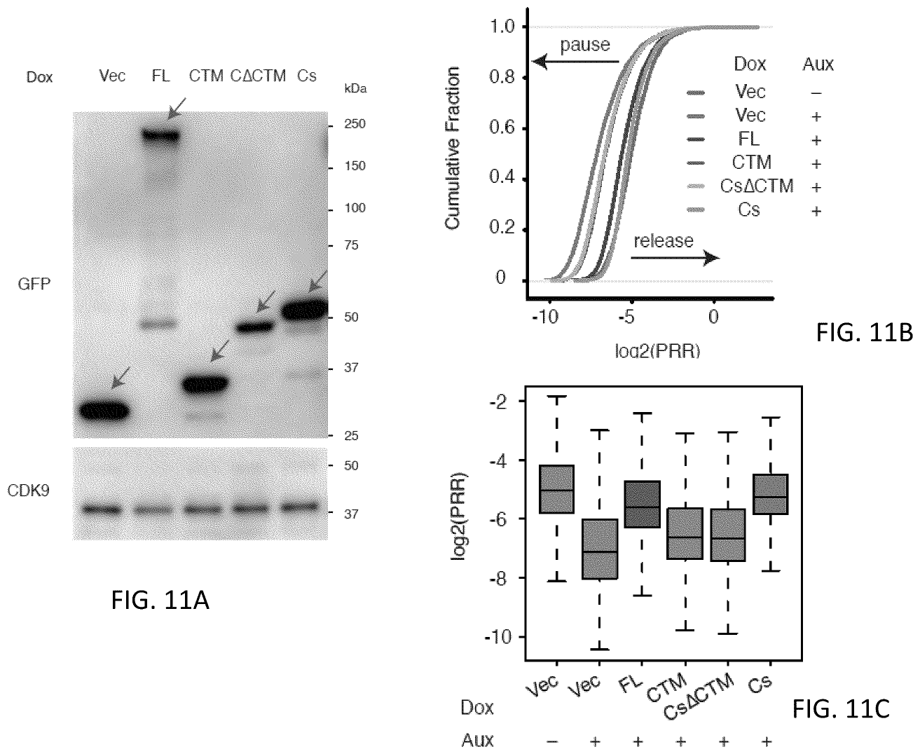

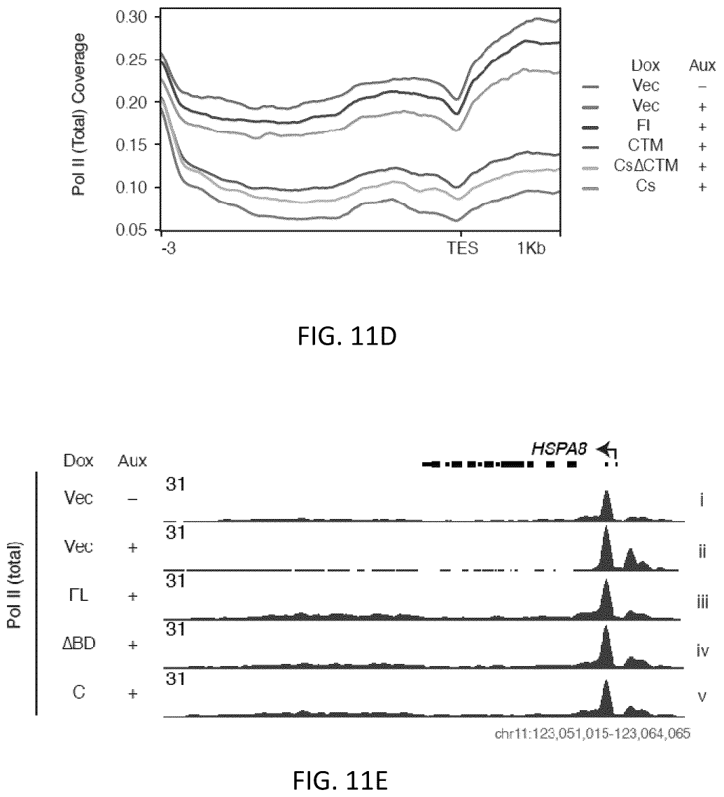

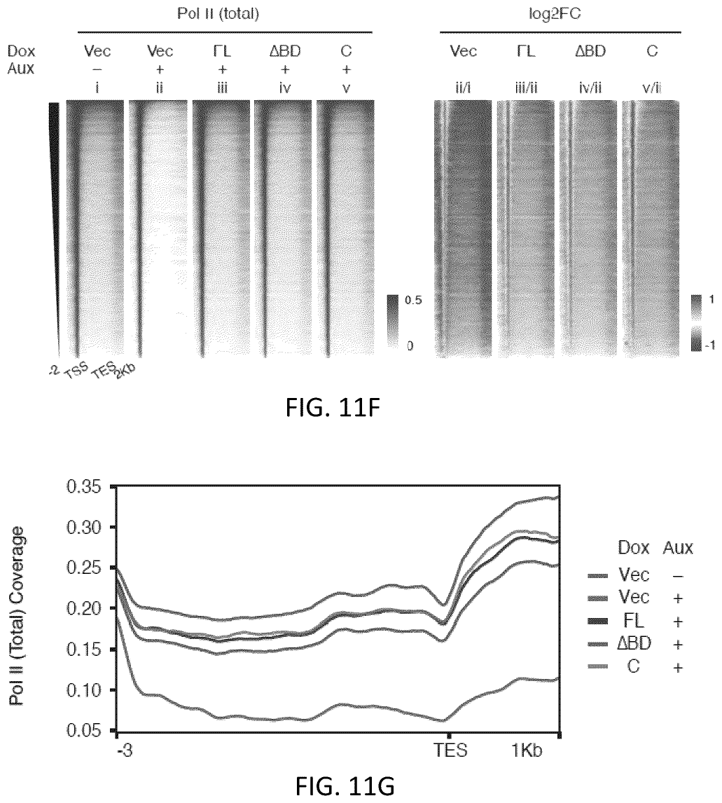

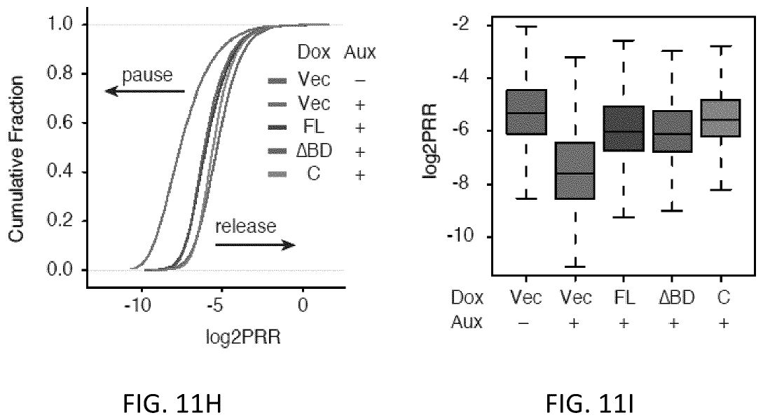

To investigate which BRD4 regions are required for the function of BRD4 in Pol II pause release, we generated a series of N-terminal truncation mutants retaining the CTM in the same FLAG-tagged vector as above (FIG. 10D). After validating the induced expression of these mutants (FIG. 10D), we used Pol II ChIP-seq to screen them using the same rescue experiment setup, with the bromodomain deletion mutant (ΔBD) as a negative control. To our surprise, the Pol II profile was similar for all the mutant constructs, with a noticeable decrease in gene body Pol II occupancy for only the shortest mutant construct (Cs), which consists of just 170 residues from the C terminus of BRD4 (FIGS. 10E and 10F). We calculated the log 2PRR and found that all the truncation mutants could rescue Pol II release to an extent equal or greater to that of the ΔBD construct, as indicated in the boxplot representation (FIG. 10G). The endogenous BRD4 acquired a 3xFLAG tag upon IAA7 tagging, so to exclude any potential confusion deriving from residual endogenous BRD4, we replaced the 3xFLAG tag with GFP for further characterization of the C-terminal constructs (FIG. 4A) (in our previous experience, FLAG ChIP-seq works poorly in DLD-1 cells, while GFP ChIP-seq works well.) We first validated the expression of the GFP-tagged shortest construct (Cs), Cs with CTM deletion (CsDCTM) and a CTM-only (CTM) construct by western blot (FIG. 11A). Then, we carried out immunoprecipitation followed by mass spectrometry (IP-MS) for these mutants. The consistent results of two replicate IP-MS experiments showed that only the Cs mutant retained the ability to interact with PTEFb (FIG. 4B). We assessed the Cs interactome, represented by interacting protein Qvalue vs their enrichment over that of the vector and found that two of the most outstanding Cs interacting proteins are components of the PTEFb complex, which we then validated by western blot analysis (FIGS. 4C and 4D). We further validated these results by carrying out Pol II ChIP-seq for the GFP-tagged mutant constructs in comparison with full-length (FL) BRD4. Consistently, the Pol II occupancy profile shows the rescue of Pol II pause release by the Cs construct (FIGS. 4E and 4F). Consistent with the FLAG-tagged version, despite noticeable reduction in gene body Pol II occupancy for the Cs mutant there was no corresponding defect in the Cs PRR relative to the FL BRD4 positive control (FIGS. 11B-11D). Since the PRR was actually highest for the extended C terminus (C) in our screen of FLAG-tagged mutant constructs (FIG. 10G), we also generated a GFP-tagged C construct (FIG. 4A) and compared its ability to rescue Pol II release to the FL and ΔBD constructs, finding no obvious defect in rescued Pol II occupancy at the gene body relative to the BRD4-FL positive control (FIGS. 11D-11I). We also confirmed the functionality of the C terminus by Ser2P ChIP-seq in the rescue experiment (FIGS. 4G and 4H). Multiple replicates of IP-MS, Pol II ChIP-seq, and Ser2P ChIP-seq experiments reproducibly consolidated our conclusion that the C terminus of BRD4 is sufficient for the release of paused Pol II.

### Distinct Layers of the BRD4-PTEFb Complex: Identification of a Histone Acetylation-Independent but Active BRD4-PTEFb Population

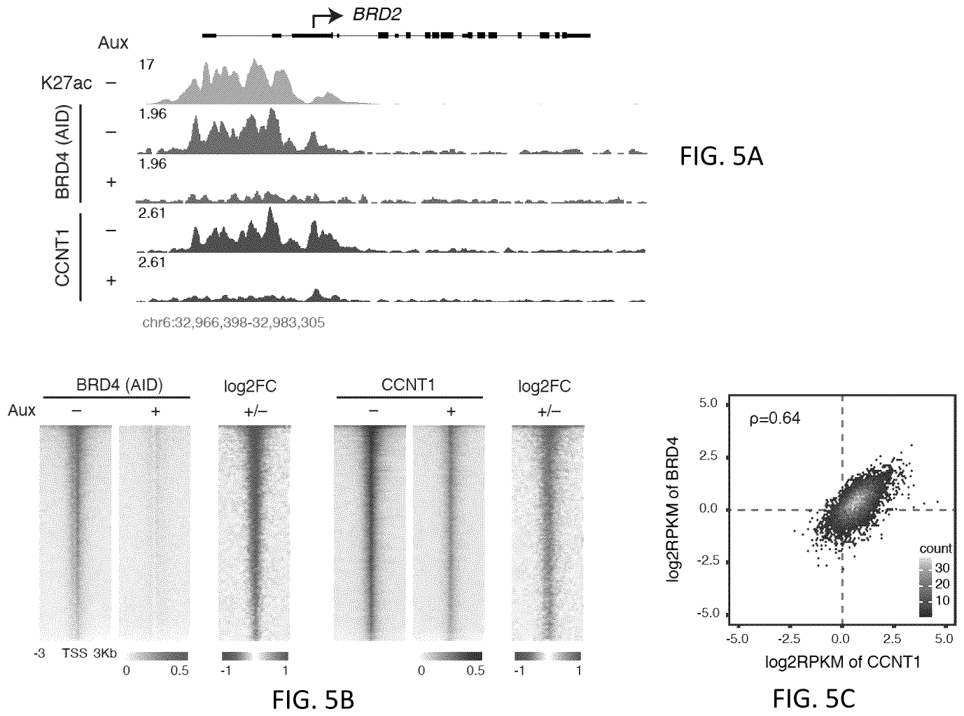

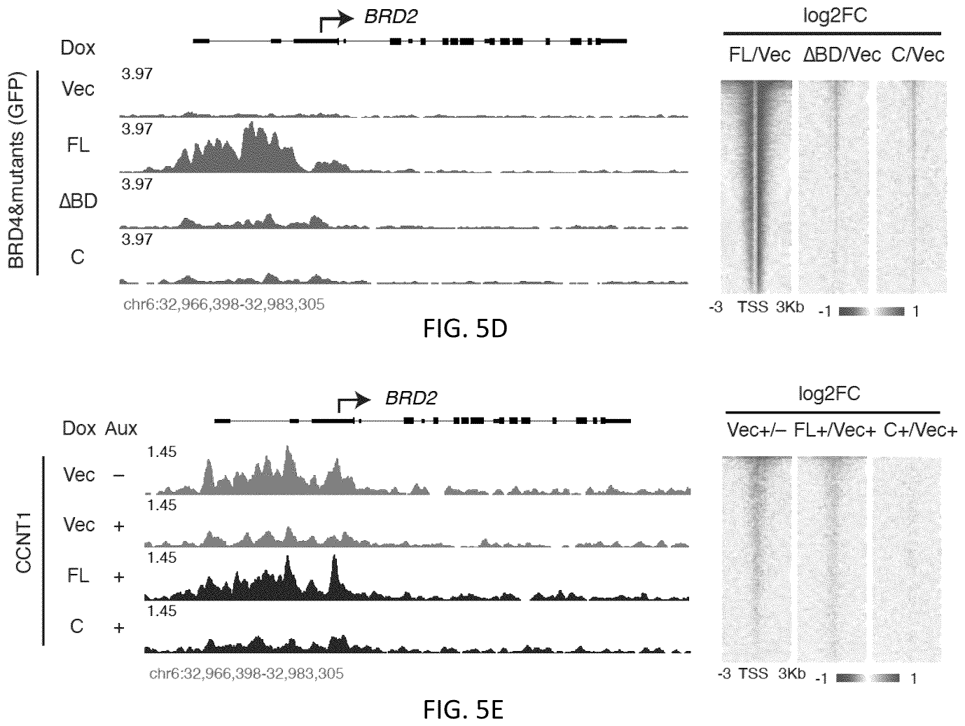

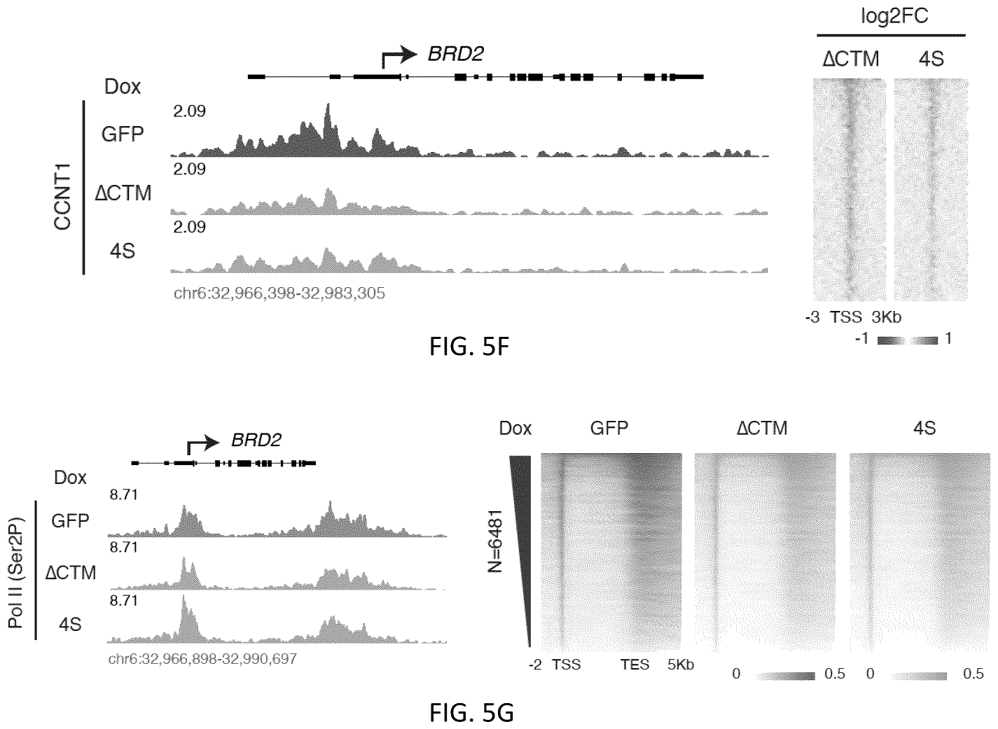

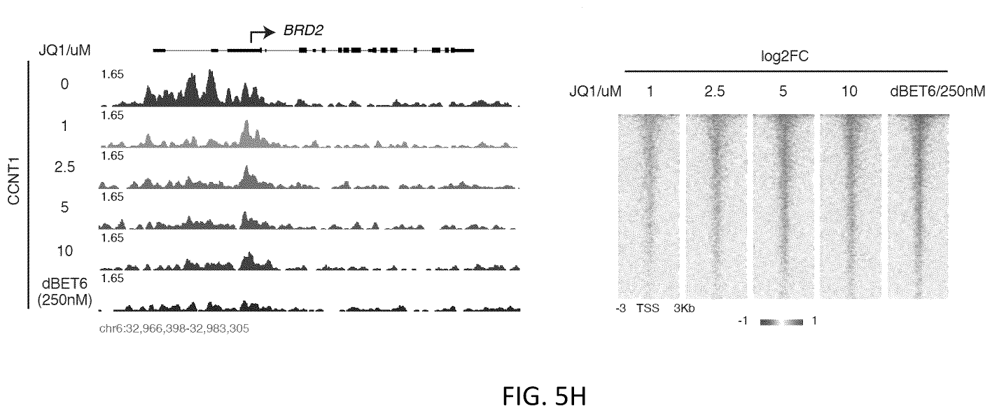

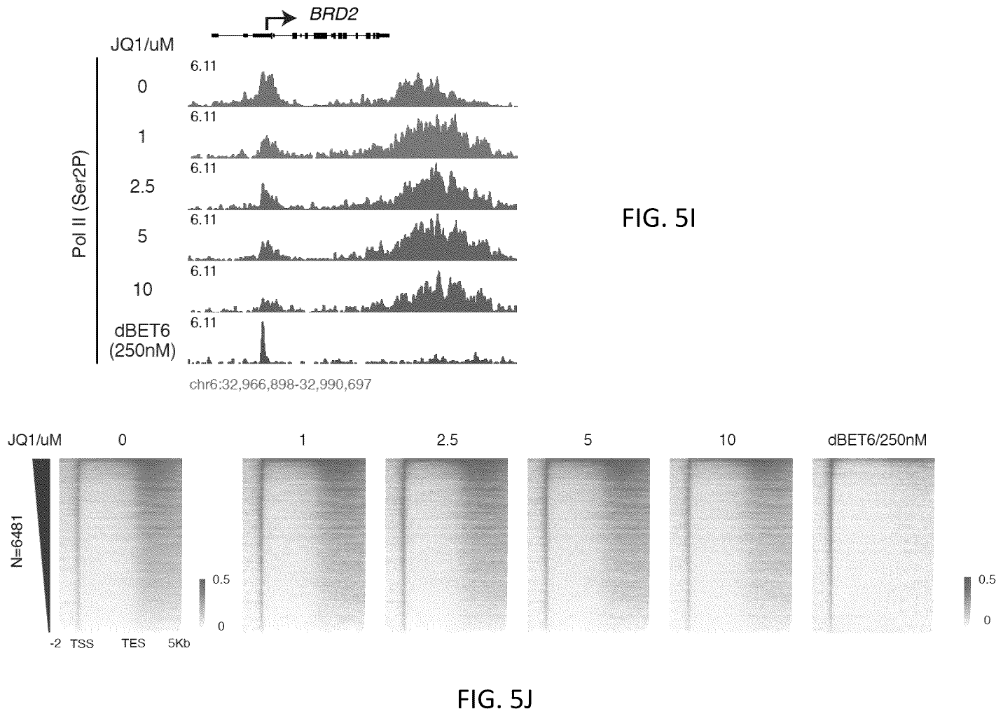

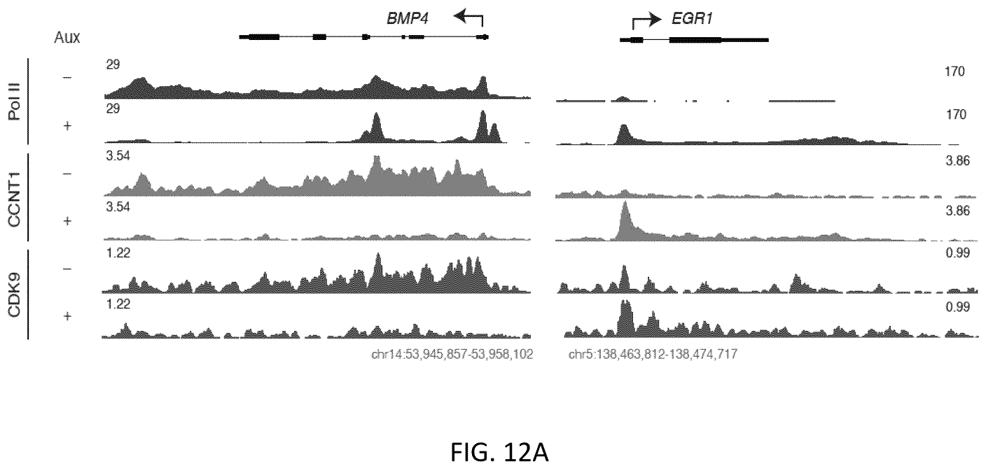

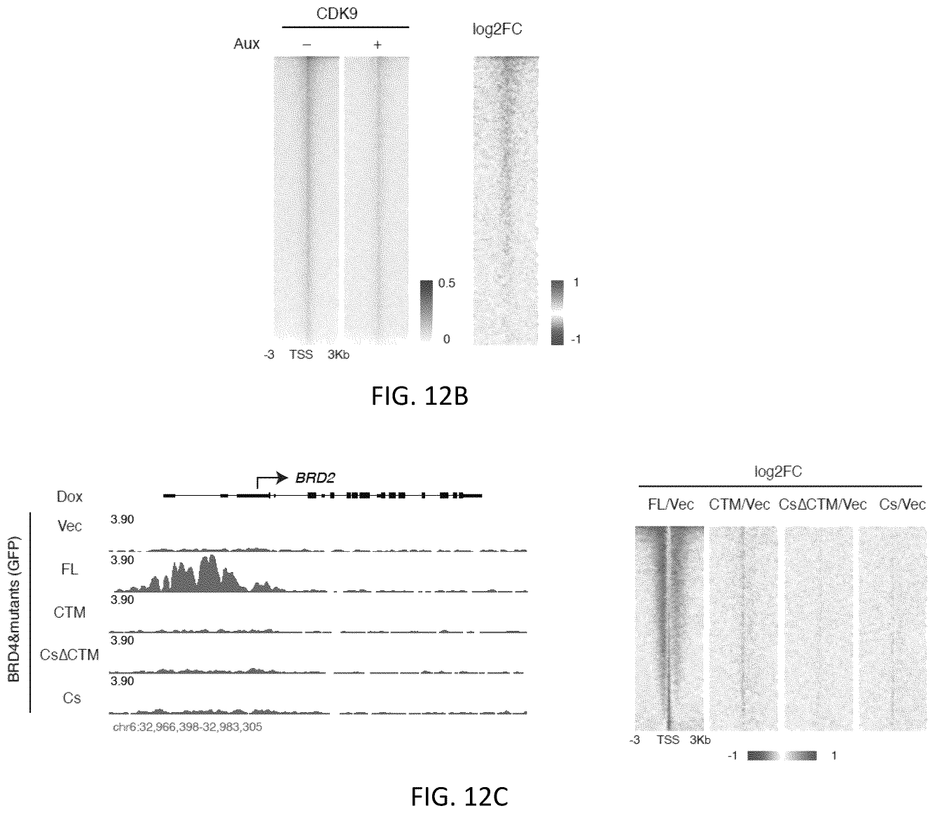

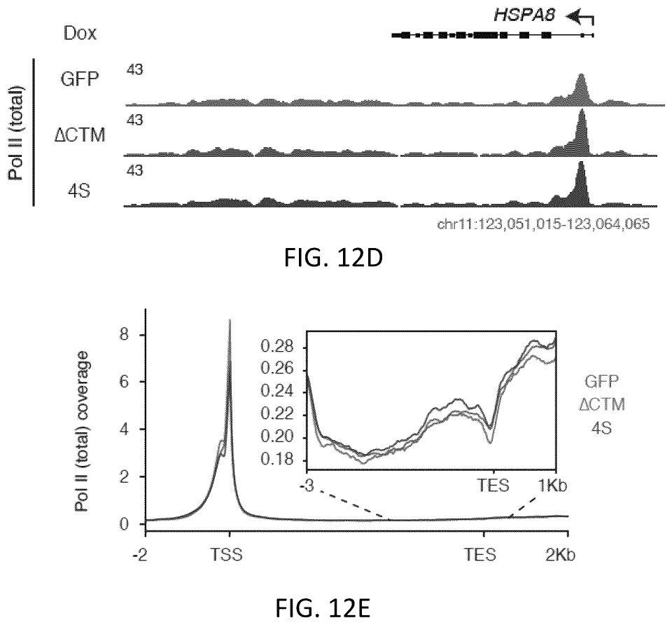

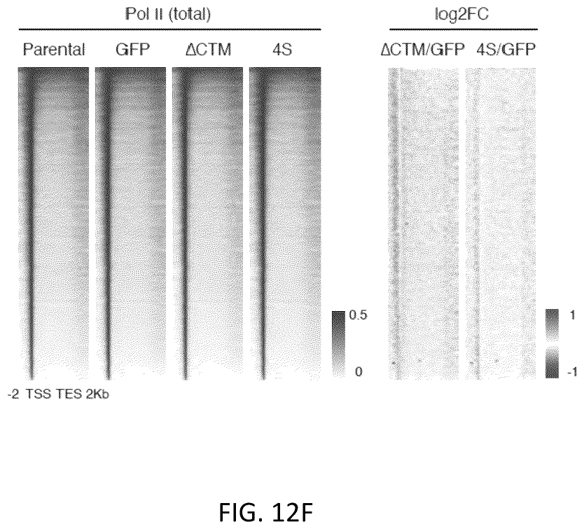

The extent to which BRD4 is required for recruiting PTEFb to chromatin and regulating gene expression remains a subject of some debate^{21,35,36}. Aiming to further investigate this point, we carried out ChIP-seq for Pol II, CCNT1 and CDK9 in BRD4-IAA7 cells with and without prior depletion of endogenous BRD4 by auxin treatment. BRD4 depletion led to a dramatic reduction in the CCNT1 peaks at promoters genome-wide, indicating that CCNT1 recruitment to chromatin relies on BRD4 (FIGS. 5A and 12A). We integrated this data with our previously published ChIP-seq in the BRD4-AID line and compared the peaks of CCNT1 and BRD4 to H3K27ac (FIG. 5A). Genome-wide analysis revealed general colocalization of CCNT1 and BRD4 at promoters, which was mostly associated with histone acetylation (FIGS. 5B and 5C). Signal was generally low in our CDK9 ChIP-seq, but at genes that were highly enriched with Pol II we were able to see CDK9 peaks with the same trend seen for CCNT1 (FIGS. 12A and 12B). Notably, a few genes show increased Pol II occupancy and thus retain PTEFb upon BRD4 depletion (FIG. 12A), which is presumably recruited by SEC (data not shown). Next, we carried out GFP ChIP-seq for the GFP-tagged BRD4-FL and mutants (FIG. 4A). Surprisingly, though they could all rescue Pol II occupancy and release, the ΔBD, C and Cs mutants had ChIP-seq signals that were minimal genome-wide compared to that of the FL BRD4 (FIGS. 5D and 12C). We then checked CCNT1 occupancy in the rescue experiments and found that CCNT1 signal was restored by BRD4-FL but not the bromodomain-less C mutant (FIG. 5E). This result is consistent with the GFP ChIP-seq results and further indicates that the population or “layer” of acetylation-bound BRD4-PTEFb captured by ChIP-seq is mainly associated with BRD4 bromodomains interacting with acetylation sites, while a distinct acetylation-independent layer, which is not captured by ChIP-seq, nevertheless functions to release Pol II (FIG. 5E). To further confirm that disruption of the acetylation-bound BRD4-PETFb layer alone could not abolish Pol II pause release, we introduced an acetylation-bound but phosphorylation-defective BRD4-PTEFb layer by overexpressing the CTM-less (and therefore PTEFb-blind) BRD4 mutants to displace the population of acetylation-bound BRD4-PTEFb. Indeed, a large portion of BRD4-PTEFb was knocked off from chromatin by overexpression of either the DCTM or 4S CTM-less BRD4 mutants (FIG. 5F). However, this displacement did not constitute a dominant negative effect, as the ChIP-seq signals for both total Pol II and Ser2P were not noticeably diminished by CTM-less construct overexpression (FIGS. 12D-12F). To further validate this observation with a more severe disruption of the acetylation-bound layer, we then treated cells with increasing concentrations of JQ1. We also included dBET6 treatment as a control for joint disruption of both the acetylation-bound and acetylation-independent layers. Though CCNT1 ChIP-seq signals decreased dramatically with increasing concentrations of JQ1, concomitant change in the Ser2P ChIP-seq signals was minimal, and only with dBET6 disruption of both layers did Ser2P signal fade away (FIGS. 5H-J). These results suggest that disruption of the acetylation-independent BRD4-PTEFb layer is the underlying mechanism by which dBET6 differs from JQ1 in effectively abolishing Pol II pause release.

### The BRD4 C-Terminal Domain is Sufficient for CyclinT1 Binding in BRD4-PTEFb Complex

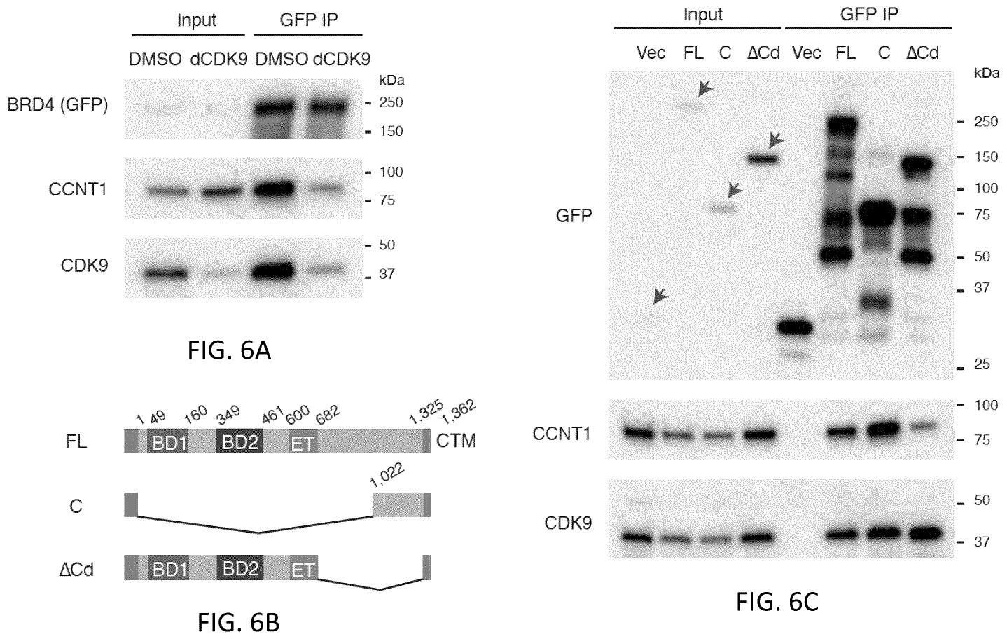

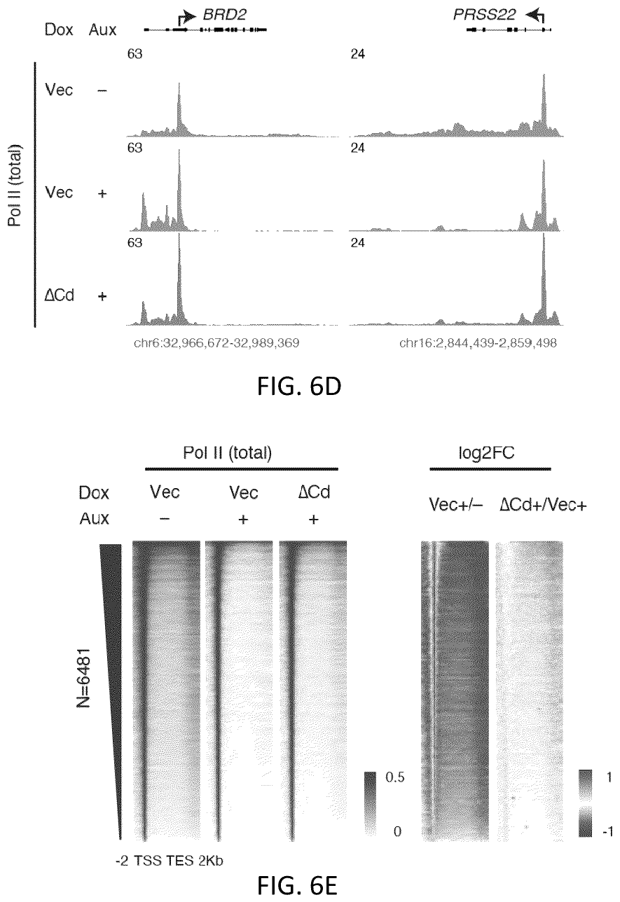

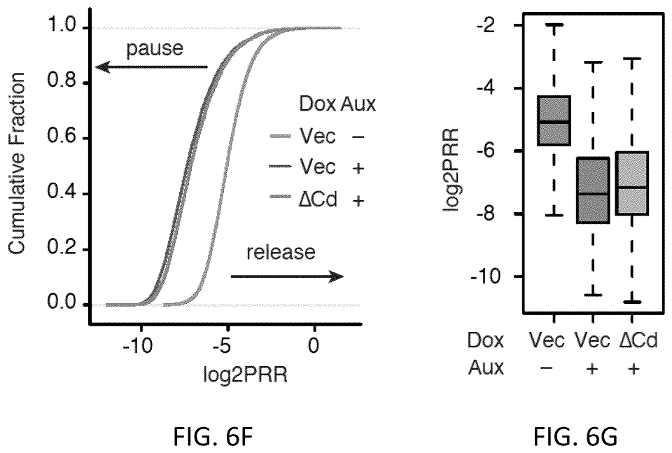

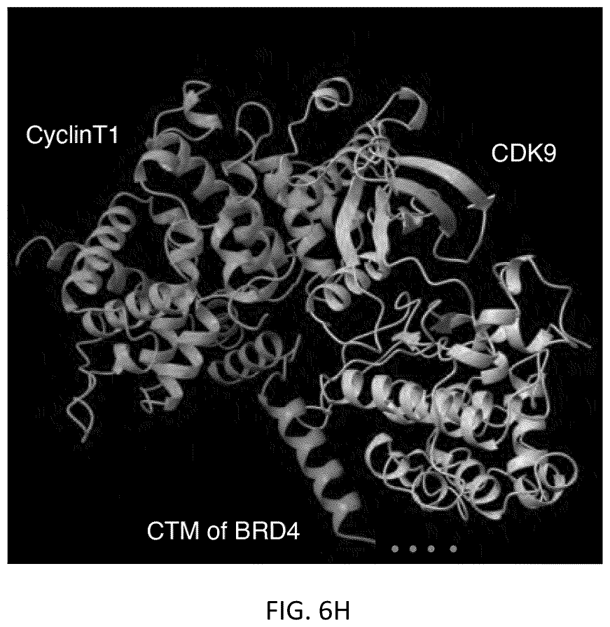

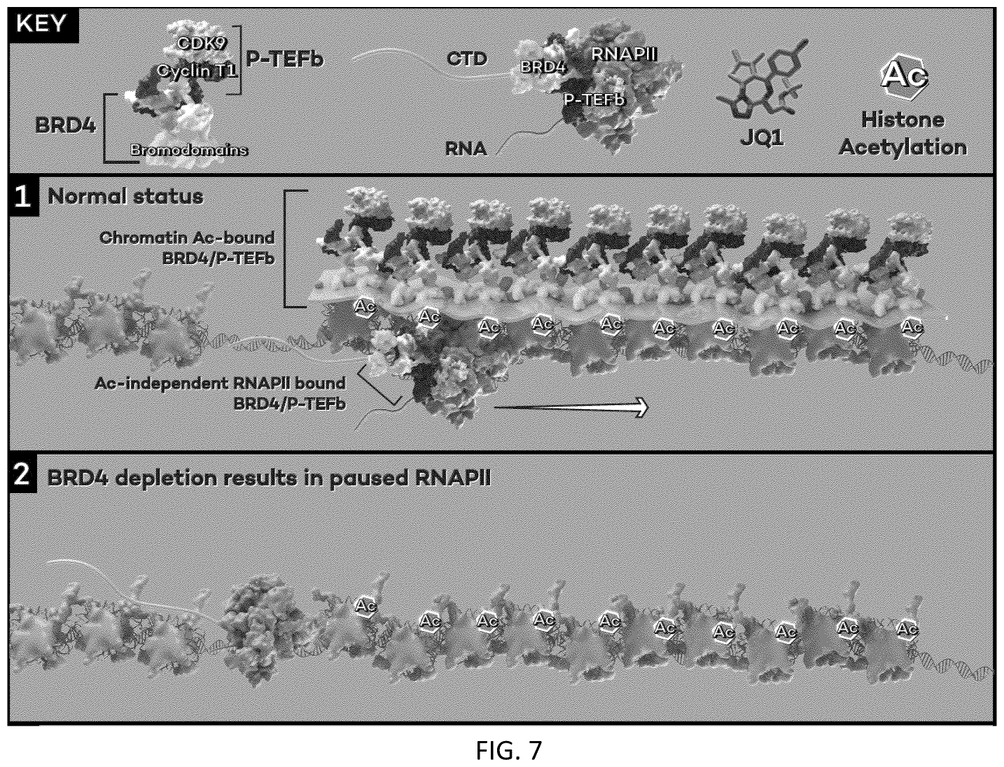

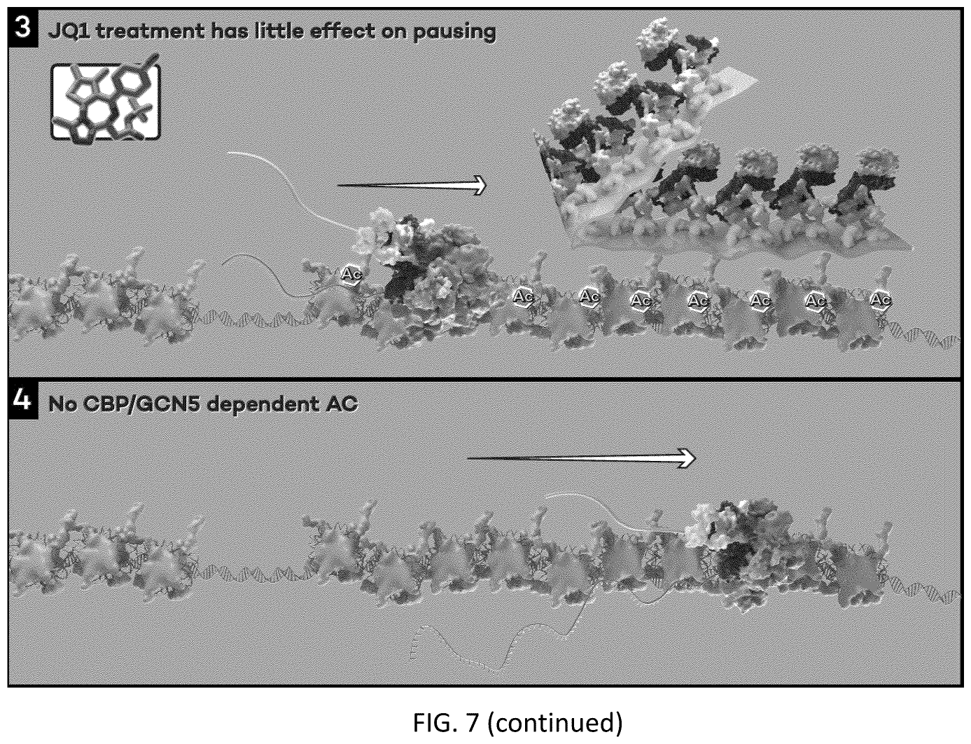

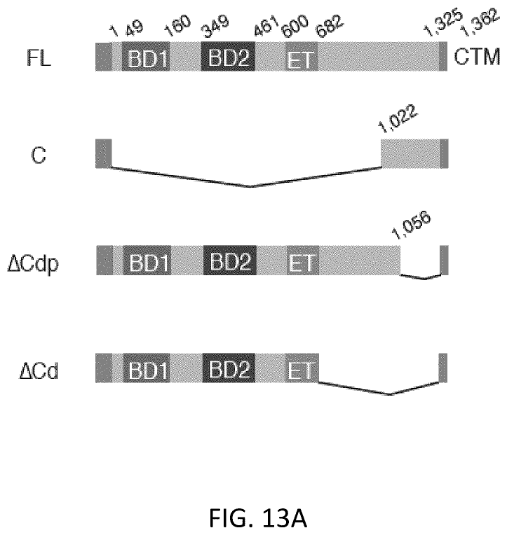

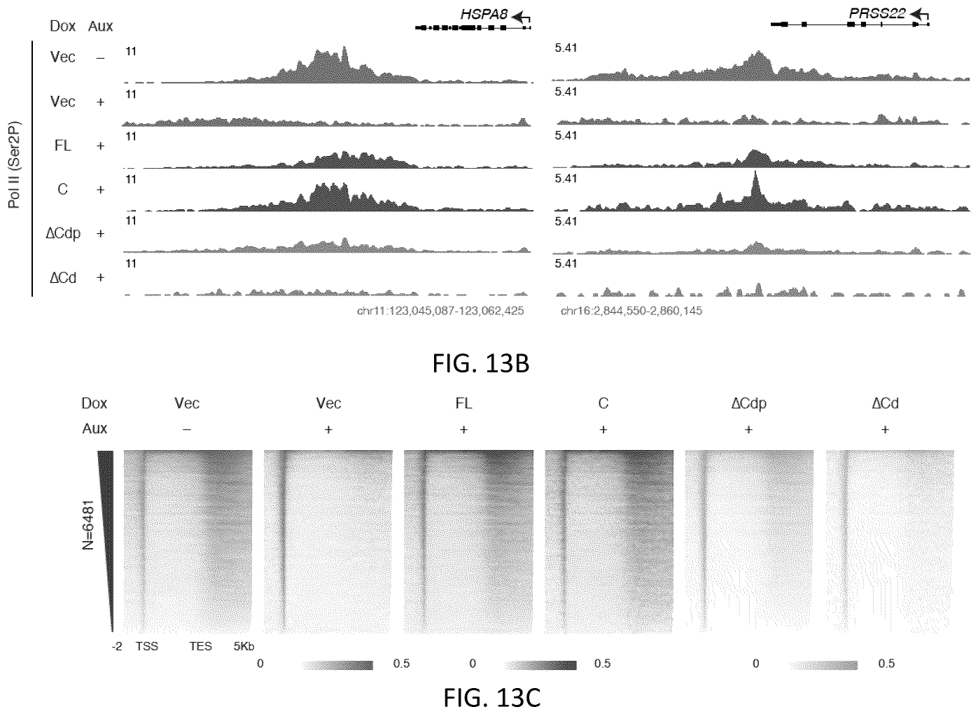

The fact that the GFP-tagged CTM cannot pull down PTEFb, while the extended Cs can, prompted us to investigate how the C terminus of BRD4 mediates its interaction with PTEFb. We first carried out GFP IP for the GFP-tagged, full-length BRD4 upon dCDK9 treatment. CDK9 degradation also impairs BRD4-CCNT1 interaction, indicating the requirement of CDK9 for stable BRD4-PTEFb complex formation (FIG. 6A). We then generated GFP-tagged partial (ΔCp) and entire (ΔCd) C-terminus deletion mutants (FIGS. 6B and 13A). Ser2P ChIP-seq in the rescue experiment revealed that though Pol II-releasing activity was largely impaired in the partial deletion mutant, the entire deletion almost completely abolished activity (FIGS. 13B and 13C). We carried out GFP IP for the ΔCd mutant and found a severe loss of binding to CCNT1 but not CDK9 compared to the FL and C mutants (FIG. 6C), which could explain why the ΔCd mutant cannot rescue Pol II release, as was further confirmed by the total Pol II ChIP-seq (FIGS. 6D-6G). Finally, we computed the predicted structure of the CTM of BRD4 C (37 residues) in association with the PTEFb complex (initial N-terminal 344 residues of CDK9 and the initial 293 N-terminal residues of CCNT1, as the corresponding crystal structure has been published³⁷) using AlphaFold integrated in ChimeraX³⁸. In line with the results of this study, the predicted structure highlights a critical role for the CTM in mediating the interaction between BRD4 and PTEFb, with CTM protruding into the pocket created by CDK9 and CCNT1 (FIG. 6H).

### DISCUSSION

In this study, we sought to determine why broad genome-wide changes in Pol II occupancy are seen upon BRD4 depletion but not bromodomain inhibition. Our depletion and rescue strategy allowed us to directly compare exogenous wildtype BRD4 with mutant BRD4 constructs, including bromodomain-less mutant BRD4 constructs that are unable to bind acetylated histone lysine residues. By characterizing the effects of these constructs on the Pol II profiles and its elongating form of Ser2P, we determined that while the C terminus of BRD4 is required for its participation in Pol II pause release, BRD4 bromodomains are dispensable for this function. We further validated the essential role for the BRD4 C terminus, isolating a minimal C-terminal fragment that lacks bromodomains but physically interacts with PTEFb and is sufficient to replicate the role of full-length BRD4 in Pol II pause release in cells. Displacing endogenous BRD4 from chromatin by overexpressing a PTEFb-blind mutant reduced the PTEFb chromatin occupancy without affecting Pol II pause release, phenocopying the effect of bromodomain inhibitor treatment. Importantly, this result indicates that endogenous bromodomain-containing BRD4 can release paused Pol II irrespective of its histone acetylation association. The endogenous expression of the BRD4 short isoform (4S), which lacks the CTM required for PTEFb interaction and thus cannot participate in release of paused Pol II, suggests that this displacement is physiologically relevant.

Our results all support a previously unappreciated role for BRD4 in acetyl recognition-independent release of paused Pol II. In the model emerging from our results, there are two distinct populations or “layers” of BRD4-PTEFb: a first layer of acetylation-bound BRD4-PTEFb complemented by a second layer of acetylation-independent BRD4-PTEFb that effectively releases paused Pol II. One question emerging from this model is what the role of the massive acetylation-bound BRD4-PTEFb layer is in transcription. Further studies are needed to dissect the endogenous functions of full-length BRD4 and its short isoform in potential balancing the two layers of BRD4-PTEFb. Nevertheless, this new angle on BRD4 function brings clarity to the long-running debate concerning the cause of insensitivity or resistance to BET inhibitors that have previously been observed in multiple studies and trials³⁹⁻⁴¹. Notably, other studies have also shown that BRD4 is required for survival even in BET inhibitor-resistant cells, similarly implicating a function for BRD4 beyond that mediated by its bromodomains⁴²⁻⁴⁴. Our findings represent a major step towards mechanistic understanding of this function, as they reveal the crucial importance of BRD4's C terminus for CDK9/CCNT1 interaction and regulation of Pol II pause release. Future studies would be focused on determining the structural basis of the interaction of BRD4 with CDK9 and CCNT1 and developing BRD4 C-terminal specific inhibitors, which (unlike pan-BET inhibitors ⁴⁵) would leave the acetylation-bound layer of BRD4-PTEFb and other BET family proteins unaffected. We predict that this class of inhibitors will be highly efficient in abolishing the function of BRD4 in Pol II pause release, resulting in potent therapeutic benefit.

In support of a bromodomain-independent function for BRD4, Sankar et al. recently mutated all 28 H3 alleles from K27 to R27 to show that H3K27 modification is not required for proper loading of the Pol II machinery onto chromatin or for transcriptional activation during cell fate transition in mESCs²⁶. Similarly, Zhang et al. found that abolishing H3K27ac was not sufficient to disrupt enhancer activity²⁷. Our finding, that BRD4-dependent transcription has no requirement for interaction with histone acetylation, may offer an explanation. Thus, H3K27ac may be better considered as an active transcription marker that reflects gene expression status rather than as a determinant for transcription or enhancer activity. The advantage for the cell to dispense with any histone modification requirement is that during conditions such as cell division and differentiation, rapid and specific gene expression changes could proceed undelayed by the process of histone modification establishment. Similarly, when cells respond to stress conditions such as heat shock, transcription is rapidly up- and down-regulated without respect to histone marks⁴⁶.

We did find a handful of genes that are upregulated upon depletion of endogenous BRD4 and for which BRD4 or mutant construct rescue had very different (or opposite) effects from those seen at the vast majority of genes. Because these genes were so few, they were not excluded from and did not affect our genome-wide analyses, but are certainly worth considering separately. Future studies will be needed to address the mechanism of transcriptional activation for these genes. We also observed several genes that were not fully rescued by the bromodomain-less BRD4 mutants, including the gene encoding the important and well-characterized transcription factor Myc. Since the MYC loci is known to be regulated by a super enhancer, and because super enhancers are bound by Mediator and BRD4 through bromodomains³¹, we suspect that the defect in MYC rescue could reflect a requirement for the histone-bound layer of BRD4-PTEFb in the maintenance of super enhancer activity. BRD2 has also been shown by our lab and others to be essential for transcriptional regulation⁴⁷⁻⁴⁹.

### Materials and Methods

### Cell Culture

DLD1 human cells were purchased from ATCC (CCL-221) and cultured in DMEM (Corning, #10013CV) supplemented with 10% FBS (Sigma-Aldrich, #F2442), 1% Glutmax (Gibco, #35050061), and 1% PS (Gibco, #15140122), in 37° C. incubator with 5% CO2. For drug treatment, auxin (#ab146403) was purchased from abcam. Doxycycline (#72742) was obtained from Stem Cell Technologies. JQ1 (#4499) was purchased from Tocris. dBET6 (#S876202) was purchased from Selleckchem. NVP-2 (#HY-12214A) and dCDK9 (#HY-123937) were purchased from MedChemExpress.

### Generation of Degron Cell Lines

BRD4-IAA7 DLD1 degron cells were generated similarly to previously described13. Specifically, PX330 (Addgene, #42230) altered with the insertion of sgRNA (#1 AATCTTTTCTGAGCGCACCT (SEQ ID NO.: 6), or #2 ATCAAAGTCAGAAGCCACCT (SEQ ID NO.: 7)) targeting the stop codon area of the BRD4 genomic locus was co-transfected with a donor plasmid using the Lipofectamine 3000 Transfection Reagent (Invitrogen, #L3000001) to trigger donor integration at the target site via homologous recombination repair. The donor plasmid, which we designed to integrate the IAA7 tag after the final BRD4 exon and introduce the F-box protein AtAFB2 under the EFIa promoter, was made using the pBlueScript II SK (+) backbone and synthesized gBlocks for the AA7-AtAFB2 pair and antibiotic selection marker. Upon transfection, DLD1 cells were selected for colony formation in the presence of either Geneticin (Gibco, #10131027) or hygromycin B (Invitrogen, #10687010) for 2 weeks. Single clone colonies were picked and verified by PCR and western blot analysis.

### Generation and Expression of BRD4 Mutant Constructs

TetOn lentiviral vector (#110280), BRD4-FL cDNA (#31351), and BRD4-ΔET cDNA (#21938) were obtained from Addgene. The BRD4S mutant was generated from TetOn-FLAG-BRD4-FL using the Q5 Site-Directed Mutagenesis Kit (NEB, #E0554S). All other BRD4 mutant constructs were generated via NEBuilder HiFi DNA assembly reaction (NEB, #E2621S) with synthesized gBlocks (IDT and Twist Biosciences). TetOn-BRD4 lentiviral constructs were amplified by transformation of stable competent E. coli (NEB, #C3040H). All BRD4 plasmid insertions were verified by Sanger sequencing (ACGT). Lentivirus for transducing mammalian expression of the BRD4 mutants was generated by co-transfecting BRD4 mutant expressing plasmids with pspax2/pmd2.g lentiviral packaging plasmids in 293T cells in 6-well format. Lentivirus was collected and filtered for infection of BRD4-IAA7 DLD1 cells in 6-well plates. Infected cells were selected with Blasticidin (Gibco, #A1113903) for two weeks. BRD4 mutant expression was achieved by adding 50 nM Dox into the medium and incubating for 48 h.

### Cell Growth Assay

million FLAG-tagged mutants transfected and Blasticidin selected BRD4-IAA7 cells were seeded in 12-well plates on Day 0. Dox was added to induce the mutants' expression. On Day 2, changed to fresh medium and added Dox to maintain the mutants' expression while adding auxin to deplete the endogenous BRD4. On day 4, changed to fresh medium and maintained with Dox and auxin. On Day 2, 4 and 6, when collected, each duplicated plate was fixed with 4% paraformaldehyde solution (Fisher Scientific, #50-980-495) in PBS for 20 min at room temperature under shaking. Then plates were washed with tap water for 3 times and dried overnight. Violet solution (Millipore Sigma, #HT90132) was used to stain the fixed cell.

### Western Blot

Whole cell lysates for western blot were prepared by directly lysing the cells with Laemmli sample buffer (Bio-Rad, #1610747) and boiling for 10 minutes. BRD4 (#13440), β-Actin (#3700), V5-Tag (#13202), HA-Tag (#3724S), CDK9 (#2316), and Cyclin TI (#81464) antibodies were purchased from Cell Signaling Technology. Tubulin (#E7) antibody was obtained from Developmental Studies Hybridoma Bank (DSHB). FLAG antibody (#F1804) was obtained from Millipore Sigma. GFP Antibody (#sc-9996) was purchased from Santa Cruz Biotechnology. H3 antibody was generated in-house. BRDT antibody was a gift from Dr. Lu Wang's lab at Northwestern University.

### GFP IP-MS

To prepare the GFP IP-MS samples, cells were released with trypsin and washed with PBS before incubation in cold Buffer A (10 mM HEPES pH 7.9, 10 mM KCl, 1.5 mM MgCl₂, DTT) (Thermo Scientific, #A39255), Protease inhibitor (Thermo Scientific, #PIA32963), and Phosphatase inhibitor (Thermo Scientific, #PIA32957)) for nuclear isolation. Nuclear pellets were lysed by the addition of cold Triton X-100 lysis buffer (50 mM Tris HCl, pH 8.0, 150 mM NaCl, 1.5 mM MgCl₂, 10% Glycerol, 0.5% Triton X-100, DTT, Protease inhibitor, and Phosphatase inhibitor) followed by rotation for 45 min and centrifugation @20,000 g for 15 minutes (all at 4° C.) The supernatant was incubated with ChromoTek GFP-Trap magnetic beads (Proteintech, #gtma) for >4 h at 4° C. for immunoprecipitation, then beads were washed 5× with cold Triton X-100 lysis buffer and 1× with cold Triton X-100 wash buffer (50 mM Tris HCl, pH 8.0, 150 mM NaCl, 1.5 mM MgCl₂, 1.5 mM EDTA, DTT). Glycine 2.5M pH 2.0 was used to elute the immunoprecipitated proteins and Tris buffer pH>10 was used to neutralize the eluate at RT. Neutralized eluate was snap frozen for MS or mixed with Laemmli sample buffer and boiled for 5 minutes for western blot. MS was carried out by the Proteomics Core Facility at the University of Arkansas for Medical Sciences.

### ChIP-Seq

DLD1 cells were crosslinked with 15 ml 1% PFA (ThermoFisher, #28908) in PBS on 15 cm plates for 10 minutes at RT and quenched by the addition of 2 ml 2.5M glycine followed by shaking for 5 minutes. Crosslinked cells were collected by scraping. Chromatin sonication was performed for 10 minutes using a Covaris E200 set to 10% duty factor, 200 cycles per burst, and 140 peak intensity power. For endogenous protein ChIP, 10-20% of mouse embryonic fibroblasts (MEF) chromatin (prepared the same way as in DLD1 cells) was added to each sample as a spike-in control. For overexpressed protein ChIP, such as GFP-ChIP, 20 ng spike-in chromatin (Active Motif, #53083) and 2 ug spike-in antibody (Active Motif, #61686) were added to each sample. Immunoprecipitation was carried out at 4° C. overnight using 5ul of Rpb1 (Cell Signaling Technology, #D8L4Y), 5ul of Ser2p (Active Motif, #61984), 10ul of CCNT1 (Cell Signaling Technology, #81464), 10ul of CDK9 (Santa Cruz Biotechnology, #sc-13130 X) antibodies, or 200ul of a cocktail consisting of 50ul of each GFP antibody (DSHB, #GFP-G1, #GFP-12A6, #GFP-12E6, #GFP-8H11). Immune complexes were enriched with Protein G-coupled Dynabeads (Invitrogen, #10004D) at 4° C. for >=4 h, and incubated with proteinase K (Roche, #3115828001) to reverse crosslink at 65° C. overnight. Eluted DNA was purified with the QIAquick PCR Purification Kit (Qiagen, #28106), and libraries were prepared using the KAPA HTP library preparation kit (Roche, #07961901001) for sequencing on the NovaSeq 6000.

### ChIP-Seq Analysis

Reads were aligned to hg38 with bowtie 1/250. Genes (N=6,481) with pausing site and TES annotation were obtained from a previously published study51. Promoter regions were designated as spanning from 100 bp upstream to 300 bp downstream of the pausing site. Gene body regions were designated as spanning from 300 bp downstream of the pausing site to the TES. FeatureCounts 2.0.152 was used to calculate the total mapped reads from Pol II ChIP-seq at promoters and within gene bodies. PRR is calculated as the ratio of Pol II signal density within the gene body to Pol II signal density at the promoter. BamCoverage in deeptools 3.1.153 was used to extend ChIP-seq reads to 150 bp. Log 2FC of ChIP-seq was calculated using bigwigCompare in deeptools with the following nondefault options: binSize 10, pseudocount 0.1. Heatmaps and metagene plots were also generated in deeptools. ECDFs and boxplots/scatterplots for the log 2PRR and MS were generated using R. Tracks were visualized in igv 2.13.2 (Broad Institute).

### REFERENCES

1. Core, L., and Adelman, K. (2019). Promoter-proximal pausing of RNA polymerase II: A nexus of gene regulation. Genes Dev 33, 960-982. 10.1101/gad.325142.119.
2. Chen, F. X., Smith, E. R., and Shilatifard, A. (2018). Born to run: Control of transcription elongation by RNA polymerase II. Nat Rev Mol Cell Biol 19, 464-478. 10.1038/s41580-018-0010-5.
3. Adelman, K., and Lis, J. T. (2012). Promoter-proximal pausing of RNA polymerase II: emerging roles in metazoans. Nat Rev Genet 13, 720-731. 10.1038/nrg3293.
4. Price, D. H. (2000). P-TEFb, a Cyclin-Dependent Kinase Controlling Elongation by RNA Polymerase II.
5. Peterlin, B. M., and Price, D. H. (2006). Controlling the Elongation Phase of Transcription with P-TEFb. Mol Cell 23, 297-305. 10.1016/j.molcel.2006.06.014.
6. Buratowski, S. (2009). Progression through the RNA Polymerase II CTD Cycle. Mol Cell 36, 541-546. 10.1016/j.molcel.2009.10.019.
7. Hsin, J. P., and Manley, J. L. (2012). The RNA polymerase II CTD coordinates transcription and RNA processing. Genes Dev 26, 2119-2137. 10.1101/gad.200303.112.
8. Lu, X., Zhu, X., Li, Y., Liu, M., Yu, B., Wang, Y., Rao, M., Yang, H., Zhou, K., Wang, Y., et al. (2016). Multiple P-TEFbs cooperatively regulate the release of promoter-proximally paused RNA polymerase II. Nucleic Acids Res 44, 6853-6867. 10.1093/nar/gkw571.
9. Lin, C., Smith, E. R., Takahashi, H., Lai, K. C., Martin-Brown, S., Florens, L., Washburn, M. P., Conaway, J. W., Conaway, R. C., and Shilatifard, A. (2010). AFF4, a Component of the ELL/P-TEFb Elongation Complex and a Shared Subunit of MLL Chimeras, Can Link Transcription Elongation to Leukemia. Mol Cell 37, 429-437. 10.1016/j.molcel.2010.01.026.
10. Li, Q., Price, J. P., Byers, S. A., Cheng, D., Peng, J., and Price, D. H. (2005). Analysis of the large inactive P-TEFb complex indicates that it contains one 7SK molecule, a dimer of HEXIM1 or HEXIM2, and two P-TEFb molecules containing Cdk9 phosphorylated at threonine 186. Journal of Biological Chemistry 280, 28819-28826. 10.1074/jbc.M502712200.
11. Smith, E., Lin, C., and Shilatifard, A. (2011). The super elongation complex (SEC) and MLL in development and disease. Genes Dev 25, 661-672. 10.1101/gad.2015411.
12. Zheng, B., Aoi, Y., Shah, A. P., Iwanaszko, M., Das, S., Rendleman, E. J., Zha, D., Khan, N., Smith, E. R., and Shilatifard, A. (2021). Acute perturbation strategies in interrogating RNA polymerase II elongation factor function in gene expression. Genes Dev 35, 273-285. 10.1101/GAD.346106.120.
13. Wu, S. Y., and Chiang, C. M. (2007). The double bromodomain-containing chromatin adaptor Brd4 and transcriptional regulation. Journal of Biological Chemistry 282, 13141-13145. 10.1074/jbc.R700001200.
14. Gaucher, J., Boussouar, F., Montellier, E., Curtet, S., Buchou, T., Bertrand, S., Hery, P., Jounier, S., Depaux, A., Vitte, A. L., et al. (2012). Bromodomain-dependent stage-specific male genome programming by Brdt. EMBO Journal 37, 3809-3820. 10.1038/emboj.2012.233.
15. Zhang, T., Cooper, S., and Brockdorff, N. (2015). The interplay of histone modifications-writers that read. EMBO Rep 16, 1467-1481. 10.15252/embr.201540945.
16. Atlasi, Y., and Stunnenberg, H. G. (2017). The interplay of epigenetic marks during stem cell differentiation and development. Nat Rev Genet 18, 643-658. 10.1038/nrg.2017.57.
17. Donati, B., Lorenzini, E., and Ciarrocchi, A. (2018). BRD4 and Cancer: Going beyond transcriptional regulation. Mol Cancer 17. 10.1186/s12943-018-0915-9.
18. Creyghton, M. P., Cheng, A. W., Welstead, G. G., Kooistra, T., Carey, B. W., Steine, E. J., Hanna, J., Lodato, M. A., Frampton, G. M., Sharp, P. A., et al. (2010). Histone H3K27ac separates active from poised enhancers and predicts developmental state. Proceedings of the National Academy of Sciences 107, 21931-21936. 10.1073/pnas. 1016071107.
19. Kanno, T., Kanno, Y., Leroy, G., Campos, E., Sun, H. W., Brooks, S. R., Vahedi, G., Heightman, T. D., Garcia, B. A., Reinberg, D., et al. (2014). BRD4 assists elongation of both coding and enhancer RNAs by interacting with acetylated histones. Nat Struct Mol Biol 27, 1047-1057. 10.1038/nsmb.2912.
20. Shorstova, T., Foulkes, W. D., and Witcher, M. (2021). Achieving clinical success with BET inhibitors as anti-cancer agents. Br J Cancer 124, 1478-1490. 10.1038/s41416-021-01321-0.
21. Winter, G. E., Mayer, A., Buckley, D. L., Erb, M. A., Roderick, J. E., Vittori, S., Reyes, J. M., di Julio, J., Souza, A., Ott, C. J., et al. (2017). BET Bromodomain Proteins Function as Master Transcription Elongation Factors Independent of CDK9 Recruitment. Mol Cell 67, 5-18.e19. 10.1016/j.molcel.2017.06.004.
22. Mertz, J. A., Conery, A. R., Bryant, B. M., Sandy, P., Balasubramanian, S., Mele, D. A., Bergeron, L., and Sims, R. J. (2011). Targeting MYC dependence in cancer by inhibiting BET bromodomains. Proc Natl Acad Sci USA 108, 16669-16674. 10.1073/pnas. 1108190108.
23. Lu, J., Qian, Y., Altieri, M., Dong, H., Wang, J., Raina, K., Hines, J., Winkler, J. D., Crew, A. P., Coleman, K., et al. (2015). Hijacking the E3 Ubiquitin Ligase Cereblon to Efficiently Target BRD4. Chem Biol 22, 755-763. 10.1016/j.chembiol.2015.05.009.
24. Wang, Z., Chivu, A. G., Choate, L. A., Rice, E. J., Miller, D. C., Chu, T., Chou, S. P., Kingsley, N. B., Petersen, J. L., Finno, C. J., et al. (2022). Prediction of histone post-translational modification patterns based on nascent transcription data. Nat Genet 54, 295-305. 10.1038/s41588-022-01026-x.
25. Winter, G. E., Buckley, D. L., Paulk, J., Roberts, J. M., Souza, A., Dhe-Paganon, S., and Bradner, J. E. (2015). Phthalimide conjugation as a strategy for in vivo target protein degradation. Science (1979) 348, 1376-1381. 10.1126/science.aab1433.
26. Sankar, A., Mohammad, F., Sundaramurthy, A. K., Wang, H., Lerdrup, M., Tatar, T., and Helin, K. (2022). Histone editing elucidates the functional roles of H3K27 methylation and acetylation in mammals. Nat Genet 54, 754-760. 10.1038/s41588-022-01091-2.
27. Zhang, T., Zhang, Z., Dong, Q., Xiong, J., and Zhu, B. (2020). Histone H3K27 acetylation is dispensable for enhancer activity in mouse embryonic stem cells. Genome Biol 21. 10.1186/s13059-020-01957-w.
28. Muhar, M., Ebert, A., Neumann, T., Umkehrer, C., Jude, J., Wieshofer, C., Rescheneder, P., Lipp, J. J., Herzog, V. A., Reichholf, B., et al. SLAM-seq defines direct gene-regulatory functions of the BRD4-MYC axis.
29. Arnold, M., Bressin, A., Jasnovidova, O., Meierhofer, D., and Mayer, A. (2021). A BRD4-mediated elongation control point primes transcribing RNA polymerase II for 3′-processing and termination. Mol Cell 81, 3589-3603.e13. 10.1016/j.molcel.2021.06.026.
30. Li, S., Prasanna, X., Salo, V. T., Vattulainen, I., and Ikonen, E. (2019). An efficient auxininducible degron system with low basal degradation in human cells. Nat Methods 16, 866-869. 10.1038/s41592-019-0512-x.
31. Lovén, J., Hoke, H. A., Lin, C. Y., Lau, A., Orlando, D. A., Vakoc, C. R., Bradner, J. E., Lee, T. I., and Young, R. A. (2013). Selective inhibition of tumor oncogenes by disruption of superenhancers. Cell 153, 320-334. 10.1016/j.cell.2013.03.036.
32. Itzen, F., Greifenberg, A. K., Bösken, C. A., and Geyer, M. (2014). Brd4 activates P-TEFb for RNA polymerase II CTD phosphorylation. Nucleic Acids Res 42, 7577-7590. 10.1093/nar/gku449.
33. Moon, K. J., Mochizuki, K., Zhou, M., Jeong, H. S., Brady, J. N., and Ozato, K. (2005). The bromodomain protein Brd4 is a positive regulatory component of P-TEFb and stimulates RNA polymerase II-dependent transcription. Mol Cell 19, 523-534. 10.1016/j.molcel.2005.06.027.
34. Phatnani, H. P., and Greenleaf, A. L. (2006). Phosphorylation and functions of the RNA polymerase II CTD. Genes Dev 20, 2922-2936. 10.1101/gad.1477006.
35. Moon, K. J., Mochizuki, K., Zhou, M., Jeong, H. S., Brady, J. N., and Ozato, K. (2005). The bromodomain protein Brd4 is a positive regulatory component of P-TEFb and stimulates RNA polymerase II-dependent transcription. Mol Cell 19, 523-534. 10.1016/j.molcel.2005.06.027.
36. Yang, Z., He, N., and Zhou, Q. (2008). Brd4 Recruits P-TEFb to Chromosomes at Late Mitosis To Promote G 1 Gene Expression and Cell Cycle Progression. Mol Cell Biol 28, 967-976. 10.1128/mcb.01020-07.
37. Tahirov, T. H., Babayeva, N. D., Varzavand, K., Cooper, J. J., Sedore, S. C., and Price, D. H. (2010). Crystal structure of HIV-1 Tat complexed with human P-TEFb. Nature 465, 747-751. 10.1038/nature09131.
38. Pettersen, E. F., Goddard, T. D., Huang, C. C., Meng, E. C., Couch, G. S., Croll, T. I., Morris, J. H., and Ferrin, T. E. (2021). UCSF ChimeraX: Structure visualization for researchers, educators, and developers. Protein Science 30, 70-82. 10.1002/pro.3943.
39. Shu, S., Lin, C. Y., He, H. H., Witwicki, R. M., Tabassum, D. P., Roberts, J. M., Janiszewska, M., Huh, S. J., Liang, Y., Ryan, J., et al. (2016). Response and resistance to BET bromodomain inhibitors in triple-negative breast cancer. Nature 529, 413-417. 10.1038/nature16508.
40. Fong, C. Y., Gilan, O., Lam, E. Y. N., Rubin, A. F., Ftouni, S., Tyler, D., Stanley, K., Sinha, D., Ych, P., Morison, J., et al. (2015). BET inhibitor resistance emerges from leukaemia stem cells. Nature 525, 538-542. 10.1038/nature14888.
41. Rathert, P., Roth, M., Neumann, T., Muerdter, F., Roc, J. S., Muhar, M., Deswal, S., Cerny-Reiterer, S., Peter, B., Jude, J., et al. (2015). Transcriptional plasticity promotes primary and acquired resistance to BET inhibition. Nature 525, 543-547. 10.1038/nature14898.
42. Calder, J., Nagelberg, A., Luu, J., Lu, D., and Lockwood, W. W. (2021). Resistance to BET inhibitors in lung adenocarcinoma is mediated by casein kinase phosphorylation of BRD4. Oncogenesis 10. 10.1038/s41389-021-00316-z.
43. Sun, Y., Zhang, Z., Zhang, K., Liu, Y., Shen, P., Cai, M., Jia, C., Wang, W., Gu, Z., Ma, P., et al. (2021). Epigenetic heterogeneity promotes acquired resistance to BET bromodomain inhibition in ovarian cancer.
44. Shu, S., Lin, C. Y., He, H. H., Witwicki, R. M., Tabassum, D. P., Roberts, J. M., Janiszewska, M., Huh, S. J., Liang, Y., Ryan, J., et al. (2016). Response and resistance to BET bromodomain inhibitors in triple-negative breast cancer. Nature 529, 413-417. 10.1038/nature16508.
45. Andrieu, G., Belkina, A. C., and Denis, G. v. (2016). Clinical trials for BET inhibitors run ahead of the science. Drug Discov Today Technol 19, 45-50. 10.1016/j.ddtec.2016.06.004.
46. Mahat, D. B., Salamanca, H. H., Duarte, F. M., Danko, C. G., and Lis, J. T. (2016). Mammalian Heat Shock Response and Mechanisms Underlying Its Genome-wide Transcriptional Regulation. Mol Cell 62, 63-78. 10.1016/j.molcel.2016.02.025.
47. Cheung, K. L., Zhang, F., Jaganathan, A., Sharma, R., Zhang, Q., Konuma, T., Shen, T., Lee, J. Y., Ren, C., Chen, C. H., et al. (2017). Distinct Roles of Brd2 and Brd4 in Potentiating the Transcriptional Program for Th17 Cell Differentiation. Mol Cell 65, 1068-1080.e5. 10.1016/j.molcel.2016.12.022.
48. Wang, C., Xu, Q., Zhang, X., Day, D. S., Abraham, B. J., Lun, K., Chen, L., Huang, J., and Ji, X. (2022). BRD2 interconnects with BRD3 to facilitate Pol II transcription initiation and elongation to prime promoters for cell differentiation. Cellular and Molecular Life Sciences 79. 10.1007/s00018-022-04349-4.
49. Hsu, S. C., Gilgenast, T. G., Bartman, C. R., Edwards, C. R., Stonestrom, A. J., Huang, P., Emerson, D. J., Evans, P., Werner, M. T., Keller, C. A., et al. (2017). The BET Protein BRD2 Cooperates with CTCF to Enforce Transcriptional and Architectural Boundaries. Mol Cell 66, 102-116.e7. 10.1016/j.molcel.2017.02.027.
50. Langmead, B., and Salzberg, S. L. (2012). Fast gapped-read alignment with Bowtie 2. Nat Methods 9, 357-359. 10.1038/nmeth. 1923.
51. Aoi, Y., Shah, A. P., Ganesan, S., Soliman, S. H. A., Cho, B. K., Goo, Y. A., Kelleher, N. L., and Shilatifard, A. (2022). SPT6 functions in transcriptional pause/release via PAFIC recruitment. Mol Cell 82, 3412-3423.e5. 10.1016/j.molcel.2022.06.037.
52. Liao, Y., Smyth, G. K., and Shi, W. (2014). FeatureCounts: An efficient general purpose program for assigning sequence reads to genomic features. Bioinformatics 30, 923-930. 10.1093/bioinformatics/btt656.
53. Ramírez, F., Ryan, D. P., Grüning, B., Bhardwaj, V., Kilpert, F., Richter, A. S., Heyne, S., Dündar, F., and Manke, T. (2016). deepTools2: a next generation web server for deep-sequencing data analysis. Nucleic Acids Res 44, W160-W165. 10.1093/NAR/GKW257.

### Example 2

### Summary

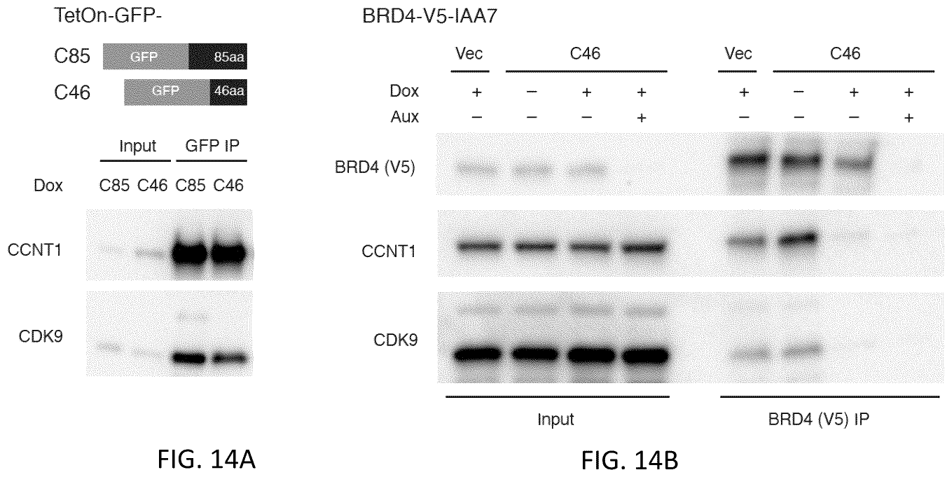

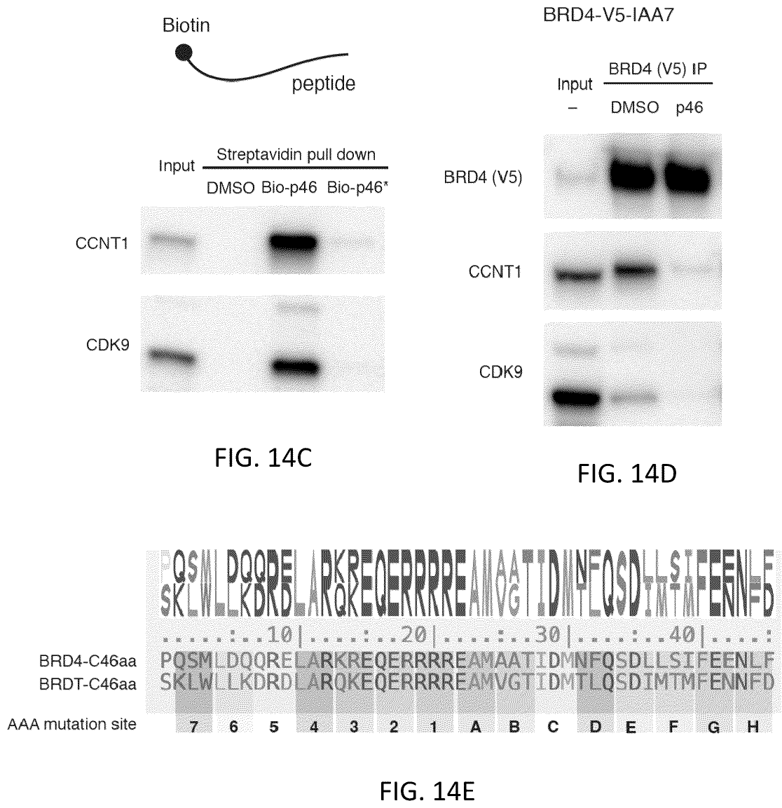

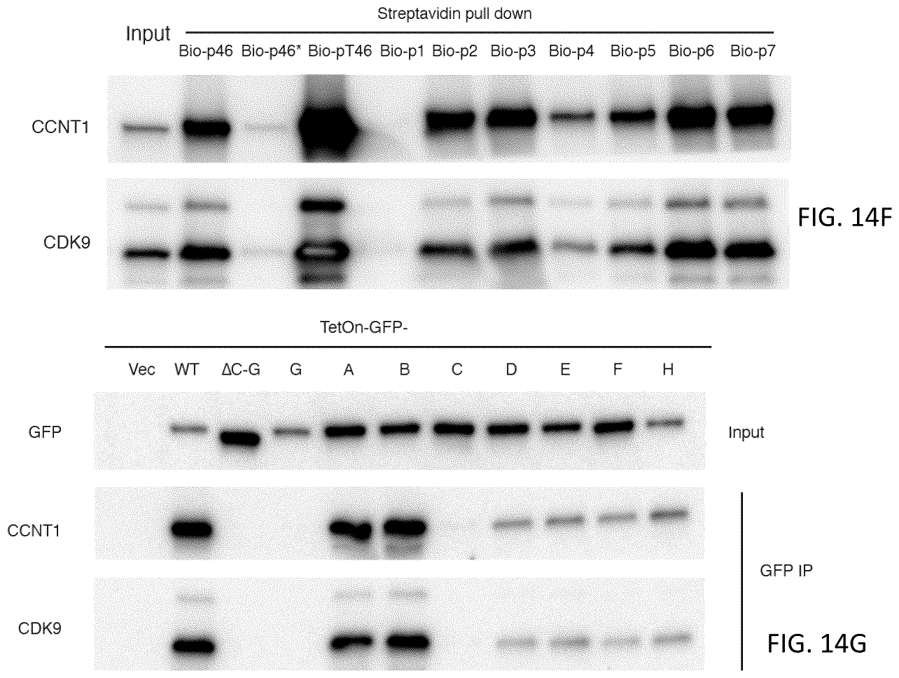

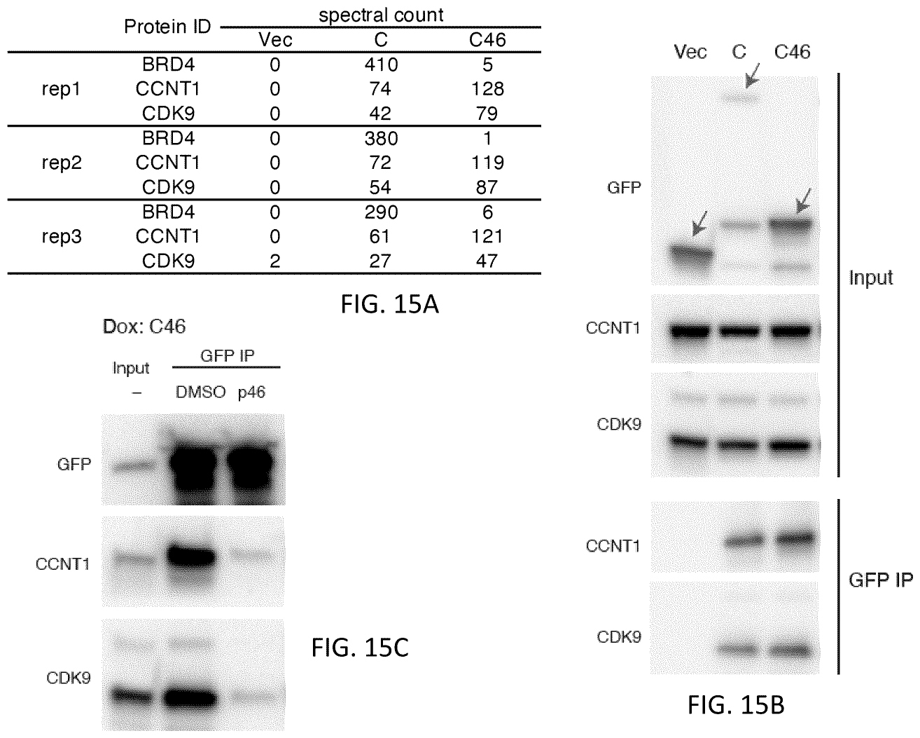

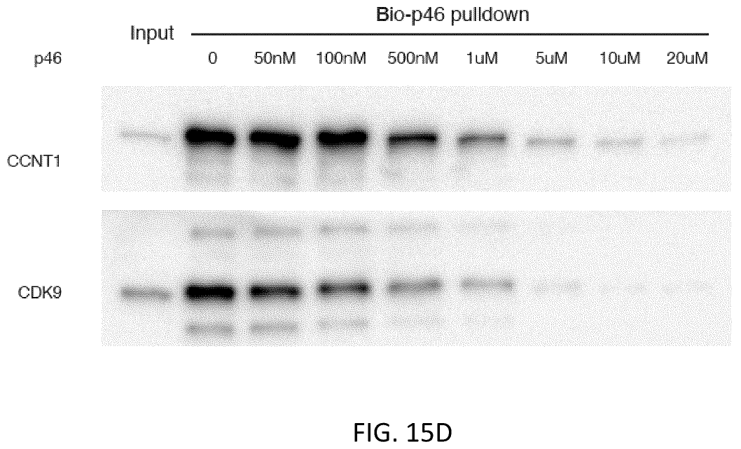

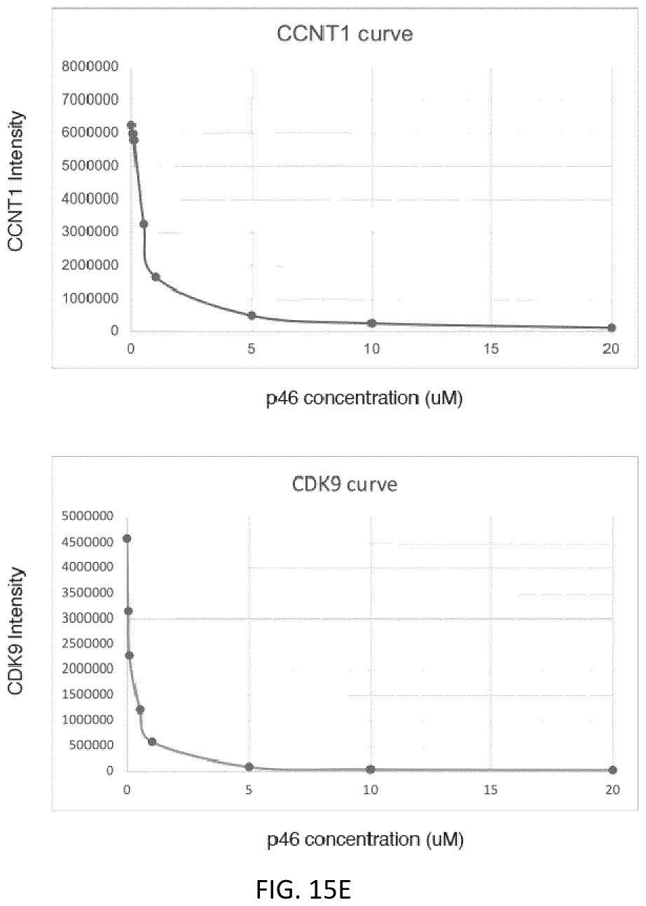

Knowing the exact PTEFb binding site(s) in BRD4 C-terminus will help guide the screening for small molecules targeting BRD4 C-terminus. To determine the minimum pTEFb binding domain, we chopped the BRD4 C terminus further into 85aa and 46aa and expressed in cells with a GFP tag (C85 and C46). Western blot for GFP IP of these mutants showed that both C85 and C46 bind to PTEFb strongly (FIG. 14A). Compared to the C terminus 340aa (C), length reduction to 46aa (PQSMLDQQRELARKREQERRRREAMAATIDMNFQSDLLSIFEENLF (SEQ ID NO: 1)) does not impair its interaction with PTEFb, as indicated by the GFP IP-MS and validated by western blot (FIGS. 15A and 15B). When C46 is expressed in cells, it competes with the endogenous BRD4 for PTEFb (FIG. 14B). Synthetic peptide of the biotin-labelled 46aa (Bio-p46) is able to pull down PTEFb from cell lysate, while the known loss of function mutation of FEE residues at the C terminus tail to AAA1 (Bio-p46*) leads to dramatic decrease of PTEFb binding (FIG. 14C). Consequently, in the presence of synthetic peptide of the 46aa (p46), the amount of PTEFb bound to endogenous BRD4 or overexpressed GFP-tagged C46 is significantly decreased (FIGS. 14D and 15C), suggesting that synthetic peptide of the 46aa is structurally similar to the one that is expressed by cells. We then utilized the competition between free and biotin-labelled peptides to assess the “IC50” for the potential BRD4 C terminus inhibitor in vitro and determined it could be at hundred nanomole level (FIGS. 15D and 15E). We used both synthetic peptides and expressed GPF-tagged mutants to carry out the alanine scanning for all the 46 residues and found that in addition to the known FEE site,¹ there are also two other sites of RRR and IDM that are essential for PTEFb binding and conserved between BRD4 and BRDT (FIGS. 14E-14G).

### Methods

To pull down PTEFb by Biotin-labeled peptide, cells were lysed with Triton X-100 lysis buffer as used for GFP IP. Biotin-labeled peptides were pre-incubated with streptavidin beads at room temperature for 30 min. Conjugated beads were then washed with lysis buffer twice before adding to the cell lysate. Pulldown was carried out at 4° C. by rotating for >=4 h, followed by 5 times wash with lysis buffer. To elute, 4× Laemmli sample buffer was added and boiled for 10 min. For the peptide titration experiment, a series of concentration of p46 was pre-incubated with same amount of cell lysate for 1 h at 4° C. Same amount of Bio-p46 conjugated streptavidin beads were used to pull down PTEFb for >=4 h. PTEFb signal intensity from western blot was determined by Image Lab from Bio-Rad.

### REFERENCE

1. Bisgrove, D. A., Mahmoudi, T., Henklein, P., and Verdin, E. (2007). Conserved P-TEFb-interacting domain of BRD4 inhibits HIV transcription.

Sequences

C-terminal fragment C46-SEQ ID NO: 1-
PQSMLDQQRELARKREQERRRREAMAATIDMNFQSDLLSIFEENLF
C-terminal fragment Cs-SEQ ID NO: 2-
DLKIKNMGSWASLVQKHPTTPSSTAKSSSDSFEQFRRAAREKEEREKALKAQAEHAEKE
KERLRQERMRSREDEDALEQARRAHEEARRRQEQQQQQRQEQQQQQQQQAAAVAAA
ATPQAQSSQPQSMLDQQRELARKREQERRRREAMAATIDMNFQSDLLSIFEENLF
Full length BRD4-SEQ ID NO: 3
MSAESGPGTRLRNLPVMGDGLETSQMSTTQAQAQPQPANAASTNPPPPETSNPNKPKRQ
TNQLQYLLRVVLKTLWKHQFAWPFQQPVDAVKLNLPDYYKIIKTPMDMGTIKKRLENN
YYWNAQECIQDFNTMFTNCYIYNKPGDDIVLMAEALEKLFLQKINELPTEETEIMIVQAK
GRGRGRKETGTAKPGVSTVPNTTQASTPPQTQTPQPNPPPVQATPHPFPAVTPDLIVQTP
VMTVVPPQPLQTPPPVPPQPQPPPAPAPQPVQSHPPIIAATPQPVKTKKGVKRKADTTTPT
TIDPIHEPPSLPPEPKTTKLGQRRESSRPVKPPKKDVPDSQQHPAPEKSSKVSEQLKCCSGI
LKEMFAKKHAAYAWPFYKPVDVEALGLHDYCDIIKHPMDMSTIKSKLEAREYRDAQEF
GADVRLMFSNCYKYNPPDHEVVAMARKLQDVFEMRFAKMPDEPEEPVVAVSSPAVPP
PTKVVAPPSSSDSSSDSSSDSDSSTDDSEEERAQRLAELQEQLKAVHEQLAALSQPQQNK
PKKKEKDKKEKKKEKHKRKEEVEENKKSKAKEPPPKKTKKNNSSNSNVSKKEPAPMKS
KPPPTYESEEEDKCKPMSYEEKRQLSLDINKLPGEKLGRVVHIIQSREPSLKNSNPDEIEID
FETLKPSTLRELERYVTSCLRKKRKPQAEKVDVIAGSSKMKGFSSSESESSSESSSSDSED
SETEMAPKSKKKGHPGREQKKHHHHHHQQMQQAPAPVPQQPPPPPQQPPPPPPPQQQQ
QPPPPPPPPSMPQQAAPAMKSSPPPFIATQVPVLEPQLPGSVFDPIGHFTQPILHLPQPELPP
HLPQPPEHSTPPHLNQHAVVSPPALHNALPQQPSRPSNRAAALPPKPARPPAVSPALTQT
PLLPQPPMAQPPQVLLEDEEPPAPPLTSMQMQLYLQQLQKVQPPTPLLPSVKVQSQPPPP
LPPPPHPSVQQQLQQQPPPPPPPQPQPPPQQQHQPPPRPVHLQPMQFSTHIQQPPPPQGQQ
PPHPPPGQQPPPPQPAKPQQVIQHHHSPRHHKSDPYSTGHLREAPSPLMIHSPQMSQFQSL
THQSPPQQNVQPKKQELRAASVVQPQPLVVVKEEKIHSPIIRSEPFSPSLRPEPPKHPESIK
APVHLPQRPEMKPVDVGRPVIRPPEQNAPPPGAPDKDKQKQEPKTPVAPKKDLKIKNM
GSWASLVQKHPTTPSSTAKSSSDSFEQFRRAAREKEEREKALKAQAEHAEKEKERLRQE
RMRSREDEDALEQARRAHEEARRRQEQQQQQRQEQQQQQQQQAAAVAAAATPQAQS
SQPQSMLDQQRELARKREQERRRREAMAATIDMNFQSDLLSIFEENLF
BRD4 with C46 deletion-SEQ ID NO: 4
MSAESGPGTRLRNLPVMGDGLETSQMSTTQAQAQPQPANAASTNPPPPETSNPNKPKRQ
TNQLQYLLRVVLKTLWKHQFAWPFQQPVDAVKLNLPDYYKIIKTPMDMGTIKKRLENN
YYWNAQECIQDFNTMFTNCYIYNKPGDDIVLMAEALEKLFLQKINELPTEETEIMIVQAK
GRGRGRKETGTAKPGVSTVPNTTQASTPPQTQTPQPNPPPVQATPHPFPAVTPDLIVQTP
VMTVVPPQPLQTPPPVPPQPQPPPAPAPQPVQSHPPIIAATPQPVKTKKGVKRKADTTTPT
TIDPIHEPPSLPPEPKTTKLGQRRESSRPVKPPKKDVPDSQQHPAPEKSSKVSEQLKCCSGI
LKEMFAKKHAAYAWPFYKPVDVEALGLHDYCDIIKHPMDMSTIKSKLEAREYRDAQEF
GADVRLMFSNCYKYNPPDHEVVAMARKLQDVFEMRFAKMPDEPEEPVVAVSSPAVPP
PTKVVAPPSSSDSSSDSSSDSDSSTDDSEEERAQRLAELQEQLKAVHEQLAALSQPQQNK
PKKKEKDKKEKKKEKHKRKEEVEENKKSKAKEPPPKKTKKNNSSNSNVSKKEPAPMKS
KPPPTYESEEEDKCKPMSYEEKRQLSLDINKLPGEKLGRVVHIIQSREPSLKNSNPDEIEID
FETLKPSTLRELERYVTSCLRKKRKPQAEKVDVIAGSSKMKGFSSSESESSSESSSSDSED
SETEMAPKSKKKGHPGREQKKHHHHHHQQMQQAPAPVPQQPPPPPQQPPPPPPPQQQQ
QPPPPPPPPSMPQQAAPAMKSSPPPFIATQVPVLEPQLPGSVFDPIGHFTQPILHLPQPELPP
HLPQPPEHSTPPHLNQHAVVSPPALHNALPQQPSRPSNRAAALPPKPARPPAVSPALTQT
PLLPQPPMAQPPQVLLEDEEPPAPPLTSMQMQLYLQQLQKVQPPTPLLPSVKVQSQPPPP
LPPPPHPSVQQQLQQQPPPPPPPQPQPPPQQQHQPPPRPVHLQPMQFSTHIQQPPPPQGQQ
PPHPPPGQQPPPPQPAKPQQVIQHHHSPRHHKSDPYSTGHLREAPSPLMIHSPQMSQFQSL
THQSPPQQNVQPKKQELRAASVVQPQPLVVVKEEKIHSPIIRSEPFSPSLRPEPPKHPESIK
APVHLPQRPEMKPVDVGRPVIRPPEQNAPPPGAPDKDKQKQEPKTPVAPKKDLKIKNM
GSWASLVQKHPTTPSSTAKSSSDSFEQFRRAAREKEEREKALKAQAEHAEKEKERLRQE
RMRSREDEDALEQARRAHEEARRRQEQQQQQRQEQQQQQQQQAAAVAAAATPQAQSSQ
BRD4 with Cs deletion-SEQ ID NO: 5
MSAESGPGTRLRNLPVMGDGLETSQMSTTQAQAQPQPANAASTNPPPPETSNPNKPKRQ
TNQLQYLLRVVLKTLWKHQFAWPFQQPVDAVKLNLPDYYKIIKTPMDMGTIKKRLENN
YYWNAQECIQDFNTMFTNCYIYNKPGDDIVLMAEALEKLFLQKINELPTEETEIMIVQAK
GRGRGRKETGTAKPGVSTVPNTTQASTPPQTQTPQPNPPPVQATPHPFPAVTPDLIVQTP
VMTVVPPQPLQTPPPVPPQPQPPPAPAPQPVQSHPPIIAATPQPVKTKKGVKRKADTTTPT
TIDPIHEPPSLPPEPKTTKLGQRRESSRPVKPPKKDVPDSQQHPAPEKSSKVSEQLKCCSGI
LKEMFAKKHAAYAWPFYKPVDVEALGLHDYCDIIKHPMDMSTIKSKLEAREYRDAQEF
GADVRLMFSNCYKYNPPDHEVVAMARKLQDVFEMRFAKMPDEPEEPVVAVSSPAVPP
PTKVVAPPSSSDSSSDSSSDSDSSTDDSEEERAQRLAELQEQLKAVHEQLAALSQPQQNK
PKKKEKDKKEKKKEKHKRKEEVEENKKSKAKEPPPKKTKKNNSSNSNVSKKEPAPMKS
KPPPTYESEEEDKCKPMSYEEKRQLSLDINKLPGEKLGRVVHIIQSREPSLKNSNPDEIEID
FETLKPSTLRELERYVTSCLRKKRKPQAEKVDVIAGSSKMKGFSSSESESSSESSSSDSED
SETEMAPKSKKKGHPGREQKKHHHHHHQQMQQAPAPVPQQPPPPPQQPPPPPPPQQQQ
QPPPPPPPPSMPQQAAPAMKSSPPPFIATQVPVLEPQLPGSVFDPIGHFTQPILHLPQPELPP
HLPQPPEHSTPPHLNQHAVVSPPALHNALPQQPSRPSNRAAALPPKPARPPAVSPALTQT
PLLPQPPMAQPPQVLLEDEEPPAPPLTSMQMQLYLQQLQKVQPPTPLLPSVKVQSQPPPP
LPPPPHPSVQQQLQQQPPPPPPPQPQPPPQQQHQPPPRPVHLQPMQFSTHIQQPPPPQGQQ
PPHPPPGQQPPPPQPAKPQQVIQHHHSPRHHKSDPYSTGHLREAPSPLMIHSPQMSQFQSL
THQSPPQQNVQPKKQELRAASVVQPQPLVVVKEEKIHSPIIRSEPFSPSLRPEPPKHPESIK
APVHLPQRPEMKPVDVGRPVIRPPEQNAPPPGAPDKDKQKQEPKTPVAPKK
AATCTTTTCTGAGCGCACCT-SEQ ID NO.: 6
ATCAAAGTCAGAAGCCACCT-SEQ ID NO.: 7
SKLWLLKDRDLARQKEQERRRREAMVGTIDMTLQSDIMTMFENNFD-SEQ ID NO: 8

1. An engineered bromodomain-containing protein 4 (BRD4) polypeptide comprising a deletion of at least one of bromodomain 1 (BD1), bromodomain 2 (BD2), and extra terminal domain (ET).

2. The engineered BRD4 polypeptide of claim 1 comprising a deletion of each of BD1, BD2, and ET.

3. The engineered BRD4 polypeptide of claim 1, wherein the polypeptide comprises a C-terminal fragment of SEQ ID NO: 1 or SEQ ID NO: 2.

4. The engineered BRD4 polypeptide of claim 3, wherein the polypeptide consists of the C terminal fragment of SEQ ID NO: 1 or SEQ ID NO: 2.

5. A polynucleotide encoding the engineered BRD4 polypeptide of claim 1.

6. A construct comprising the polynucleotide of claim 5 operably linked to a promoter.

7. The construct of claim 6, wherein the promoter is an inducible promoter.

8. The construct of claim 6, further comprising a sequence encoding a protein tag.

9. The construct of claim 8, wherein the tag is a Flag tag or a GFP tag.

10. A vector comprising the polynucleotide of claim 5.

11. A cell comprising the engineered polypeptide of claim 1.

12. The cell of claim 11, wherein the cell is depleted of endogenous BRD4.

13. An engineered bromodomain-containing protein 4 (BRD4) polypeptide comprising a deletion of a C-terminal fragment of SEQ ID NO: 1 or SEQ ID NO: 2.

14. A cell comprising the engineered polypeptide of claim 13.

15. A kit comprising the engineered BRD4 polypeptide of claim 1, and further comprising a second engineered BRD4 polypeptide comprising a deletion of a C terminal fragment of SEQ ID NO: 1 or SEQ ID NO: 2.

16. A method for identifying a candidate agent as a BRD4 C-terminal specific inhibitor, the method comprising:
(a) measuring Pol II pausing in a first population of cells, wherein the first population of cells comprises the cell of claim 12;
(b) contacting the first population of cells with the candidate agent; and
(c) measuring Pol II pausing in the contacted first population of cells, wherein an increase in measured Pol II pausing in step (c) compared to measured Pol II pausing in step (a) identifies the candidate agent as a BRD4 C-terminal specific inhibitor.

17. The method of claim 16, further comprising:
(d) measuring Pol II pausing in a second population of cells, wherein the second population of cells comprises a cell comprising an engineered bromodomain-containing protein 4 (BRD4) polypeptide comprising a deletion of a C-terminal fragment of SEQ ID NO: 1 or SEQ ID NO: 2, wherein the cell is depleted of endogenous BRD4;
(e) contacting the second population of cells with the candidate BRD4 C-terminal specific inhibitor; and
(f) measuring Pol II pausing in the contacted second population of cells, wherein similar or reduced measured Pol II pausing in step (f) compared to measured Pol II pausing in step (d) further identifies the candidate agent as a BRD4 C-terminal specific inhibitor.

18. The method of claim 16, wherein Pol II pausing is measured by at least one of a cell growth assay and a Pol II-DNA binding assay.

19. The method of claim 17, wherein the first population of cells and the second population of cells are DLD1 cells or NCIH2009 cells.

20. A method for increasing RNA polymerase II (Pol II) pause release in a cell, the method comprising introducing the engineered BRD4 polypeptide of claim 1 into the cell.
